(19) United States
(12) Patent Application Publication
(10) Pub. No.: US 2025/0345407 A1
(43) Pub. Date: Nov. 13, 2025

(54) NUCLEIC ACID BASED VACCINE ENCODING AN ESCHERICHIA COLI FIMH ANTIGENIC POLYPEPTIDE

(71) Applicant: CureVac SE, Tübingen (DE)

(72) Inventors: Roberto ADAMO, Siena (IT); Hans Wolfgang GROSSE, Tübingen (DE); Benjamin PETSCH, Tübingen (DE); Sanjay PHOGAT, Siena (IT); Susanne RAUCH, Tübingen (DE); Sandro ROIER, Tübingen (DE); Roberto ROSINI, Siena (IT)

(73) Assignee: CureVac SE, Tübingen (DE)

(21) Appl. No.: 18/868,888

(22) PCT Filed: May 23, 2023
(86) PCT No.: PCT/EP2023/063799

§ 371 Date: Nov. 25, 2024

Related U.S. Application Data
(60) Provisional application No. 63/345,610, filed on May 25, 2022.

(51) Int. Cl. A61K 39/108; A61K 39/00; A61P 37/04; C07K 14/245

(52) U.S. Cl. CPC A61K 39/0258; A61P 37/04; C07K 14/245; A61K 2039/53; A61K 2039/6018

(57) ABSTRACT

The disclosure is directed to a coding RNA encoding an antigenic polypeptide which is selected or derived from Escherichia coli FimH. The present disclosure is also directed to compositions and vaccines comprising said coding RNA. Further, the disclosure concerns a kit, particularly a kit of parts comprising the coding RNA, or the composition, or the vaccine. The disclosure is also directed to methods of treating or preventing a disorder caused by E. coli.

### SEQUENCE LISTING

The instant application contains an electronically submitted ST.26 Sequence Listing in XML file format (production date 2023-05-19) which is hereby incorporated by reference in its entirety. Additional sequences shorter than 10 specifically defined nucleotides or 4 specifically defined amino acid are disclosed in Table 13.

### FIELD OF THE INVENTION

The present disclosure is directed to a coding RNA encoding an antigenic polypeptide which is selected or derived from Escherichia coli FimH. The disclosure is also directed to pharmaceutical compositions, vaccines, kits or kits of parts suitable for use in the treatment and/or prevention of disease, in particular, urinary tract infection (UTI).

### BACKGROUND TO THE INVENTION

Uropathogenic Escherichia coli (UPEC), a subgroup of Extraintestinal Pathogenic Escherichia coli (ExPEC), causes the vast majority of urinary tract infections (UTIs) and is a leading cause of adult bacteraemia as well as the second most common cause of neonatal meningitis. UTIs are commonly treated with antibiotics but the emergence of multi-drug resistant pathogens has highlighted the need for an effective vaccine to prevent both uncomplicated and complicated urinary tract infections (Flores-Mireles A L, et als. Nat Rev Microbiol. 2015 May; 13(5):269-84). The tip-localized adhesin FimH of the type 1 pili (type 1 fimbriae D-mannose specific adhesin) allows ExPEC to colonize the bladder epithelium during UTIs by binding to mannosylated receptors on the urothelial surface (Mulvey M A, et al. Science. 1998 Nov. 20; 282(5393):1494-7). Full-length FimH is composed of two domains connected by a 5-amino acid linker: an N-terminal lectin domain (FimHL), which binds mannose on urothelial cells receptors, and a C-terminal pilin domain (FimHP). The pilin domain (FimHP) has an (Ig)-like fold but lacks the seventh C-terminal beta strand. The absence of a strand produces a deep groove along the surface of FimHP and exposes its hydrophobic core, thereby accounting for the instability of FimH when expressed without a chaperone. In the chaperone:subunit complex FimHP interacts non-covalently with a donor strand either of the chaperone FimC in the periplasm, or of the subsequent subunit of the assembled pilus (FimG) in a process known as donor strand complementation or donor strand exchange, respectively, which simultaneously stabilizes the pilus subunits and caps their interactive surfaces.

The lectin domain (FimHL) is known to adopt two conformations with different mannose-binding affinity—a high-affinity conformation, also known as relaxed (R) state, and a low-affinity conformation, also known as tense (T) state. The in vivo conformation of FimH is influenced by flow conditions, shear stress conditions being known to induce a high-mannose binding conformation, and is also at least partly determined by the in vivo interaction with the FimH binding proteins FimG or FimC.

Antibodies binding to FimH prevent colonization and facilitate the clearance of the bacteria by inhibiting bacterial adhesion to the urinary tract (Langermann S, et al. Science. 1997 Apr. 25; 276(5312):607-11). Particularly, monoclonal antibodies against FimHL in the low affinity conformation have been shown to provide an improved inhibition of adhesion to the bladder (Tchesnokova et al. Infect Immun. 2011 October; 79(10):3895-904). Transudation of serum IgGs in the urogenital tract seems responsible for inhibiting bacterial adhesion.

Therefore, FimH is considered as a promising vaccine antigen. However, manufacturing FimH at commercial scale is challenging, as FimH needs to be produced in sufficient amounts and in the conformation capable of eliciting functional antibodies.

Clinical trials testing a four-dose regimen of FimH complexed with its chaperon FimC (FimHC) and formulated with the adjuvant PHAD have been reported (Eldridge G R, et al. Hum Vaccin Immunother. 2021 May 4; 17(5):1262-1270). While FimC seems to prevent FimH degradation, providing FimHC complexes involves significant production burdens.

Alternative strategies for recombinant production of FimH in a functional conformation have also been reported, such as engineering of the mannose pocket (Kisiela D I, et al. Proc Natl Acad Sci USA. 2013 Nov. 19; 110(47):19089-94), complexing FimH with a recombinant donor strand peptide of FimG (Sauer M M, et al. Nat Commun. 2016 Mar. 7; 7:10738), or mammalian cell expression of a FimH stabilized by a donor strand peptide of FimG.

Therefore, there remains a need to overcome the challenges due to recombinant production of FimH-based vaccines and to provide immunogenic compositions capable of eliciting a rapid and robust immune response against ExPEC.

### SUMMARY OF THE INVENTION

In a first aspect of the invention it is provided a coding RNA comprising at least one untranslated region (UTR); and at least one coding sequence encoding an antigenic polypeptide which is selected or derived from Escherichia coli (“E. coli”, “Ec”) type 1 fimbriae D-mannose specific adhesin (FimH). In one embodiment the E. coli FimH comprises an amino acid sequence which is identical or at least 70%, 80%, 85%, 86%, 87%, 88%, 89%, 90%, 91%, 92%, 93%, 94%, 95%, 96%, 97%, 98%, or 99% identical to any one of SEQ ID NOs: 177-186, 247-256 or is an immunogenic fragment or immunogenic variant thereof.

In some embodiments, the coding sequence additionally encodes one or more further peptide or protein elements selected from: a donor strand peptide, a signal peptide, an antigen clustering domain, or a transmembrane domain. In one embodiment, the further peptide or protein element is a donor strand peptide, and optionally the coding sequence encodes the following elements in N-terminal to C-terminal direction: the antigenic polypeptide which is selected or derived from Escherichia coli FimH; and the donor strand peptide.

In one embodiment, the donor strand peptide comprises or consists of the amino acid sequence of SEQ ID NO: 338 or a variant thereof, optionally wherein the variant of SEQ ID NO: 338 has from 1 to 5, such as 1, 2, 3 or 4 single amino acid mutations compared to SEQ ID NO: 338. In one embodiment, the coding sequence additionally encodes a peptide linker element, and optionally the coding sequence encodes the following elements in N-terminal to C-terminal direction: the antigenic polypeptide which is selected or derived from Escherichia coli FimH; the peptide linker element; and the donor strand peptide. In one embodiment, the peptide linker element comprises or consists of SEQ ID NO: 352.

In one embodiment, the antigenic peptide is in a low mannose binding affinity conformation. In certain embodiments, the coding sequence additionally encodes an antigen clustering domain, and optionally the antigen clustering domain is selected or derived from ferritin or lumazine synthase. In some additional embodiments, the amino acid sequence of the antigen clustering domain is identical or at least 70%, 80%, 85%, 86%, 87%, 88%, 89%, 90%, 91%, 92%, 93%, 94%, 95%, 96%, 97%, 98%, or 99% identical to any one of amino acid sequences SEQ ID NOs: 457-459, 443, 444, or fragment or variant thereof.

In certain embodiments, the coding sequence additionally encodes a signal peptide, and optionally the signal peptide is or is derived from immunoglobulin E (IgE) or immunoglobulin Kappa (IgK). In some additional embodiments, the amino acid sequences of said signal peptides is identical or at least 70%, 80%, 85%, 86%, 87%, 88%, 89%, 90%, 91%, 92%, 93%, 94%, 95%, 96%, 97%, 98%, or 99% identical to any one of amino acid sequences SEQ ID NOs: 394, 395, or fragment or variant thereof.

In some embodiments, the coding sequence encodes the following elements optionally in N-terminal to C-terminal direction: (a) signal peptide, antigenic polypeptide; (b) signal peptide, antigenic polypeptide, peptide linker, donor strand peptide; (c) antigen clustering domain, peptide linker, antigenic polypeptide, peptide linker, donor strand peptide; (d) signal peptide, antigen clustering domain, peptide linker, antigenic polypeptide, peptide linker, donor strand peptide; (e) signal peptide, antigenic polypeptide, peptide linker, donor strand peptide, peptide linker, antigen clustering domain; or (f) signal peptide, antigenic polypeptide, peptide linker, donor strand peptide, peptide linker, transmembrane domain.

In some embodiments, the coding sequence encodes an amino acid sequence which is identical or at least 70%, 80%, 85%, 86%, 87%, 88%, 89%, 90%, 91%, 92%, 93%, 94%, 95%, 96%, 97%, 98%, or 99% identical to any one of SEQ ID NOs: 177-186, 247-256, 498-520, 1277, or an immunogenic fragment or immunogenic variant thereof. In some embodiments, the coding sequence comprises a nucleic acid sequences which is identical or at least 70%, 80%, 85%, 86%, 87%, 88%, 89%, 90%, 91%, 92%, 93%, 94%, 95%, 96%, 97%, 98%, or 99% identical to the sequences according to any one of SEQ ID NOs: 187-246, 257-316, 523-545, 548-570, 573-595, 598-620, 623-645, 648-670, or a fragment or a variant thereof.

In some embodiments, the coding sequence comprises at least one modified nucleotide selected from pseudouridine (ψ) and N1-methylpseudouridine (m1ψ), optionally wherein essentially all uracil nucleotides are replaced by pseudouridine (ψ) nucleotides and/or N1-methylpseudouridine (m1ψ) nucleotides. In some embodiments, the coding sequence is a codon modified coding sequence, wherein the amino acid sequence encoded by the at least one codon modified coding sequence is optionally not being modified compared to the amino acid sequence encoded by the corresponding wild type coding sequence, optionally wherein the at least one codon modified coding sequence is selected from C maximized coding sequence, CAI maximized coding sequence, human codon usage adapted coding sequence, G/C content modified coding sequence, and G/C optimized coding sequence, or any combination thereof.

In one embodiment, the coding RNA is an mRNA, optionally comprising or consisting of a nucleic acid sequence which is identical or at least 70%, 80%, 85%, 86%, 87%, 88%, 89%, 90%, 91%, 92%, 93%, 94%, 95%, 96%, 97%, 98%, or 99% identical to a nucleic acid sequence according to any one of SEQ ID NOs: 673-695, 698-720, 723-745, 748-770, 773-795, 798-820, 823-845, 848-870, 873-895, 898-920, 923-945, 948-970, 973-995, 998-1020, 1023-1045, 1048-1070, 1073-1095, 1098-1120, 1123-1145, 1148-1170, 1173-1195, 1198-1220, 1223-1245, 1248-1276 or a fragment or variant thereof.

In a second aspect, it is provided a pharmaceutical composition comprising the coding RNA of the disclosure. In some embodiments, the pharmaceutical composition further comprises lipid-base carriers, wherein the lipid-base carriers are lipid nanoparticles (LNP).

In a third aspect, it is provided a vaccine comprising the coding RNA or the pharmaceutical composition of the disclosure.

In a fourth aspect, it is provided a Kit or kit of parts, comprising the coding RNA, the pharmaceutical composition, and/or the vaccine of the disclosure, optionally comprising a liquid vehicle for solubilising, and, optionally, technical instructions providing information on administration and dosage of the components.

In a further aspect, it is provided a coding RNA, a pharmaceutical composition, the vaccine, or the kit or kit of parts of the disclosure, for use as a medicament. In one embodiment, the coding RNA, pharmaceutical composition, vaccine, or kit or kit of parts of the disclosure are for use in treating or preventing one or more symptoms associated with urinary tract infections (UTI) in a subject in need thereof.

In a further aspect, it is provided a coding RNA, a pharmaceutical composition, the vaccine, or the kit or kit of parts of the disclosure, for use as a medicament. In one embodiment, the coding RNA, pharmaceutical composition, vaccine, or kit or kit of parts of the disclosure are for use in treating or preventing a disease caused by E. coli.

In a further aspect, it is provided a method of treating or preventing a disorder, wherein the method comprises administering to a subject in need thereof an effective amount of the coding RNA, the pharmaceutical composition, the vaccine, or the kit or kit of parts of the disclosure. In one embodiment the method elicits antibodies which are capable of inhibiting bacterial adhesion.

### BRIEF DESCRIPTION OF THE DRAWINGS

FIG. 1: (A) (B): shows that mRNA constructs encoding different E. coli FimH antigen designs were expressed and partially secreted by mammalian cells using Western blot analysis. The experiment was performed as described in Example 2.1.

FIG. 2: (A)-(F) shows that formulated mRNA constructs encoding different E. coli FimH antigen designs elicited humoral immune response in mice. Serum and urine IgG titers were assessed by ELISA as described in Example 2.2.

FIG. 3: shows CD4+ and CD8+ T cell responses elicited by vaccination of mice with mRNA constructs encoding different E. coli FimH antigen designs as described in Example 2.4.

FIG. 4: (A)-(C): shows a dose response of formulated mRNA constructs encoding different E. coli FimH antigen designs upon vaccination of rats. Serum and urine IgG titers were assessed by ELISA as described in Example 3.1.

FIG. 5: (A) (B): shows that mRNA constructs encoding E. coli FimH antigen designs comprising uridine, ψ, or m1ψ were expressed and partially secreted by mammalian cells using Western blot analysis. The experiment was performed as described in Example 4.1.

FIG. 6: (A)-(C): shows that formulated mRNA constructs encoding E. coli FimH antigen designs comprising uridine, ψ, or m1ψ elicited humoral immune response in rats. Serum and urine IgG titers were assessed by ELISA as described in Example 2.2.

### DEFINITIONS

For the sake of clarity and readability the following definitions are provided. Any technical feature mentioned for these definitions may be read on each and every embodiment of the invention. Additional definitions and explanations may be specifically provided in the context of these embodiments.

Percentages in the context of numbers should be understood as relative to the total number of the respective items. In other cases, and unless the context dictates otherwise, percentages should be understood as percentages by weight (wt.-%).

About: The term “about” is used when determinants or values do not need to be identical, i.e. 100% the same. Accordingly, “about” means, that a determinant or values may diverge by 1% to 20%, for example by 1% to 10%; in particular, by 1%, 2%, 3%, 4%, 5%, 6%, 7%, 8%, 9%, 10%, 11%, 12%, 13%, 14%, 15%, 16%, 17%, 18%, 19%, 20%. The skilled person knows that e.g. certain parameters or determinants can slightly vary based on the method how the parameter has been determined. For example, if a certain determinants or value is defined herein to have e.g. a length of “about 100 nucleotides”, the length may diverge by 1% to 20%. Accordingly, the skilled person knows that in that specific example, the length may diverge by 1 to 20 nucleotides. Accordingly, a length of “about 100 nucleotides” may encompass sequences ranging from 80 to 120 nucleotides.

Adaptive immune response: The term “adaptive immune response” as used herein will be recognized and understood by the person of ordinary skill in the art, and is e.g. intended to refer to an antigen-specific response of the immune system (the adaptive immune system). Antigen specificity allows for the generation of responses that are tailored to specific pathogens or pathogen-infected cells. The ability to mount these tailored responses is usually maintained in the body by “memory cells” (B-cells).

Antigen: The term “antigen” as used herein will be recognized and understood by the person of ordinary skill in the art, and is e.g. intended to refer to a substance which may be recognized by the immune system, for example by the adaptive immune system, and is capable of triggering an antigen-specific immune response, e.g. by formation of antibodies and/or antigen-specific T cells as part of an adaptive immune response. Typically, an antigen may be or may comprise a peptide or protein which may be presented by the MHC to T-cells. Also fragments, variants and derivatives of peptides or proteins comprising at least one epitope are understood as antigens.

Antigenic peptide, polypeptide or protein: The term “antigenic peptide or protein” or “immunogenic peptide or protein” will be recognized and understood by the person of ordinary skill in the art, and is e.g. intended to refer to a peptide, protein derived from a (antigenic or immunogenic) protein which stimulates the body's adaptive immune system to provide an adaptive immune response. Therefore an antigenic/immunogenic peptide or protein comprises at least one epitope (as defined herein) or antigen (as defined herein) of the protein it is derived from.

Cationic: Unless a different meaning is clear from the specific context, the term “cationic” means that the respective structure bears a positive charge, either permanently or not permanently, but in response to certain conditions such as pH. Thus, the term “cationic” covers both “permanently cationic” and “cationisable”. The term “permanently cationic” means, e.g., that the respective compound, or group, or atom, is positively charged at any pH value or hydrogen ion activity of its environment. Typically, the positive charge results from the presence of a quaternary nitrogen atom. Where a compound carries a plurality of such positive charges, it may be referred to as permanently polycationic.

Cationisable: The term “cationisable” as used herein means that a compound, or group or atom, is positively charged at a lower pH and uncharged at a higher pH of its environment. Also in non-aqueous environments where no pH value can be determined, a cationisable compound, group or atom is positively charged at a high hydrogen ion concentration and uncharged at a low concentration or activity of hydrogen ions. It depends on the individual properties of the cationisable or polycationisable compound, in particular the pKa of the respective cationisable group or atom, at which pH or hydrogen ion concentration it is charged or uncharged. In diluted aqueous environments, the fraction of cationisable compounds, groups or atoms bearing a positive charge may be estimated using the so-called Henderson-Hasselbalch equation which is well-known to a person skilled in the art. E.g., in some embodiments, if a compound or moiety is cationisable, it is suitable that it is positively charged at a pH value of about 1 to 9, preferably 4 to 9, 5 to 8 or even 6 to 8, for example of a pH value of or below 9, of or below 8, of or below 7, for example at physiological pH values, e.g. about 7.3 to 7.4, i.e. under physiological conditions, particularly under physiological salt conditions of the cell in vivo. In other embodiments, it is suitable that the cationisable compound or moiety is predominantly neutral at physiological pH values, e.g. about 7.0-7.4, but becomes positively charged at lower pH values. In some embodiments, the range of pKa for the cationisable compound or moiety is about 5 to about 7.

Coding sequence/coding region: The terms “coding sequence” or “coding region” and the corresponding abbreviation “cds” as used herein will be recognized and understood by the person of ordinary skill in the art, and are e.g. intended to refer to a sequence of several nucleotide triplets, which may be translated into a peptide or protein. A coding sequence in the context of the present disclosure may be an RNA sequence consisting of a number of nucleotides that may be divided by three, which starts with a start codon and which for example terminates with a stop codon.

Derived from: The term “derived from” as used throughout the present specification in the context of a nucleic acid, i.e. for a nucleic acid “derived from” (another) nucleic acid, means that the nucleic acid, which is derived from (another) nucleic acid, shares e.g. at least 60%, 70%, 80%, 81%, 82%, 83%, 84%, 85%, 86%, 87%, 88%, 89%, 90%, 91%, 92%, 93%, 94%, 95%, 96%, 97%, 98%, or 99% sequence identity with the nucleic acid from which it is derived. The skilled person is aware that sequence identity is typically calculated for the same types of nucleic acids, i.e. for DNA sequences or for RNA sequences. Thus, it is understood, if a DNA is “derived from” an RNA or if an RNA is “derived from” a DNA, in a first step the RNA sequence is converted into the corresponding DNA sequence (in particular by replacing the uracils (U) by thymines (T) throughout the sequence) or, vice versa, the DNA sequence is converted into the corresponding RNA sequence (in particular by replacing the T by U throughout the sequence). Thereafter, the sequence identity of the DNA sequences or the sequence identity of the RNA sequences is determined. For example, a nucleic acid “derived from” a nucleic acid also refers to nucleic acid, which is modified in comparison to the nucleic acid from which it is derived, e.g. in order to increase RNA stability even further and/or to prolong and/or increase protein production. In the context of amino acid sequences (e.g. antigenic peptides or proteins) the term “derived from” means that the amino acid sequence, which is derived from (another) amino acid sequence, shares e.g. at least 60%, 70%, 75%, 80%, 81%, 82%, 83%, 84%, 85%, 86%, 87%, 88%, 89%, 90%, 91%, 92%, 93%, 94%, 95%, 96%, 97%, 98%, or 99% sequence identity with the amino acid sequence from which it is derived.

Donor strand peptide: The term “donor strand peptide” as used throughout the present specification means the portion of the FimC or FimG polypeptides that interacts in vivo or in vitro with FimHP and completes the atypical Ig-fold of FimHP by occupying the groove and running parallel to the subunit C-terminal F strand.

Fragment: The term “fragment” as used throughout the present specification in the context of a nucleic acid sequence (e.g. RNA or a DNA) or an amino acid sequence may typically be a shorter portion of a full-length sequence of e.g. a nucleic acid sequence or an amino acid sequence. Accordingly, a fragment typically consists of a sequence that is identical to the corresponding stretch within the full-length sequence. A particular fragment of a sequence in the context of the present disclosure, consists of a continuous stretch of entities, such as nucleotides or amino acids corresponding to a continuous stretch of entities in the molecule the fragment is derived from, which represents at least 40%, 50%, 60%, 70%, 80%, 90%, 95% of the total (i.e. full-length) molecule from which the fragment is derived (e.g. a virus protein). The term “fragment” as used throughout the present specification in the context of proteins or peptides may, typically, comprise a sequence of a protein or peptide as defined herein, which is, with regard to its amino acid sequence, N-terminally and/or C-terminally truncated compared to the amino acid sequence of the original protein. The term “fragment” as used throughout the present specification in the context of RNA sequences may, typically, comprise an RNA sequence that is 5′-terminally and/or 3′-terminally truncated compared to the reference RNA sequence. Such truncation may thus occur either on the amino acid level or correspondingly on the nucleic acid level. A sequence identity with respect to such a fragment as defined herein may therefore for example refer to the entire protein or peptide as defined herein or to the entire (coding) nucleic acid molecule of such a protein or peptide. Fragments of proteins or peptides may comprise at least one epitope of those proteins or peptides.

Identity (of a sequence): The term “identity” as used throughout the present specification in the context of a nucleic acid sequence or an amino acid sequence will be recognized and understood by the person of ordinary skill in the art, and is e.g. intended to refer to the percentage to which two sequences are identical. To determine the percentage to which two sequences are identical, e.g. nucleic acid sequences or amino acid (aa) sequences as defined herein, for example the aa sequences encoded by the nucleic acid sequence as defined herein or the aa sequences themselves, the sequences can be aligned in order to be subsequently compared to one another. Therefore, e.g. a position of a first sequence may be compared with the corresponding position of the second sequence. If a position in the first sequence is occupied by the same residue as is the case at a position in the second sequence, the two sequences are identical at this position. If this is not the case, the sequences differ at this position. If insertions occur in the second sequence in comparison to the first sequence, gaps can be inserted into the first sequence to allow a further alignment. If deletions occur in the second sequence in comparison to the first sequence, gaps can be inserted into the second sequence to allow a further alignment. The percentage to which two sequences are identical is then a function of the number of identical positions divided by the total number of positions including those positions which are only occupied in one sequence. The percentage to which two sequences are identical can be determined using an algorithm, e.g. an algorithm integrated in the BLAST program. Sequence identity can be determined by using the EMBOSS Water sequence alignment tool at the EMBL-EBI website https://www.ebi.ac.uk/Tools/psa/emboss_water/with the parameters gap open=12, gap extend=1 and matrix=BLOSUM62 for protein sequences or matrix=fullDNA for DNA/RNA sequences, or by using the EMBOSS Needle sequence alignment tool at the EMBL-EBI website https://www.ebi.ac.uk/Tools/psa/emboss_needle/with default parameters (e.g. gap open=10, gap extend=0.5, end gap penalty=false, end gap open=10 and end gap extend=0.5 and matrix=BLOSUM62 for protein sequences or matrix=fullDNA for DNA/RNA sequences). Unless specified otherwise, where the application refers to sequence identity to a particular reference sequence, the identity is intended to be calculated over the entire length of that reference sequence.

Immunogen, Immunogen: The terms “immunogen” or “immunogenic” will be recognized and understood by the person of ordinary skill in the art, and are e.g. intended to refer to a compound that is able to stimulate/induce an (adaptive) immune response. An immunogen may be a peptide, polypeptide, or protein.

Immune response: The term “immune response” will be recognized and understood by the person of ordinary skill in the art, and is e.g. intended to refer to a specific reaction of the adaptive immune system to a particular antigen (so called specific or adaptive immune response) or an unspecific reaction of the innate immune system (so called unspecific or innate immune response), or a combination thereof.

Lipidoid: A lipidoid, also referred to as lipidoid compound, is a lipid-like compound, i.e. an amphiphilic compound with lipid-like physical properties. In the context of the present disclosure, the term lipid is considered to encompass lipidoid compounds.

Nucleic acid, nucleic acid molecule: The terms “nucleic acid” or “nucleic acid molecule” as used herein, will be recognized and understood by the person of ordinary skill in the art. The terms “nucleic acid” or “nucleic acid molecule” particularly refers to DNA (molecules) or RNA molecules). The term is used synonymously with the term polynucleotide. For example, a nucleic acid or a nucleic acid molecule is a polymer comprising or consisting of nucleotide monomers that are covalently linked to each other by phosphodiester-bonds of a sugar/phosphate-backbone. The terms “nucleic acid” or “nucleic acid molecule” also encompasses modified nucleic acid (molecules), such as base-modified, sugar-modified or backbone-modified DNA or RNA (molecules) as defined herein.

Nucleic acid sequence, DNA sequence, RNA sequence: The terms “nucleic acid sequence”, “DNA sequence”, “RNA sequence” will be recognized and understood by the person of ordinary skill in the art, and e.g. refer to a particular and individual order of the succession of its nucleotides.

RNA species: In the context of the disclosure, the term “RNA species” is not restricted to mean one single molecule but is understood to comprise an ensemble of essentially identical RNA molecules. Accordingly, it may relate to a plurality of essentially identical RNA molecules.

RNA: The term “RNA” is the usual abbreviation for ribonucleic acid. It is a nucleic acid molecule, i.e. a polymer consisting of nucleotide monomers. These nucleotides are usually adenosine-monophosphate (AMP), uridine-monophosphate (UMP), guanosine-monophosphate (GMP) and cytidine-monophosphate (CMP) monomers or analogs thereof, which are connected to each other along a so-called backbone. The backbone is typically formed by phosphodiester bonds between the sugar, i.e. ribose, of a first and a phosphate moiety of a second, adjacent monomer. The specific order of the monomers, i.e. the order of the bases linked to the sugar/phosphate-backbone, is called the RNA sequence. In general, RNA can be obtained by transcription of a DNA sequence, e.g. inside a cell or in vitro. In the context of the disclosure, the RNA may be obtained by RNA in vitro transcription. Alternatively, RNA may be obtained by chemical synthesis.

RNA in vitro transcription: The terms “RNA in vitro transcription” or “in vitro transcription” relate to a process wherein RNA is synthesized in a cell-free system in vitro. RNA may be obtained by DNA-dependent in vitro transcription of an appropriate DNA template, which is typically a linear DNA template (e.g. linearized plasmid DNA or PCR product). The promoter for controlling RNA in vitro transcription can be any promoter for any DNA-dependent RNA polymerase. Particular examples of DNA-dependent RNA polymerases are the T7, T3, SP6, or Syn5 RNA polymerases. In one embodiment of the present invention the DNA template is linearized with a suitable restriction enzyme before it is subjected to RNA in vitro transcription. Reagents typically used in RNA in vitro transcription include: a DNA template (linearized plasmid DNA or PCR product) with a promoter sequence that has a high binding affinity for its respective RNA polymerase such as bacteriophage-encoded RNA polymerases (T7, T3, SP6, or Syn5); ribonucleotide triphosphates (NTPs) for the four bases (adenine, cytosine, guanine and uracil); optionally, a cap analogue as defined herein; optionally, modified nucleotides as defined herein; a DNA-dependent RNA polymerase capable of binding to the promoter sequence within the DNA template (e.g. T7, T3, SP6, or Syn5 RNA polymerase); optionally, a ribonuclease (RNase) inhibitor to inactivate any potentially contaminating RNase; optionally, pyrophosphatase; MgCl₂; a buffer (TRIS or HEPES) to maintain a suitable pH value, which can also contain antioxidants (e.g. DTT), and/or polyamines such as spermidine.

Variant (of a sequence): The term “variant” as used throughout the present specification in the context of a nucleic acid sequence will be recognized and understood by the person of ordinary skill in the art, and is e.g. intended to refer to a variant of a nucleic acid sequence derived from another nucleic acid sequence. E.g., a variant of a nucleic acid sequence may exhibit one or more nucleotide deletions, insertions, additions and/or substitutions compared to the nucleic acid sequence from which the variant is derived. A variant of a nucleic acid sequence may at least 50%, 60%, 70%, 80%, 90%, or 95% identical to the nucleic acid sequence the variant is derived from. The variant is a functional variant in the sense that the variant has retained at least 50%, 60%, 70%, 80%, 90%, or 95% or more of the function of the sequence where it is derived from. A “variant” of a nucleic acid sequence may have at least 70%, 75%, 80%, 85%, 90%, 95%, 98% or 99% nucleotide identity over a stretch of at least 10, 20, 30, 50, 75 or 100 nucleotide of such nucleic acid sequence.

The term “variant” as used throughout the present specification in the context of proteins or peptides is e.g. intended to refer to a proteins or peptide variant having an amino acid sequence which differs from the original sequence in one or more mutation(s)/substitution(s), such as one or more substituted, inserted and/or deleted amino acid(s). Suitably, these fragments and/or variants have the same, or a comparable specific antigenic property (immunogenic variants, antigenic variants). Insertions and substitutions are possible, in particular, at those sequence positions which cause no modification to the three-dimensional structure or do not affect the binding region. Modifications to a three-dimensional structure by insertion(s) or deletion(s) can easily be determined e.g. using CD spectra (circular dichroism spectra). A “variant” of a protein or peptide may have at least 70%, 75%, 80%, 85%, 90%, 95%, 98% or 99% amino acid identity over a stretch of at least 10, 20, 30, 50, 75 or 100 amino acids of such protein or peptide. Alternatively, a “variant” of a protein or polypeptide may have from 1 to 20, for example from 1 to 10 single amino acid mutations compared to such protein or peptide, for example, 1, 2, 3, 4, 5, 6, 7, 8, 9, 10, 11, 12, 13, 15, 16, 17, 18, 19 or 20 single amino acid mutations. For mutations we mean or include substitution, insertion or deletion. In one embodiment, a variant of a protein comprises a functional variant of the protein, which means, in the context of the disclosure, that the variant exerts essentially the same, or at least 40%, 50%, 60%, 70%, 80%, 90% of the immunogenicity as the protein it is derived from.

### DETAILED DESCRIPTION OF THE INVENTION

Where reference is made to “SEQ ID NOs” of other patent applications or patents, said sequences, e.g. amino acid sequences or nucleic acid sequences, are explicitly incorporated herein by reference. For “SEQ ID NOs” provided herein, information provided under “feature key”, i.e. “source” (for nucleic acids or proteins) or “misc_feature” (for nucleic acids) or “REGION” (for proteins) (in the sequence listing according to WIPO ST.26 Standard) is also explicitly included herein in its entirety. Where reference is made to “SEQ ID NOs” in the context of RNA sequences, the skilled person will understand and be able to derive RNA sequences from the referenced SEQ ID NOs also in cases where DNA sequences are provided. Where reference is made to “SEQ ID NOs” in the context of DNA sequences, the skilled person will understand and will be able to derive respective DNA sequences from the referenced SEQ ID NOs also in cases where RNA sequences are provided.

The inventors overcame the challenges of production of recombinant polypeptides of E. coli by administering an RNA vaccine encoding an antigenic polypeptide which is or is derived from E. coli FimH. The inventors further overcame the challenges of raising a rapid and robust immune response against E. coli FimH.

1: RNA Encoding an Antigenic Polypeptide of E. coli

In a first aspect, it is provided a coding RNA comprising at least one untranslated region (UTR); and at least one coding sequence encoding an antigenic polypeptide which is selected or derived from Escherichia coli type 1 fimbriae D-mannose specific adhesin (FimH).

It has to be noted that specific features and embodiments that are described in the context of the first aspect of the disclosure, that is the RNA of the disclosure, are likewise applicable to the second aspect (composition of the disclosure), the third aspect (vaccine of the disclosure), the fourth aspect (kit or kit of parts of the disclosure), or further aspects including medical uses and method of treatments.

The term “coding RNA” as used herein will be recognized and understood by the person of ordinary skill in the art, and are e.g. intended to refer to an RNA comprising a coding sequence (“cds”) comprising several nucleotide triplets, wherein said cds may be translated into a peptide or protein (e.g. upon administration to a cell or an organism).

The E. coli FimH of the disclosure may be selected or derived from any strain of Escherichia coli, such as any one of strains E. coli J96, E. coli 536, E. coli CFT073, E. coli UMN026, E. coli CLONE D i14, E. coli CLONE D i2, E. coli IA139, E. coli NA114, E. coli IHE3034, E. coli 789, E. coli F11 and E. coli UTI89.

In one embodiment, the E. coli FimH of the disclosure comprises or consists of or is derived from the amino acid sequence of SEQ ID NOs: 177-186, 247-256. In one embodiment, the E. coli FimH comprises an amino acid sequence which is identical or at least 70%, 80%, 85%, 86%, 87%, 88%, 89%, 90%, 91%, 92%, 93%, 94%, 95%, 96%, 97%, 98%, or 99% identical to any one of SEQ ID NOs: 177-186, 247-256. In one embodiment, the E. coli FimH is an immunogenic fragment or immunogenic variant of SEQ ID NOs: 177-186, 247-256. In one embodiment, the E. coli FimH comprises an amino acid sequence which is identical or at least 70%, 80%, 85%, 86%, 87%, 88%, 89%, 90%, 91%, 92%, 93%, 94%, 95%, 96%, 97%, 98%, or 99% identical to SEQ ID NO: 177. In one embodiment, the E. coli FimH comprises an amino acid sequence which is identical or at least 70%, 80%, 85%, 86%, 87%, 88%, 89%, 90%, 91%, 92%, 93%, 94%, 95%, 96%, 97%, 98%, or 99% identical to SEQ ID NO: 247.

In some embodiments, glycosylation sites in the encoded amino acid sequence are mutated/substituted which means that encoded amino acids which may be glycosylated, e.g. after translation of the coding RNA upon in vivo administration, are exchanged to a different amino acid. Accordingly, on nucleic acid level, codons encoding amino acids which are known or predicted to be N-glycosylated or O-glycosylated are substituted with amino acids unsusceptible or less susceptible to glycosylation, e.g., serine (S, Ser), aspartic acid (D, Asp), alanine (A, Ala) or glutamine (Q, Gln).

N- and/or O-glycosylation can be determined using any suitable means known in the art, for example, using the NetNGlyc 1.0 and NetOGlyc 4.0 Server (accessible at https://services.healthtech.dtu.dk/service.php?NetOGlyc-4.0 and https://services.healthtech.dtu.dk/service.php?NetOGlyc-4.0) using default settings.

In one embodiment, the antigenic polypeptide does not comprise (or is modified to not comprise) a glycosylation site at one of more of the positions selected from the group consisting of: positions 28, 91, 228, 249, and 256 relative to SEQ ID NO: 177 or at the positions of SEQ ID NOs: 178-186, 247-256 corresponding to those positions of SEQ ID NO: 177. In one embodiment, the polypeptide includes one or more of the following amino acid substitutions relative to SEQ ID NO: 177: N28S, N91D, N249D, N256D, or at the positions of SEQ ID NOs: 178-186, 247-256 corresponding to those positions of SEQ ID NO: 177, for example, one, two, three or four of those amino acid substitutions.

In various embodiments, the antigenic polypeptide comprises at least one amino acid substitution or mutation to lock the FimH lectin domain in a low mannose binding affinity conformation. In some embodiments in that context, the antigenic polypeptide comprises an amino acid selected from the group consisting of valine (V, Val), isoleucine (I, lie), leucine (L, Leu), glycine (G, Gly), methionine (M, Met) and alanine (A, Ala) at a position corresponding to position 165 of SEQ ID NO: 177 or at the positions of SEQ ID NOs: 178-186, 247-256 corresponding to this position of SEQ ID NO: 177. In one embodiment, the polypeptide comprises a F165V substitution of SEQ ID NO: 177 or at the positions of SEQ ID NOs: 178-186, 247-256 corresponding to this position of SEQ ID NO: 177. Said mutation has been reported in WO2021144369, whose content is incorporated herein by reference, to lock the FimH lectin domain in a low mannose binding affinity conformation.

In some embodiments, the encoded FimH comprises one or more amino acid substitutions selected from the group consisting of: N28S, V48C, L55C, N91S, N249Q, N256D, F165V, of SEQ ID NO: 177 or at the positions of SEQ ID NOs: 178-186, 247-256 corresponding to these positions of SEQ ID NO: 177; for example the encoded FimH comprises the following amino acid substitutions: N28S, N91S, N249Q; N28S, N91S, N249Q, N256D; N28S, V48C, L55C, N91S, N249Q; or F165V; of SEQ ID NO: 177 or at the positions of SEQ ID NOs: 178-186, 247-256 corresponding to these positions of SEQ ID NO: 177.

In some embodiments, the encoded FimH comprises one or more amino acid substitutions at positions selected from the group consisting of: F1, P12, G14, G15, G16, A18, P26, V27, V28, Q32, N33, L34, V35, R60, S62, Y64, G65, L68, F71, T86, L107, Y108, L109, V112, S113, A115, G116, V118, A119, A127, L129, Q133, F144, V154, V155, V156, P157, T158, V163 of SEQ ID NO: 247 or at the positions of SEQ ID NOs: 177-186, 248-256 corresponding to these positions of SEQ ID NO: 247.

In some embodiments, the encoded FimH comprises one or more amino acid substitutions selected from the group consisting of: F1I; F1L; F1V; F1M; F1Y; F1W; P12C; G14C; G15A; G15P; G16A; G16P; A18C; P26C; V27A; V27C; V28C; Q32C; N33C; L34C; L34N; L34S; L34T; L34D; L34E; L34K; L34R; V35C; R60P; S62C; Y64C; G65A; L68C; F71C; T86C; L107C; Y108C; L109C; V112C; S113C; A115V; G116C; V118C; A119C; A119N; A119S; A119T; A119D; A119E; A119K; A119R; A127C; L129C; Q133K; F144C; V154C; V156C; P157C; T158C; V1631; and V1851 of SEQ ID NO: 247 or at the positions of SEQ ID NOs: 177-186, 248-256 corresponding to these positions of SEQ ID NO: 247, or any combination thereof.

In some embodiments, the encoded FimH comprises the amino acid substitutions selected from the group consisting of: G15A and G16A; P12C and A18C; G14C and F144C; P26C and V35C; P26C and V154C; P26C and V156C; V27C and L34C; V28C and N33C; V28C and P157C; Q32C and Y108C; N33C and L 109C; N33C and P157C; V35C and L 107C; V35C and L 109C; S62C and T86C; S62C and L 129C; Y64C and L68C; Y64C and A 127C; L68C and F71C; V112C and T158C; S113C and G116C; S113C and T158C; V118C and V156C; A119C and V155C; L34N and V27A; L34S and V27A; L34T and V27A; L34D and V27A; L34E and V27A; L34K and V27A; L34R and V27A; A119N and V27A; A119S and V27A; A119T and V27A; A119D and V27A; A119E and V27A; A119K and V27A; A119R and V27A; G15A and V27A; G16A and V27A; G15P and V27A; G16P and V27A; G15A, G16A, and V27A; G65A and V27A; V27A and Q133K; and G15A, G16A, V27A, and Q133K of SEQ ID NO: 247 or at the positions of SEQ ID NOs: 177-186, 248-256 corresponding to these positions of SEQ ID NO: 247.

In one embodiment, the encoded FimH comprises the amino acid substitutions G15A, G16A and V27A of SEQ ID NO: 247 or at the positions of SEQ ID NOs: 177-186, 248-256 corresponding to these positions of SEQ ID NO: 247.

### Specific Antigen Designs

According to various embodiments, the coding sequence of the RNA encodes an antigenic polypeptide which is selected or derived from Escherichia coli FimH as defined herein, and one or more further peptide or protein elements. In some embodiments, the one or more further peptide or protein element(s) is heterologous.

Suitably, the further peptide or protein element may stabilise FimH subunits and/or shield their interactive surfaces (e.g. via a donor strand peptide). Additionally, the further peptide or protein element may promote secretion of the encoded antigenic peptide or protein of the disclosure (e.g. via secretory signal sequences). Additionally, the further peptide or protein element may promote anchoring of the encoded antigenic peptide or protein of the disclosure in the plasma membrane (e.g. via transmembrane elements), or promote formation of antigen complexes (e.g. via multimerization domains or antigen clustering domains).

Suitably, the coding sequence additionally encodes one or more peptide or protein elements selected from a donor strand peptide, a signal peptide a helper epitope, an antigen clustering domain, or a transmembrane domain. In some embodiments, the coding sequence encodes one or more further peptide or protein elements and a peptide linker.

### Donor Strand Peptides

In some embodiments, the coding RNA encodes an antigenic protein which is selected or derived from Escherichia coli FimH, and additionally encodes a donor strand peptide.

In some embodiments, the coding sequences encodes the following elements in N-terminal to C-terminal direction: an antigenic polypeptide which is selected or derived from Escherichia coli FimH; and a donor strand peptide.

In one embodiment, the donor strand peptide comprises or consists of the amino acid sequence of SEQ ID NO: 338 or SEQ ID NO: 339 or a variant thereof. In one embodiment, the variant of SEQ ID NO: 338 or SEQ ID NO: 339 has from 1 to 5, such as 1, 2, 3 or 4 single amino acid mutations compared to SEQ ID NO: 338 or SEQ ID NO: 339.

In one embodiment, the donor strand peptide comprises or consists of SEQ ID NO: 338. It may be particularly suitable in the context of the disclosure that the donor strand peptide comprises or consists of SEQ ID NO: 338 so that the polypeptide of the disclosure is in a low mannose binding affinity conformation.

### Peptide Linkers

In protein constructs composed of several elements, the protein elements are often separated by peptide linkers, which may be beneficial because they allow for a proper folding of the individual elements and thereby the proper functionality of each element.

When used in the context of the present disclosure, such linkers are particularly useful when encoded by a nucleic acid encoding at least two protein elements, such as an antigenic polypeptide and at least one further peptide or protein element. In that case, the linker is typically located on the polypeptide chain in between the polypeptide of interest and the at least one further protein element. When the coding sequence encodes more than one further peptide or protein elements, a linker can be suitably located between individual further peptide or protein elements. On nucleic acid level, the coding sequence for such linker is typically placed in the reading frame, 5′ or 3′ to the coding sequence for the polypeptide or protein of interest, or placed between coding regions for individual peptide or protein elements.

In one embodiment, the peptide linker comprises or consists of from 2 to 20 amino acids, from 4 to 15 amino acids, or from 5 to 10 amino acids, for example, 3, 4, 5, 6, 7, 8, 9, 10, 11, 12, 13, 14, 15, 16, 17, 18, 19 or 20 amino acids. Peptide linkers are for example composed of small, non-polar (e.g. Glycine) or polar (e.g. Serine or Threonine) amino acids. The small size of these amino acids provides flexibility, and allows for mobility of the connecting functional domains, as described by Chen et al. (Adv Drug Deliv Reb. 2013; 65(10): 1357-1369). The incorporation of Serine (S, Ser) or Threonine (T, Thr) can maintain the stability of the linker in aqueous solutions by forming hydrogen bonds with the water molecules, and therefore reduces an interaction between the linker and the protein moieties. Rigid linkers generally maintain the distance between the protein domains and they may be based on helical structures and/or they have a sequence that is rich in proline.

A typical sequence of a flexible linker is composed of repeats of the amino acids Glycine (G, Gly) and Serine (S, Ser). For instance, the linker may have the following sequence: GS, GSG, SGG, SG, GGS, SGS, GSS, SSG. In some embodiments, the same sequence is repeated multiple times (e.g. two, three, four, five or six times) to create a longer linker. In other embodiments, a single amino acid residue such as S or G can be used as a linker.

On nucleic acid level, particularly RNA level, any nucleotide sequence moiety can be employed that encodes any of linker used in the present disclosure. Owing to the degenerated genetic code, in the case of most polypeptides of SEQ ID NOs: 352-358, more than one particular nucleic acid sequence is conceivable as encoding the respective polypeptide list. While each and every such nucleic acid may generally be used in the context of the present disclosure, it is suitable that the nucleic acid sequence that encodes the polypeptide sequence is selected such that its sequence is codon-optimized according to the general guidance provided in this specification.

In some embodiments, the coding sequence encodes an antigenic protein which is selected or derived from Escherichia coli FimH, a donor strand peptide, and a peptide linker.

In one embodiment, the coding sequence encodes the following elements in N-terminal to C-terminal direction: an antigenic polypeptide which is selected or is derived from Escherichia coli FimH; a peptide linker; and a donor strand peptide.

When the coding sequence encodes the following elements in N-terminal to C-terminal direction: an antigenic polypeptide which is selected or derived from Escherichia coli FimH; a peptide linker; and a donor strand peptide it is particularly suitable that the peptide linker comprises or consists of:
(i) PGDGN [SEQ ID NO: 352], or a variant or fusion thereof, or
(ii) GGGGSGG [SEQ ID NO: 353], or a variant or fusion thereof, or
(iii) GGGGSGGGGSGGGGS [SEQ ID NO: 354], or a variant or fusion thereof, or
(iv) SGG [SEQ ID NO: 355], or a variant or fusion thereof, or
(v) SGGM [SEQ ID NO: 356], or a variant or fusion thereof, or
(vi) GGSGGSGGSGGSGGG [SEQ ID NO: 357], or a variant or fusion thereof, or
(vii) GGSGGSGGSGGS [SEQ ID NO: 358], or a variant or fusion thereof.

In one embodiment, the peptide linker is a variant of any one of SEQ ID NOs: 352-358, optionally wherein the variant has from 1 to 5, such as 1, 2, 3 or 4 single amino acid mutations compared to SEQ ID NOs: 352-358.

In one embodiment the peptide linker comprises or consists of SEQ ID NO: 352. It may be particularly suitable in the context of the disclosure that peptide linker located between the antigenic polypeptide and the donor strand peptide comprises or consists of SEQ ID NO: 352 so that the polypeptide of the disclosure is locked in a low mannose binding affinity conformation.

### Antigenic Polypeptide Conformation

In one embodiment, the antigenic polypeptide which is selected or derived from E. coli FimH is in a low mannose binding affinity conformation or tense (T) state, for example has a mannose binding affinity of K_{d} of about, 100 μM, 200 μM, 300 μM, 400 μM, 500 μM, 600 μM, 700 μM, 800 μM, 900 μM, or 1 mM or has no detectable mannose binding affinity. In one embodiment, the mannose binding affinity is K_{d}≈300 μM or higher (i.e. has no detectable mannose binding affinity), as disclosed in Kisiela D I, et al. Proc Natl Acad Sci USA. 2013 Nov. 19; 110(47):19089-94 and Sauer M M, et al. J Am Chem Soc. 2019 Jan. 16; 141(2):936-944. It is known in the art that the high mannose binding affinity conformation or relaxed (R) state of FimH corresponds to a mannose binding affinity of for example, K_{d}<1.2 μM.

Mannose binding can be determined using any suitable means known in the art, for example, surface plasmon resonance may be used to verify binding, binding specificity and binding constants of FimH constructs with Man-BSA and Glc-BSA (negative control), see, for example Rabbani S, et al. J Biol Chem. 2018 Feb. 2; 293(5):1835-1849, which is incorporated by reference herein.

The conformation of FimH can also be assessed by measuring the binding of conformational antibodies, using any suitable means known in the art, for example, surface plasmon resonance. Exemplary antibodies are capable of recognising epitopes differently overlapping the mannose-binding pocket of FimH, for example antibodies binding to epitopes overlapping with the mannose binding pocket, for example epitopes limited to just one loop of the mannose-binding pocket.

Exemplary antibodies are those disclosed in WO2016183501, or in Kisiela D I, et al. Proc Natl Acad Sci USA. 2013 Nov. 19; 110(47):19089-94, Kisiela D I, et al. PLoS Pathog. 2015 May 14; 11(5):e1004857 and which are incorporated by reference herein. In one embodiment, the conformational antibody has a variable heavy chain (VH) sequence of SEQ ID NO: 173 and a variable light chain (VL) sequence of SEQ ID NO: 174. In one embodiment, the conformational antibody has a variable heavy chain (VH) sequence of SEQ ID NO: 175 and a variable light chain (VL) sequence of SEQ ID NO: 176.

### Signal Peptides

In some embodiments, the coding sequence of the disclosure encodes at least one antigenic polypeptide which is selected or derived from Escherichia coli FimH, and additionally encodes a signal peptide.

Suitably, the signal peptide is selected or derived from FimH, FimC, immunoglobulin Kappa (IgK), immunoglobulin E (IgE), tissue plasminogen activator (TPA or HsPLAT), or human serum albumin (HSA or HsALB), or MHC class I lymphocyte antigen (HLA-A2).

In some embodiments, the signal peptide is selected or derived from IgE, IgK, FimH, FimC, TPA, HSA, or HLA-A2, wherein the amino acid sequences of said signal peptides is identical or at least 70%, 80%, 85%, 86%, 87%, 88%, 89%, 90%, 91%, 92%, 93%, 94%, 95%, 96%, 97%, 98%, or 99% identical to any one of amino acid sequences SEQ ID NOs: 394-400, or fragment or variant of any of these.

In some embodiments, the signal peptide is heterologous. In some embodiments, the signal peptide is selected or derived from IgE or IgK, wherein the amino acid sequences of said signal peptides is identical or at least 70%, 80%, 85%, 86%, 87%, 88%, 89%, 90%, 91%, 92%, 93%, 94%, 95%, 96%, 97%, 98%, or 99% identical to any one of amino acid sequences SEQ ID NOs: 394, 395, or fragment or variant of any of these.

In embodiments where the coding sequence of the disclosure additionally encodes a signal peptide, it is particularly suitable to generate a fusion protein comprising an N-terminal signal peptide and a C-terminal peptide or protein which is selected or derived from Escherichia coli FimH, optionally comprising a peptide linker and a donor strand peptide, wherein said C-terminal peptide or protein which is selected or derived from Escherichia coli FimH is for example lacking an endogenous N-terminal secretory signal peptide, such as SEQ ID NOs: 247-256 and 1277.

Constructs comprising an N-terminal signal peptide may ideally improve the secretion of the Escherichia coli FimH (that is encoded by the coding RNA of the first aspect). Accordingly, improved secretion of the Escherichia coli FimH, upon administration of the coding RNA of the first aspect, may be advantageous for the induction of humoral immune responses against the encoded Escherichia coli FimH antigenic protein.

Further suitable signal peptides may be selected from the list of amino acid sequences according to SEQ ID NOs: 1-1115 and SEQ ID NO: 1728 of published PCT patent application WO2017081082, which is incorporated by reference herein, or fragments or variants of these sequences, wherein said secretory signal peptides are N-terminally fused to the antigenic polypeptide which is selected or derived from Escherichia coli FimH or an immunogenic fragment or immunogenic variant thereof, lacking the endogenous secretory signal sequence.

Suitable examples of constructs comprising an N-terminal signal sequence are SEQ ID NOs: 498-520. The corresponding nuclei acid sequences for each of the above listed constructs can be found in Table 1.

### Antigen Clustering Domains or Multimerization Domains

In various embodiments, the coding sequence of the disclosure encodes an antigenic polypeptide which is selected or derived from Escherichia coli FimH, and additionally encodes an antigen clustering domain or multimerization domain.

Suitably, the antigen clustering domain (multimerization domain) is selected or derived from ferritin or lumazine synthase (LS, LumSynth).

In embodiments where the coding sequence of the disclosure additionally encodes an antigen clustering domain, it is particularly suitable to generate a fusion protein comprising an antigenic polypeptide which is selected or derived from Escherichia coli FimH—optionally comprising a donor strand peptide and a (first) peptide linker—further comprising an antigen clustering domain, and optionally a (second) peptide linker. Constructs comprising an antigen clustering domain may enhance the antigen clustering and may therefore promote immune responses e.g. by multiple binding events that occur simultaneously between the clustered antigens and the host cell receptors (see further details in Lopez-Sagaseta, Jacinto, et al. “Self-assembling protein nanoparticles in the design of vaccines”. Computational and structural biotechnology journal 14 (2016):58-68). Additionally, such constructs may additionally comprise an N-terminal signal sequence (as defined above).

Lumazine synthase (LS, LumSynth) is an enzyme with particle-forming properties, present in a broad variety of organisms and involved in riboflavin biosynthesis. Jardine et al. reported their attempts to enhance the immunoreactivity of recombinant gp120 against HIV infection through the inclusion of Lumazine synthase (LS, LumSynth) for the optimization of vaccine candidates (Jardine, Joseph, et al. “Rational HIV immunogen design to target specific germline B cell receptors”. Science 340.6133 (2013):711-716). A construct comprising Lumazine synthase allows the formation of multimeric nanoparticles, particularly 60-mer nanoparticles, displaying the antigenic polypeptide and thus optimizes B-cell activation.

In some embodiments, lumazine-synthase is used to promote the antigen clustering and may therefore promote immune responses of the coding sequence encoding the E. coli FimH antigen.

In some embodiments, the antigen clustering domain (multimerization domain) is selected or derived from lumazine-synthase (LS, LumSynth), wherein the amino acid sequences of said antigen clustering domain is identical or at least 70%, 80%, 85%, 86%, 87%, 88%, 89%, 90%, 91%, 92%, 93%, 94%, 95%, 96%, 97%, 98%, or 99% identical to any one of amino acid sequences (SEQ ID NOs: 443, 444), a fragment or variant of any of these.

Ferritin is a protein whose main function is intracellular iron storage. Almost all living organisms produce ferritin which is made of 24 subunits that self-assemble in a quaternary structure with octahedral symmetry. Its properties to self-assemble into nanoparticles are well-suited to carry and expose antigens.

In some embodiments, ferritin is used to promote the antigen clustering and may therefore promote immune responses of the RNA encoding the E. coli FimH antigen.

In some embodiments, the antigen clustering domain (multimerization domain) is or is derived from ferritin wherein the amino acid sequences of said antigen clustering domain is identical or at least 70%, 80%, 85%, 86%, 87%, 88%, 89%, 90%, 91%, 92%, 93%, 94%, 95%, 96%, 97%, 98%, or 99% identical to any one of amino acid sequences (SEQ ID NO: 457-459), a fragment or variant of any of these.

In some embodiments, the coding sequence encodes the following elements in N-terminal to C-terminal direction: an optional signal peptide, an antigen clustering domain which is selected or derived from lumazine synthase or ferritin as defined herein, a (second) peptide linker, and an antigenic polypeptide which is selected or derived from E. coli FimH, optionally further comprising a (first) peptide linker, and a donor strand peptide as defined herein.

In some alternative embodiments, the coding sequence encodes the following elements in N-terminal to C-terminal direction: an optional signal peptide, an antigenic polypeptide which is selected or derived from E. coli FimH, optionally comprising a (first) peptide linker and a donor strand peptide, a (second) peptide linker, and an antigen clustering domain which is suitably selected or derived from lumazine synthase or ferritin as defined herein.

In one embodiment, the (first) peptide linker comprises or consists of any one of SEQ ID NOs: 352-354 or a variant thereof, optionally wherein the variant has from 1 to 5, such as 1, 2, 3 or 4 single amino acid mutations compared to SEQ ID NOs: 352-354. In one embodiment, the (first) peptide linker comprises or consists of SEQ ID NO: 352. In one embodiment, the (second) peptide linker comprises or consists of any one of SEQ ID NO: 355-358 or a variant thereof, optionally wherein the variant has from 1 to 5, such as 1, 2, 3 or 4 single amino acid mutations compared to SEQ ID NOs: 355-358. In one embodiment, the (second) peptide linker comprises or consists of SEQ ID NO: 355.

Suitable examples of constructs comprising a heterologous antigen clustering domain are SEQ ID NOs: 507-510, 512-520. The corresponding nucleic acid sequences for each of the above listed constructs can be found in Table 1.

In various embodiments, the coding sequence of the disclosure encodes an antigenic polypeptide which is selected or derived from Escherichia coli FimH, and additionally encodes a transmembrane domain. In one embodiment the transmembrane domain is heterologous. A heterologous transmembrane domain promotes membrane anchoring of the encoded E. coli FimH antigenic polypeptide, and may thereby enhance the immune response (in particular cellular immune responses).

Suitably, the transmembrane domain is or is derived from an influenza HA transmembrane domain, for example derived from an influenza A HA H1N1, more for example from H1N1/A/Netherlands/602/2009, HA, aa521-566, GenBankAcc. No.: ACQ45338.1, (SEQ ID NO: 478).

Further suitable transmembrane domains are derived from Human immunodeficiency virus 1, Env, aa19-35, BAF32550.1, AB253679.1; Human immunodeficiency virus 1, Env, aa515-536, BAF32550.1, AB253679.1; Human immunodeficiency virus 1, Env, aa680-702, BAF32550.1, AB253679.1; Equine infectious anemia virus, Env, aa450-472, AAC03762.1, AF016316.1; Equine infectious anemia virus, Env, aa614-636, AAC03762.1, AF016316.1; Equine infectious anemia virus, Env, aa798-819, AAC03762.1, AF016316.1; Murine leukemia virus, Env, aa601-623, AAA46526.1, M93052.1; Mouse mammary tumor virus, Env, aa457-479, BAA03768.1, D16249.1; Mouse mammary tumor virus, Env, aa624-646, NP_056883.1, NC_001503.1; Vesicular stomatitis virus, G, aa477-499, CAA24525.1, V01214.1; Rabies virus, G, aa460-479, AEV43288.1, JN234423.1 (Env: Envelope glycoprotein; G: glycoprotein).

In embodiments where the coding sequence of the disclosure additionally encodes a heterologous transmembrane domain, it is particularly suitable to generate a fusion protein comprising an N-terminal peptide or protein comprising an antigenic polypeptide which is selected or derived from Escherichia coli FimH, optionally comprising a donor strand peptide and a (first) peptide linker (as defined above) between the antigenic polypeptide and the donor strand peptide; and a C-terminal heterologous transmembrane domain, and optionally a (second) peptide linker (as defined above) between the N-terminal peptide and the C-terminal peptide. Constructs comprising heterologous transmembrane domain may promote membrane anchoring of the antigen and may therefore promote immune responses, in particular cellular immune responses, of the RNA encoding the antigenic polypeptide. Additionally, such constructs may additionally comprise an N-terminal secretory signal sequence (as defined above). Alternatively, the transmembrane domain may be in the N-terminus.

Further transmembrane elements/domains may be selected from the list of amino acid sequences according to SEQ ID NOs: 1228-1343 of the patent application WO2017081082, or fragments or variants of these sequences, which is incorporated by reference herein.

In some embodiments, the coding sequence encodes the following elements in N-terminal to C-terminal direction: a secretory signal peptide, an antigenic polypeptide which is selected or derived from Escherichia coli FimH, optionally comprising a (first) peptide linker and a donor strand peptide, a (second) peptide linker, and a heterologous transmembrane domain.

A suitable example of a construct comprising a heterologous transmembrane element is SEQ ID NO: 511. The corresponding nucleic acid sequence for the construct can be found in Table 1.

In various embodiments of the invention, the coding sequence encodes the following elements for example in N-terminal to C-terminal direction:
a) a signal peptide, the antigenic polypeptide as defined herein;
b) a signal peptide, the antigenic polypeptide as defined herein, a peptide linker, a donor strand peptide;
c) an antigen clustering domain, a peptide linker, the antigenic polypeptide as defined herein, a peptide linker, a donor strand peptide;
d) a signal peptide, an antigen clustering domain, a peptide linker, the antigenic polypeptide as defined herein, a peptide linker, a donor strand peptide;
e) a signal peptide, the antigenic polypeptide as defined herein, a peptide linker, a donor strand peptide, a peptide linker, an antigen clustering domain; or
f) a signal peptide, the antigenic polypeptide as defined herein, a peptide linker, a donor strand peptide, a peptide linker, a transmembrane domain.

In some embodiments, the coding sequence encodes the following elements for example in N-terminal to C-terminal direction: a signal peptide, the antigenic polypeptide as defined herein, a peptide linker, and a donor strand peptide; optionally wherein the signal peptide is selected from SEQ ID NOs: 394-400, optionally wherein the signal peptide is SEQ ID NO: 395; the antigenic polypeptide is selected from SEQ ID NOs: 247-256, optionally wherein the antigenic polypeptide is SEQ ID NO: 247; the peptide linker is selected from SEQ ID NOs: 352-354, optionally wherein the peptide linker is SEQ ID NO: 352; the donor strand peptide is selected from SEQ ID NOs: 338, 339, optionally wherein the donor strand peptide is SEQ ID NO: 338.

In some embodiments, the coding sequence encodes the following elements for example in N-terminal to C-terminal direction: a signal peptide, the antigenic polypeptide as defined herein, a (first) peptide linker, a donor strand peptide, a (second) peptide linker; and an antigen clustering domain; optionally wherein the signal peptide is selected from SEQ ID NOs: 394-400, optionally wherein the signal peptide is SEQ ID NO: 394; the antigenic polypeptide is selected from SEQ ID NOs: 247-256, optionally wherein the antigenic polypeptide is SEQ ID NO: 247; the (first) peptide linker is selected from SEQ ID NOs: 352-354, optionally wherein the (first) peptide linker is SEQ ID NO: 352; the donor strand peptide is selected from SEQ ID NOs: 338, 339, optionally wherein the donor strand peptide is SEQ ID NO: 338; the (second) peptide linker is selected from SEQ ID NOs: 355-358, optionally wherein the (second) peptide linker is SEQ ID NO: 355; the antigen clustering domain is selected from SEQ ID NOs: 443, 444, 457-459, optionally wherein the peptide linker is SEQ ID NOs: 444 or 459.

Suitable sequences as defined above are provided in Table 1. Therein, each row corresponds to a suitable sequence. Column A of Table 1 provides a short description of suitable antigen constructs. Column B of Table 1 provides protein (amino acid) SEQ ID NOs of respective antigen constructs. Column C of Table 1 provides SEQ ID NO of the corresponding wild type or reference nucleic acid coding sequences. Column D of Table 1 provides SEQ ID NO of the corresponding G/C optimized nucleic acid coding sequences (opt1, gc). Column E of Table 1 provides SEQ ID NO of the corresponding human codon usage adapted nucleic acid coding sequences (opt3, human). Column F of Table 1 provides SEQ ID NO of further codon optimized coding sequences (opt4, main or opt5, gc mod).

Notably, the description explicitly includes the information provided under “feature key”, i.e. “source” (for nucleic acids or proteins) or “misc_feature” (for nucleic acids) or “REGION” (for proteins) of the ST.26 sequence listing of the present application. RNA constructs comprising coding sequences of Table 1, e.g. mRNA sequences comprising the coding sequences of Table 1 are provided in Table 3.

TABLE 1
Sequences (amino acid sequences and coding sequences).

| row | A | B | C | D | E | F |
|---|---|---|---|---|---|---|
| 1 | Ec(J96)-FimC(1- | 496 | | 521, 546 | 571, 596 | 621, 646 |
| | 36)_Ec(J96)- | | | | | |
| | FimC(37-241); | | | | | |
| | construct #1 | | | | | |
| 2 | HslgE(1-18)_Ec(J96)- | 497 | | 522, 547 | 572, 597 | 622, 647 |
| | FimC(37-241); | | | | | |
| | construct #2 | | | | | |
| 3 | Ec(J96)-FimH(1-21)_ | 498 | | 523, 548 | 573, 598 | 623, 648 |
| | Ec(J96)-FimH(22-300); | | | | | |
| | construct #3 | | | | | |
| 4 | HslgE(1-18)_Ec(J96)- | 499 | | 524, 549 | 574, 599 | 624, 649 |
| | FimH(22-300); | | | | | |
| | construct #4 | | | | | |
| 5 | MmlgK(1-20)_Ec(J96)- | 500 | | 525, 550 | 575, 600 | 625, 650 |
| | FimH(22-300); | | | | | |
| | construct #5 | | | | | |
| 6 | Ec(J96)-FimH(1- | 501 | | 526, 551 | 576, 601 | 626, 651 |
| | 21)_Ec(J96)- | | | | | |
| | FimH(22-300)_Linker- | | | | | |
| | PGDGN_Ec(J96)- | | | | | |
| | FimG(24-37); | | | | | |
| | construct #6 | | | | | |
| 7 | HslgE(1-18)_Ec(J96)- | 502 | | 527, 552 | 577, 602 | 627, 652 |
| | FimH(22-300)_Linker- | | | | | |
| | PGDGN_Ec(J96)- | | | | | |
| | FimG(24-37); | | | | | |
| | construct #7 | | | | | |
| 8 | HslgE(1-18)_Ec(J96)- | 503 | | 528, 553 | 578, 603 | 628, 653 |
| | FimH(22-300)_Linker- | | | | | |
| | G4SGG_Ec(J96)- | | | | | |
| | FimG(24-37); | | | | | |
| | construct #8 | | | | | |
| 9 | MmlgK(1-20)_Ec(J96)- | 504 | | 529, 554 | 579, 604 | 629, 654 |
| | FimH(22-300)_Linker- | | | | | |
| | PGDGN_Ec(J96)- | | | | | |
| | FimG(24-37); | | | | | |
| | construct #9 | | | | | |
| 10 | MmlgK(1-20)_Ec(536)- | 505 | | 530, 555 | 580, 605 | 630, 655 |
| | FimH(22-300)_Linker- | | | | | |
| | PGDGN_Ec(J96)- | | | | | |
| | FimG(24-37); | | | | | |
| | construct #10 | | | | | |
| 11 | HslgE(1-18)_Ec(J96)- | 506 | | 531, 556 | 581, 606 | 631, 656 |
| | FimH(22-300)_Linker- | | | | | |
| | (G4S)3_Ec(J96)- | | | | | |
| | FimC(135-148); | | | | | |
| | construct #11 | | | | | |
| 12 | LumSynth(1-154_C37A_ | 507 | | 532, 557 | 582, 607 | 632, 657 |
| | N102D)_Linker(GGS)4- | | | | | |
| | GGG_Ec(J96)-FimH(22- | | | | | |
| | 300)_Linker- | | | | | |
| | PGDGN_Ec(J96)- | | | | | |
| | FimG(24-37); | | | | | |
| | construct #12 | | | | | |
| 13 | HslgE(1-18)_Ec(J96)- | 508 | | 533, 558 | 583, 608 | 633, 658 |
| | FimH(22-300)_Linker- | | | | | |
| | PGDGN_Ec(J96)- | | | | | |
| | FimG(24-37)_Linker- | | | | | |
| | SGG_LumSynth(1- | | | | | |
| | 154_C37A_N102D); | | | | | |
| | construct #13 | | | | | |
| 14 | HslgE(1-18)_Ec(J96)- | 509 | | 534, 559 | 584, 609 | 634, 659 |
| | FimH(22-300)_Linker- | | | | | |
| | PGDGN_Ec(J96)- | | | | | |
| | FimG(24-37)_Linker- | | | | | |
| | SGG_Ferritin(5- | | | | | |
| | 167_N19Q); | | | | | |
| | construct #14 | | | | | |
| 15 | MmlgK(1-20)_Ec(J96)- | 510 | | 535, 560 | 585, 610 | 635, 660 |
| | FimH(22-300)_Linker- | | | | | |
| | PGDGN_Ec(J96)- | | | | | |
| | FimG(24-37)_Linker- | | | | | |
| | (GGS)4_Ferritin(5-166); | | | | | |
| | construct #15 | | | | | |
| 16 | HslgE(1-18)_Ec(J96)- | 511 | | 536, 561 | 586, 611 | 636, 661 |
| | FimH(22-300)_Linker- | | | | | |
| | PGDGN_Ec(J96)- | | | | | |
| | FimG(24-37)_ | | | | | |
| | TMdomain- | | | | | |
| | HA(510-566); | | | | | |
| | construct #16 | | | | | |
| 17 | HslgE(1-18)_ | 512 | | 537, 562 | 587, 612 | 637, 662 |
| | LumSynth(1- | | | | | |
| | 154_C37A_N102D)_ | | | | | |
| | Linker(GGS)4- | | | | | |
| | GGG_Ec(J96)-FimH(22- | | | | | |
| | 300)_Linker- | | | | | |
| | PGDGN_Ec(J96)- | | | | | |
| | FimG(24-37); | | | | | |
| | construct #17 | | | | | |
| 18 | HslgE(1-18)_Ec(J96)- | 513 | | 538, 563 | 588, 613 | 638, 663 |
| | FimH(22-300_N28S_ | | | | | |
| | N91S_N249Q)_Linker- | | | | | |
| | PGDGN_Ec(J96)- | | | | | |
| | FimG(24-37)_Linker- | | | | | |
| | SGG_LumSynth(1- | | | | | |
| | 154_C37A_N102D); | | | | | |
| | construct #18 | | | | | |
| 19 | HslgE(1-18)_Ec(J96)- | 514 | | 539, 564 | 589, 614 | 639, 664 |
| | FimH(22-300_N28S_ | | | | | |
| | N91S_N249Q_N256D)_ | | | | | |
| | Linker-PGDGN_Ec(J96)- | | | | | |
| | FimG(24-37)_Linker- | | | | | |
| | SGG_LumSynth(1- | | | | | |
| | 154_C37A_N102D); | | | | | |
| | construct #19 | | | | | |
| 20 | HslgE(1-18)_Ec(J96)- | 515 | | 540, 565 | 590, 615 | 640, 665 |
| | FimH(22- | | | | | |
| | 300_N28S_V48C_ | | | | | |
| | L55C_N91S_N249Q)_ | | | | | |
| | Linker-PGDGN_Ec(J96)- | | | | | |
| | FimG(24-37)_Linker- | | | | | |
| | SGG_LumSynth(1- | | | | | |
| | 154_C37A_N102D); | | | | | |
| | construct #20 | | | | | |
| 21 | HslgE(1-18)_Ec(J96)- | 516 | | 541, 566 | 591, 616 | 641, 666 |
| | FimH(22-300_F165V)_ | | | | | |
| | Linker-PGDGN_ | | | | | |
| | Ec(J96)-FimG(24- | | | | | |
| | 37)_Linker-SGG_ | | | | | |
| | LumSynth(1- | | | | | |
| | 154_C37A_N102D); | | | | | |
| | construct #21 | | | | | |
| 22 | HslgE(1-18)_Ec(J96)- | 517 | | 542, 567 | 592, 617 | 642, 667 |
| | FimH(22-300_N28S_ | | | | | |
| | N91S_N249Q)_Linker- | | | | | |
| | PGDGN_Ec(J96)- | | | | | |
| | FimG(24-37)_Linker- | | | | | |
| | SGG_Ferritin(5- | | | | | |
| | 167_N19Q); | | | | | |
| | construct #22 | | | | | |
| 23 | HslgE(1-18)_Ec(J96)- | 518 | | 543, 568 | 593, 618 | 643, 668 |
| | FimH(22-300_ | | | | | |
| | N28S_N91S_ | | | | | |
| | N249Q_N256D)_Linker- | | | | | |
| | PGDGN_Ec(J96)- | | | | | |
| | FimG(24-37)_Linker- | | | | | |
| | SGG_Ferritin(5- | | | | | |
| | 167_N19Q); | | | | | |
| | construct #23 | | | | | |
| 24 | HslgE(1-18)_Ec(J96)- | 519 | | 544, 569 | 594, 619 | 644, 669 |
| | FimH(22- | | | | | |
| | 300_N28S_V48C_ | | | | | |
| | L55C_N91S_N249Q)_ | | | | | |
| | Linker-PGDGN_Ec(J96)- | | | | | |
| | FimG(24-37)_Linker- | | | | | |
| | SGG_Ferritin(5- | | | | | |
| | 167_N19Q); | | | | | |
| | construct #24 | | | | | |
| 25 | HslgE(1-18)_ | 520 | | 545,570 | 595, 620 | 645, 670 |
| | Ec(J96)-FimH(22- | | | | | |
| | 300_F165V)_Linker- | | | | | |
| | PGDGN_Ec(J96)- | | | | | |
| | FimG(24-37)_Linker- | | | | | |
| | SGG_Ferritin(5- | | | | | |
| | 167_N19Q); | | | | | |
| | construct #25 | | | | | |
| 26 | Ec(J96)-FimH | 177 | 187 | 197, 207 | 217 | 227, 237 |
| 27 | Ec(536)-FimH | 178 | 188 | 198, 208 | 218 | 228, 238 |
| 28 | Ec(CFT073)-FimH | 179 | 189 | 199, 209 | 219 | 229, 239 |
| 29 | Ec(IHE3034)-FimH | 180 | 190 | 200, 210 | 220 | 230, 240 |
| 30 | Ec(NA114)-FimH | 181 | 191 | 201, 211 | 221 | 231, 241 |
| 31 | Ec(F11)-FimH | 182 | 192 | 202, 212 | 222 | 232, 242 |
| 32 | Ec(J96)-FimH(N28S_ | 183 | 193 | 203, 213 | 223 | 233, 243 |
| | N91S_N249Q) | | | | | |
| 33 | Ec(J96)- | 184 | 194 | 204, 214 | 224 | 234, 244 |
| | FimH(N28S_N91S_ | | | | | |
| | N249Q_N256D) | | | | | |
| 34 | Ec(J96)- | 185 | 195 | 205, 215 | 225 | 235, 245 |
| | FimH(N28S_V48C_ | | | | | |
| | L55C_N91S_N249Q) | | | | | |
| 35 | Ec(J96)-FimH(F165V) | 186 | 196 | 206, 216 | 226 | 236, 246 |
| 36 | Ec(J96)-FimH(22-300) | 247 | 257 | 267, 277 | 287 | 297, 307 |
| 37 | Ec(536)-FimH(22-300) | 248 | 258 | 268, 278 | 288 | 298, 308 |
| 38 | Ec(CFT073)- | 249 | 259 | 269, 279 | 289 | 299, 309 |
| | FimH(22-300) | | | | | |
| 39 | Ec(IHE3034)- | 250 | 260 | 270, 280 | 290 | 300, 310 |
| | FimH(22-300) | | | | | |
| 40 | Ec(NA114)- | 251 | 261 | 271, 281 | 291 | 301, 311 |
| | FimH(22-300) | | | | | |
| 41 | Ec(F11)-FimH(22-300) | 252 | 262 | 272, 282 | 292 | 302, 312 |
| 42 | Ec(J96)-FimH(22- | 253 | 263 | 273, 283 | 293 | 303, 313 |
| | 300_N28S_N91S_ | | | | | |
| | N249Q) | | | | | |
| 43 | Ec(J96)-FimH(22- | 254 | 264 | 274, 284 | 294 | 304, 314 |
| | 300_N28S_N91S_ | | | | | |
| | N249Q_N256D) | | | | | |
| 44 | Ec(J96)-FimH(22-300_ | 255 | 265 | 275, 285 | 295 | 305, 315 |
| | N28S_V48C_L55C_ | | | | | |
| | N91S_N249Q) | | | | | |
| 45 | Ec(J96)-FimH(22- | 256 | 266 | 276, 286 | 296 | 306, 316 |
| | 300_F165V) | | | | | |
| 46 | Ec(J96)-FimC | 317 | 318 | 319, 320 | 321 | 322, 323 |
| 47 | Ec(J96)-FimC(37-241) | 324 | 325 | 326, 327 | 328 | 329, 330 |
| 48 | Ec(J96)-FimG | 331 | 332 | 333, 334 | 335 | 336, 337 |
| 49 | Ec(J96)-FimG(24-37); | 338 | 340 | 342, 344 | 346 | 348, 350 |
| | FimG donor | | | | | |
| | strand, G-peptide | | | | | |
| 50 | Ec(J96)-FimC(135-148); | 339 | 341 | 343, 345 | 347 | 349, 351 |
| | FimC donor | | | | | |
| | strand, C-peptide | | | | | |
| 51 | Linker-PGDGN | 352 | | 359, 366 | 373 | 380, 387 |
| 52 | Linker-G4SGG | 353 | | 360, 367 | 374 | 381, 388 |
| 53 | Linker-(G4S)3 | 354 | | 361, 368 | 375 | 382, 389 |
| 54 | Linker-SGG | 355 | | 362, 369 | 376 | 383, 390 |
| 55 | Linker-SGGM | 356 | | 363, 370 | 377 | 384, 391 |
| 56 | Linker-(GGS)4-GGG | 357 | | 364, 371 | 378 | 385, 392 |
| 57 | Linker-(GGS)4 | 358 | | 365, 372 | 379 | 386, 393 |
| 58 | SignalPeptide- | 394 | 401 | 408, 415 | 422 | 429, 436 |
| | HslgE(1-18) | | | | | |
| 59 | SignalPeptide- | 395 | 402 | 409, 416 | 423 | 430, 437 |
| | MmlgK(1-20) | | | | | |
| 60 | SignalPeptide-Ec(J96)- | 396 | 403 | 410, 417 | 424 | 431, 438 |
| | FimH(1-21) | | | | | |
| 61 | SignalPeptide-Ec(J96)- | 397 | 404 | 411, 418 | 425 | 432, 439 |
| | FimC(1-36) | | | | | |
| 62 | SignalPeptide- | 398 | 405 | 412, 419 | 426 | 433, 440 |
| | HsPLAT(1-22) | | | | | |
| 63 | SignalPeptide- | 399 | 406 | 413, 420 | 427 | 434, 441 |
| | HsALB(1-18) | | | | | |
| 64 | SignalPeptide-HsHLA- | 400 | 407 | 414, 421 | 428 | 435, 442 |
| | A2(1-24) | | | | | |
| 65 | LumSynth(1-154); | 443 | 445 | 447,449 | 451 | 453, 455 |
| | Aa(VF5)-ribH(1-154) | | | | | |
| 66 | LumSynth(1-154_ | 444 | 446 | 448, 450 | 452 | 454, 456 |
| | C37A_N102D); | | | | | |
| | Aa(VF5)-ribH(1- | | | | | |
| | 154_C37A_N102D) | | | | | |
| 67 | Ferritin(5-167); | 457 | 460 | 463, 466 | 469 | 472, 475 |
| | Hp(J99)-pfr(5-167) | | | | | |
| 68 | Ferritin(5-166); | 458 | 461 | 464, 467 | 470 | 473, 476 |
| | Hp(J99)-pfr(5-166) | | | | | |
| 69 | Ferritin(5-167_N19Q); | 459 | 462 | 465, 468 | 471 | 474, 477 |
| | Hp(J99)-pfr(5- | | | | | |
| | 167_N19Q) | | | | | |
| 70 | TMdomain-HA(510-566) | 478 | 490 | 491, 492 | 493 | 494, 495 |
| 71 | TMdomain-Env(19-35) | 479 | | | | |
| 72 | TMdomain-Env(515-536) | 480 | | | | |
| 73 | TMdomain-Env(680-702) | 481 | | | | |
| 74 | TMdomain-Env(450-472) | 482 | | | | |
| 75 | TMdomain-Env(614-636) | 483 | | | | |
| 76 | TMdomain-Env(798-819) | 484 | | | | |
| 77 | TMdomain-Env(601-623) | 485 | | | | |
| 78 | TMdomain-Env(457-479) | 486 | | | | |
| 79 | TMdomain-Env(624-646) | 487 | | | | |
| 80 | TMdomain-G(477-499) | 488 | | | | |
| 81 | TMdomain-G(460-479) | 489 | | | | |
| 82 | mAb-926_VH | 173 | | | | |
| 83 | mAb-926_VL(kappa) | 174 | | | | |
| 84 | mAb-475_VH | 175 | | | | |
| 85 | mAb-475_VL(kappa) | 176 | | | | |
| 86 | Ec(J96)-FimH(22- | 1277 | | | | |
| | 300_N28S_G36A_ | | | | | |
| | G37A_V48S_N91S_ | | | | | |
| | N249Q)_Linker- | | | | | |
| | GGSSGGG_Ec(J96)- | | | | | |
| | FimG(24-37) | | | | | |

Aa: Aquifex aeolicus; Ec: Escherichia coli; Env: Envelope glycoprotein; G: Glycoprotein; HA: Hemagglutinin; Hp: Helicobacter pylori; Hs: Homo sapiens; IgE: immunoglobulin E; IgK: immunoglobulin Kappa; LumSynth, LS: Lumazine synthase; Mm: Mus musculus; TMdomain, TM: transmembrane domain

### Suitable Coding Sequences:

According to some embodiments, the coding RNA of the disclosure comprises a coding sequence encoding an antigenic polypeptide which is selected or derived from an Escherichia coli FimH as defined herein, or fragments and variants thereof. In that context, any coding sequence encoding at least one antigenic protein as defined herein, or fragments and variants thereof may be understood as suitable coding sequence and may therefore be comprised in the nucleic acid of the disclosure.

In some embodiments, the coding RNA of the first aspect comprises or consists of a coding sequence encoding an antigenic polypeptide which is selected or derived from Escherichia coli FimH as defined herein, for example encoding any one of SEQ ID NOs: 177-186, 247-256, 498-520, 1277, or fragments of variants thereof. It has to be understood that, on RNA level, any sequence which encodes an amino acid sequences being identical or at least 70%, 80%, 85%, 86%, 87%, 88%, 89%, 90%, 91%, 92%, 93%, 94%, 95%, 96%, 97%, 98%, or 99% identical to any one of SEQ ID NOs: 177-186, 247-256, 498-520, 1277, or fragments or variants thereof, may be selected and may accordingly be understood as suitable coding sequence of the disclosure.

In some embodiments, the coding sequence encodes an amino acid sequence which is identical or at least 70%, 80%, 85%, 86%, 87%, 88%, 89%, 90%, 91%, 92%, 93%, 94%, 95%, 96%, 97%, 98%, or 99% identical to any one of SEQ ID NOs: 498-520, 1277 or an immunogenic fragment or immunogenic variant thereof.

In some embodiments, the coding sequence encodes an amino acid sequence which is identical or at least 90%, 91%, 92%, 93%, 94%, 95%, 96%, 97%, 98%, or 99% identical to any one of SEQ ID NOs: 504, 508, and 509, or an immunogenic fragment or immunogenic variant thereof.

In some embodiments, the coding RNA of the first aspect comprises a coding sequence that comprises at least one of the nucleic acid sequences being identical or at least 70%, 80%, 85%, 86%, 87%, 88%, 89%, 90%, 91%, 92%, 93%, 94%, 95%, 96%, 97%, 98%, or 99% identical to the sequences according to any one of SEQ ID NOs: 187-246, 257-316, 523-545, 548-570, 573-595, 598-620, 623-645, 648-670, or a fragment or variant of any of these sequences.

In some embodiments, the coding RNA of the first aspect comprises a coding sequence that comprises at least one of the nucleic acid sequences being identical or at least 70%, 80%, 85%, 86%, 87%, 88%, 89%, 90%, 91%, 92%, 93%, 94%, 95%, 96%, 97%, 98%, or 99% identical to the sequences according to any one of SEQ ID NOs: 523-545, 548-570, 573-595, 598-620, 623-645, 648-670, or a fragment or variant thereof.

In some embodiments, the coding RNA of the first aspect comprises a coding sequence that comprises at least one of the nucleic acid sequences being identical or at least 70%, 80%, 85%, 86%, 87%, 88%, 89%, 90%, 91%, 92%, 93%, 94%, 95%, 96%, 97%, 98%, or 99% identical to the sequences according to any one of SEQ ID NOs: 529, 533, 534, 554, 558, 559, 579, 583, 584, 604, 608, 609, 629, 633, 634, 654, 658, 659, or a fragment or variant of any of these sequences.

In some embodiments, the coding RNA of the first aspect is an artificial RNA.

The term “artificial RNA” as used herein is intended to refer to an RNA that does not occur naturally. In other words, an artificial RNA may be understood as a non-natural RNA molecule. Such RNA molecules may be non-natural due to its individual sequence (e.g. G/C content modified coding sequence, UTRs) and/or due to other modifications, e.g. structural modifications of nucleotides. Typically, artificial RNA may be designed and/or generated by genetic engineering to correspond to a desired artificial sequence of nucleotides. In this context, an artificial RNA is a sequence that may not occur naturally, i.e. a sequence that differs from the wild type or reference sequence/the naturally occurring sequence by at least one nucleotide (via e.g. codon modification as further specified below). The term “artificial RNA” is not restricted to mean “one single molecule” but is understood to comprise an ensemble or plurality of essentially identical RNA molecules.

In some embodiments, the coding RNA is a modified and/or stabilized RNA.

According to some embodiments, the coding RNA may thus be provided as a “stabilized RNA” that is to say an RNA showing improved resistance to in vivo degradation and/or an RNA showing improved stability in vivo, and/or an RNA showing improved translatability in vivo. The term “stabilized RNA” refers to an RNA that is modified such that it is more stable to disintegration or degradation, e.g., by environmental factors or enzymatic digest, such as by exo- or endonuclease degradation, compared to an RNA without such modification. In one embodiment, a stabilized RNA in the context of the present disclosure is stabilized in a cell, such as a prokaryotic or eukaryotic cell, such as in a mammalian cell, such as a human cell. The stabilization effect may also be exerted outside of cells, e.g. in a buffer solution etc., e.g., for storage of a composition comprising the stabilized RNA.

The coding RNA of the present disclosure may be provided as a “stabilized RNA”.

In the following, suitable modifications/adaptations are described that are capable of “stabilizing” the RNA.

In some embodiments, the coding RNA comprises at least one codon modified coding sequence.

In some embodiments, the at least one coding sequence of the coding RNA is a codon modified coding sequence. Suitably, the amino acid sequence encoded by the at least one codon modified coding sequence is not being modified compared to the amino acid sequence encoded by the corresponding wild type or reference coding sequence.

The term “codon modified coding sequence” relates to coding sequences that differ in at least one codon (triplets of nucleotides coding for one amino acid) compared to the corresponding wild type or reference coding sequence. Suitably, a codon modified coding sequence in the context of the disclosure may show improved resistance to in vivo degradation and/or improved stability in vivo, and/or improved translatability in vivo. Codon modifications in the broadest sense make use of the degeneracy of the genetic code wherein multiple codons may encode the same amino acid and may be used interchangeably to optimize/modify the coding sequence for in vivo applications.

In some embodiments, the coding sequence of the coding RNA is a codon modified coding sequence, wherein the codon modified coding sequence is selected from C maximized coding sequence, CAI maximized coding sequence, human codon usage adapted coding sequence, G/C content modified coding sequence, and G/C optimized coding sequence, or any combination thereof.

In some embodiments, the coding sequence of the coding RNA has a G/C content of at least about 50%, 55%, or 60%. In particular embodiments, the at least one coding sequence of the RNA has a G/C content of at least about 50%, 51%, 52%, 53%, 54%, 55%, 56%, 57%, 58%, 59%, 60%, 61%, 62%, 63%, 64%, 65%, 66%, 67%, 68%, 69%, 70%, 71% or 72%.

When transfected into mammalian host cells, the coding RNA comprising the codon modified coding sequence has a stability of between 12-18 hours, or greater than 18 hours, e.g., 24, 36, 48, 60, 72, or greater than 72 hours and is capable of being expressed by the mammalian host cell (e.g. a muscle cell). RNA detection methods are known in the art.

When transfected into mammalian host cells, the coding RNA comprising the codon modified coding sequence is translated into protein, wherein the amount of protein is at least comparable to, or for example at least 10% more than, or at least 20% more than, or at least 30% more than, or at least 40% more than, or at least 50% more than, or at least 100% more than, or at least 200% or more than the amount of protein obtained by a naturally occurring or wild type or reference coding sequence transfected into mammalian host cells.

In some embodiments, the coding RNA may be modified, wherein the C content of the at least one coding sequence may be increased, for example maximized, compared to the C content of the corresponding wild type or reference coding sequence (herein referred to as “C maximized coding sequence”). The generation of a C maximized nucleic acid sequences may suitably be carried out using a modification method according to WO2015062738. In this context, the disclosure of WO2015062738 is included herewith by reference.

In some embodiments, the coding RNA may be modified, wherein the G/C content of the at least one coding sequence may be optimized compared to the G/C content of the corresponding wild type or reference coding sequence (herein referred to as “G/C optimized coding sequence”). “Optimized” in that context refers to a coding sequence wherein the G/C content is for example increased to the essentially highest possible G/C content. The generation of a G/C content optimized nucleic acid sequence may be carried out using a method according to WO2002098443. In this context, the disclosure of WO2002098443 is included in its full scope in the present disclosure. G/C optimized coding sequences are indicated by the abbreviations “opt1” or “gc”.

In some embodiments, the coding RNA may be modified, wherein the codons in the at least one coding sequence may be adapted to human codon usage (herein referred to as “human codon usage adapted coding sequence”). Codons encoding the same amino acid occur at different frequencies in humans. Accordingly, the coding sequence of the RNA is modified such that the frequency of the codons encoding the same amino acid corresponds to the naturally occurring frequency of that codon according to the human codon usage. For example, in the case of the amino acid Ala, the wild type or reference coding sequence is for example adapted in a way that the codon “GCC” is used with a frequency of 0.40, the codon “GCT” is used with a frequency of 0.28, the codon “GCA” is used with a frequency of 0.22 and the codon “GCG” is used with a frequency of 0.10 etc. (see e.g. Table 2 of published PCT patent application WO2021156267, which is incorporated by reference herein). Accordingly, such a procedure (as exemplified for Alanine) is applied for each amino acid encoded by the coding sequence of the RNA to obtain sequences adapted to human codon usage. Human codon usage adapted coding sequences are indicated by the abbreviation “opt3” or “human”.

In some embodiments, the coding RNA may be modified, wherein the G/C content of the at least one coding sequence may be modified compared to the G/C content of the corresponding wild type or reference coding sequence (herein referred to as “G/C modified coding sequence”). In this context, the terms “G/C optimization” or “G/C content modification” relate to a nucleic acid that comprises a modified, for example an increased number of guanosine and/or cytosine nucleotides as compared to the corresponding wild type or reference coding sequence. Such an increased number may be generated by substitution of codons containing adenosine or thymidine nucleotides by codons containing guanosine or cytosine nucleotides. Advantageously, RNA sequences having an increased G/C content are more stable or show a better expression than sequences having an increased A/U. For example, the G/C content of the coding sequence of the RNA is increased by at least 10%, 20%, 30%, for example by at least 40% compared to the G/C content of the coding sequence of the corresponding wild type or reference nucleic acid sequence (herein referred to “opt5” or “gc mod”).

In some embodiments, the coding RNA may be modified, wherein the codon adaptation index (CAI) may be increased or for example maximised in the at least one coding sequence (herein referred to as “CAI maximized coding sequence”). It is suitable that all codons of the wild type or reference nucleic acid sequence that are relatively rare in e.g. a human are exchanged for a respective codon that is frequent in the e.g. a human, wherein the frequent codon encodes the same amino acid as the relatively rare codon. Suitably, the most frequent codons are used for each amino acid of the encoded protein (see Table 2 of published PCT patent application WO2021156267, most frequent human codons are marked with asterisks). Suitably, the RNA comprises at least one coding sequence, wherein the codon adaptation index (CAI) of the at least one coding sequence is at least 0.5, at least 0.8, at least 0.9 or at least 0.95. For example, the codon adaptation index (CAI) of the at least one coding sequence is 1 (CAI=1). For example, in the case of the amino acid Ala, the wild type or reference coding sequence may be adapted in a way that the most frequent human codon “GCC” is always used for said amino acid. Accordingly, such a procedure (as exemplified for Alanine) may be applied for each amino acid encoded by the coding sequence of the nucleic acid to obtain CAI maximized coding sequences (herein referred to as “opt4” or “main”).

In some embodiments, the coding RNA of the first aspect comprises a coding sequence that comprises at least one of the nucleic acid sequences being identical or at least 70%, 80%, 85%, 86%, 87%, 88%, 89%, 90%, 91%, 92%, 93%, 94%, 95%, 96%, 97%, 98%, or 99% identical to the sequences according to any one of SEQ ID NOs: 197-246, 267-316, 523-545, 548-570, 573-595, 598-620, 623-645, 648-670, or a fragment or variant thereof.

In some embodiments, the coding RNA of the first aspect comprises a coding sequence that comprises at least one of the nucleic acid sequences being identical or at least 90%, 91%, 92%, 93%, 94%, 95%, 96%, 97%, 98%, or 99% identical to the sequences according to any one of SEQ ID NOs: 523-545, 548-570, 573-595, 598-620, 623-645, 648-670, or a fragment or variant thereof.

In some embodiments, the at least one coding sequence of the RNA of the disclosure is G/C optimized coding sequence.

In some embodiments, the coding RNA of the first aspect comprises a coding sequence that comprises at least one of the nucleic acid sequences being identical or at least 70%, 80%, 85%, 86%, 87%, 88%, 89%, 90%, 91%, 92%, 93%, 94%, 95%, 96%, 97%, 98%, or 99% identical to the sequences according to any one of SEQ ID NOs: 197-206, 207-216, 237-246, 267-276, 277-286, 307-316, 523-545, 548-570, 648-670, or a fragment or variant thereof.

In some embodiments, the coding RNA of the first aspect comprises a coding sequence that comprises at least one of the nucleic acid sequences being identical or at least 90%, 91%, 92%, 93%, 94%, 95%, 96%, 97%, 98%, or 99% identical to the sequences according to any one of SEQ ID NOs: 523-545, 548-570, 648-670, or a fragment or variant thereof.

In some embodiments, the coding RNA of the first aspect comprises a coding sequence that comprises at least one of the nucleic acid sequences being identical or at least 70%, 80%, 85%, 86%, 87%, 88%, 89%, 90%, 91%, 92%, 93%, 94%, 95%, 96%, 97%, 98%, or 99% identical to the sequences according to any one of SEQ ID NOs: 529, 533, 534, 554, 558, 559, 654, 658, 659, or a fragment or variant of any of these sequences.

In some embodiments, the coding sequence comprises more than one stop codon to allow sufficient termination of translation. In particularly embodiments, the coding sequence comprises two or three stop codon to allow sufficient termination of translation. These more than one stop codons may optionally be positioned in alternative reading frames.

### UTRs:

The RNA of the first aspect comprises at least one untranslated region (UTR).

The term “untranslated region” or “UTR” or “UTR element” will be recognized and understood by the person of ordinary skill in the art, and are e.g. intended to refer to a part of a nucleic acid molecule typically located 5′ or 3′ of a coding sequence. An UTR is not translated into protein. An UTR may be part of the RNA. An UTR may comprise elements for controlling gene expression, also called regulatory elements. Such regulatory elements may be, e.g., ribosomal binding sites, miRNA binding sites, promotor elements etc.

In some embodiments, the coding RNA comprises a protein-coding region (“coding sequence” or “cds”), and a 5′-UTR and/or 3′-UTR. Notably, UTRs may harbour regulatory sequence elements that determine RNA turnover, stability, and localization. Moreover, UTRs may harbour sequence elements that enhance translation. In medical applications, translation of the RNA into at least one peptide or protein is of paramount importance to therapeutic efficacy. Certain combinations of 3′-UTRs and/or 5′-UTRs may enhance the expression of operably linked coding sequences encoding peptides or proteins as defined herein. RNA molecules harbouring said UTR combinations advantageously enable rapid and transient expression of antigenic peptides or proteins after administration to a subject, for example after intramuscular administration. Accordingly, the RNA of the disclosure comprising certain combinations of 3′-UTRs and/or 5′-UTRs is particularly suitable for administration as a vaccine, in particular, suitable for administration into the muscle, the dermis, or the epidermis of a subject.

Suitably, the coding RNA comprises at least one 5′-UTR and/or at least one 3′-UTR. Said heterologous 5′-UTRs or 3′-UTRs may be derived from naturally occurring genes or may be synthetically engineered. In some embodiments, the RNA comprises at least one coding sequence as defined herein operably linked to at least one (heterologous) 3′-UTR and/or at least one (heterologous) 5′-UTR.

In some embodiments, the coding RNA of the disclosure comprises at least one 3′-UTR.

The term “3′-untranslated region” or “3′-UTR” will be recognized and understood by the person of ordinary skill in the art, and are e.g. intended to refer to a part of an RNA molecule located 3′ (i.e. downstream) of a coding sequence and which is not translated into protein. A 3′-UTR may be part of a nucleic acid located between a coding sequence and an (optional) terminal poly(A) sequence. A 3′-UTR may comprise elements for controlling gene expression, also called regulatory elements. Such regulatory elements may be, e.g., ribosomal binding sites, miRNA binding sites etc.

Optionally, the coding RNA comprises at least one 3′-UTR, which may be derivable from a gene that relates to an RNA with enhanced half-life (i.e. that provides a stable RNA).

In some embodiments, the 3′-UTR comprises one or more of a polyadenylation signal, a binding site for proteins that affect a nucleic acid stability of location in a cell, or one or more miRNA or binding sites for miRNAs.

MicroRNAs (or miRNA) are about 19-25 nucleotide long noncoding RNAs that bind to the 3′-UTR of RNA molecules and down-regulate gene expression either by reducing RNA stability or by inhibiting translation. E.g., microRNAs are known to regulate RNA, and thereby protein expression, e.g. in liver (miR-122), heart (miR-Id, miR-149), endothelial cells (miR-17-92, miR-126), adipose tissue (let-7, miR-30c), kidney (miR-192, miR-194, miR-204), myeloid cells (miR-142-3p, miR-142-5p, miR-16, miR-21, miR-223, miR-24, miR-27), muscle (miR-133, miR-206, miR-208), and lung epithelial cells (let-7, miR-133, miR-126). The RNA may comprise one or more microRNA target sequences, microRNA sequences, or microRNA seeds. Such sequences may correspond to any known microRNA, e.g. to those taught in US20050261218 and US20050059005, which are incorporated by reference herein.

Accordingly, miRNA, or binding sites for miRNAs as defined above may be removed from the 3′-UTR or may be introduced into the 3′-UTR in order to tailor the expression of the RNA to desired cell types or tissues (e.g. muscle cells).

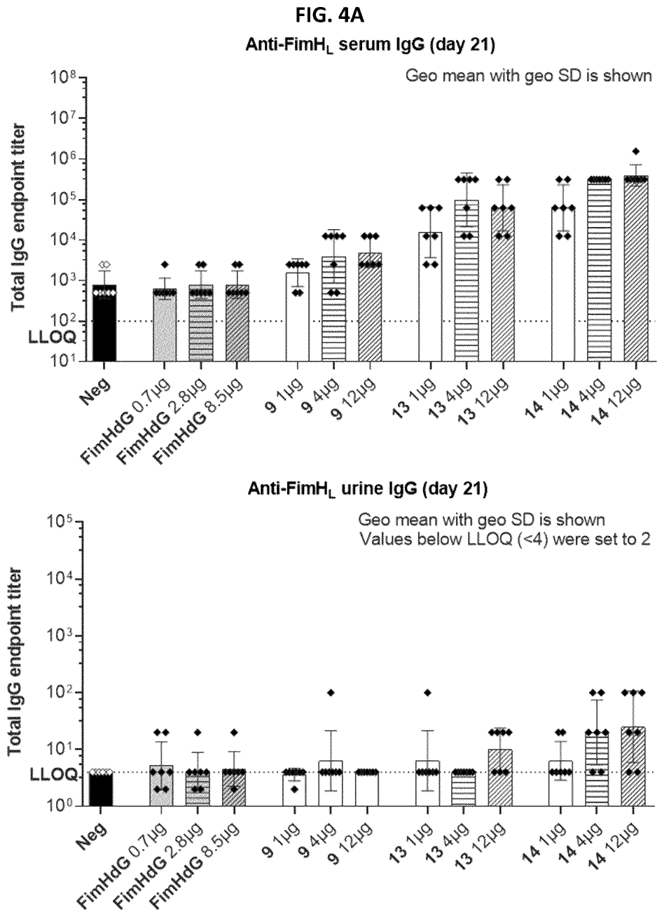

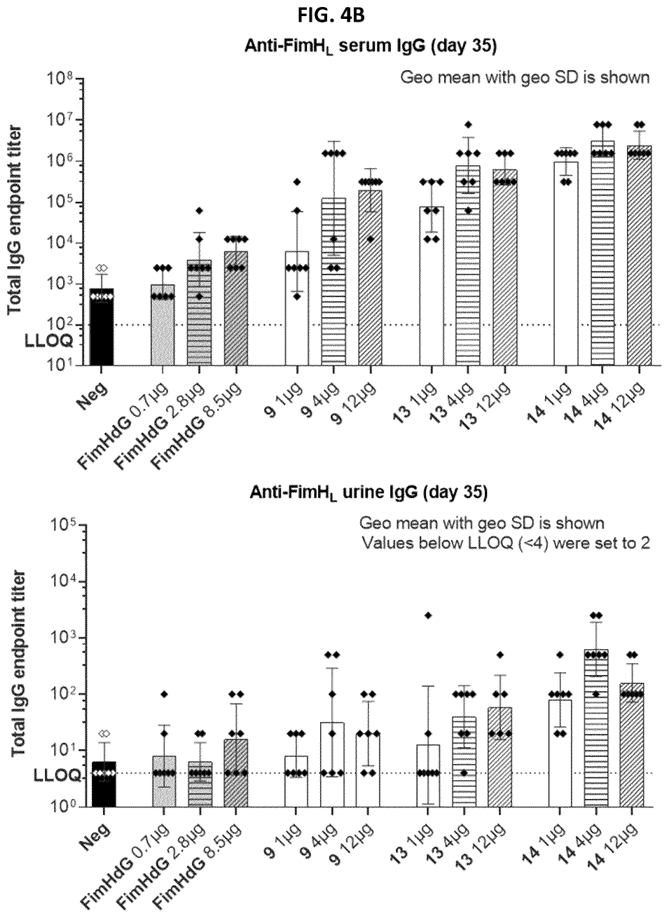

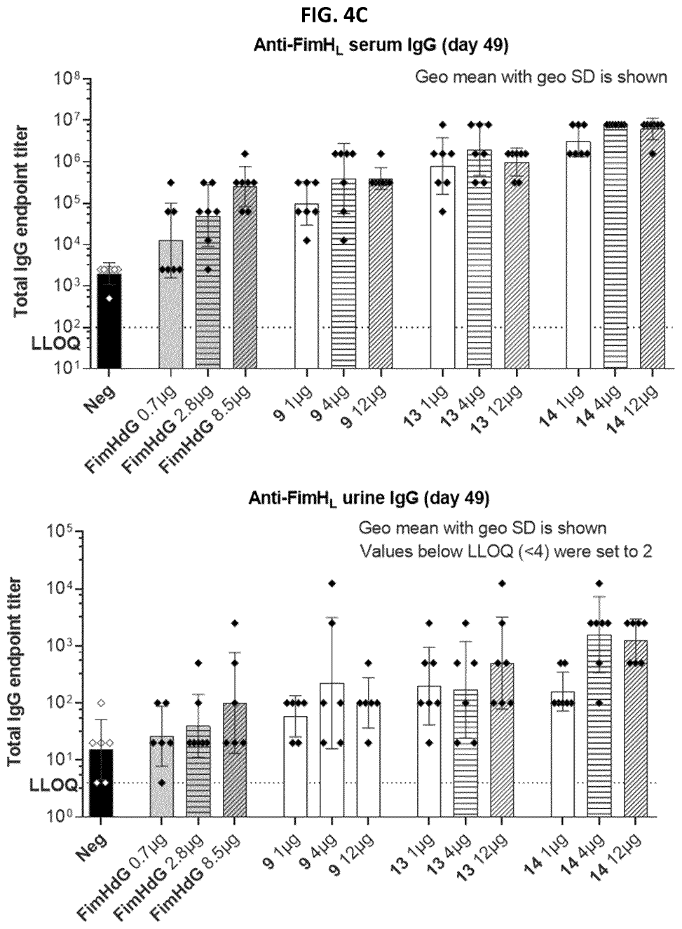

In some embodiments, the coding RNA comprises at least one 3′-UTR, wherein the at least one 3′-UTR comprises a nucleic acid sequence derived or selected from a 3′-UTR of a gene selected from PSMB3, alpha-globin, ALB7, CASP1, COX6B1, FIG. 4, GNAS, NDUFA1 and RPS9, or from a homolog, a fragment, or variant of any one of these genes.

In some embodiments, the at least one 3′-UTR derived or selected from PSMB3, alpha-globin, ALB7, CASP1, COX6B1, FIG. 4, GNAS, NDUFA1 or RPS9 comprises or consist of a nucleic acid sequence being identical or at least 70%, 80%, 85%, 86%, 87%, 88%, 89%, 90%, 91%, 92%, 93%, 94%, 95%, 96%, 97%, 98%, or 99% identical to any one of SEQ ID NOs: 67-90, 109-120, or a fragment or a variant of any of these.

In one embodiment, the coding RNA comprises a 3′-UTR derived or selected from a PSMB3 gene.

In one embodiment, the 3′-UTR derived or selected from PSMB3 comprises or consist of a nucleic acid sequence being identical or at least 70%, 80%, 85%, 86%, 87%, 88%, 89%, 90%, 91%, 92%, 93%, 94%, 95%, 96%, 97%, 98%, or 99% identical to any one of SEQ ID NOs: 67, 68, 109-120, or a fragment or a variant thereof.

In other embodiments, the coding RNA comprises a 3′-UTR which comprises or consists of a nucleic acid sequence being identical or at least 70%, 80%, 85%, 86%, 87%, 88%, 89%, 90%, 91%, 92%, 93%, 94%, 95%, 96%, 97%, 98%, or 99% identical to SEQ ID NOs: 91-108 or a fragment or a variant thereof.

In other embodiments, the coding RNA comprises a 3′-UTR as described in WO2016107877, the disclosure of WO2016107877 relating to 3′-UTR sequences herewith incorporated by reference. Suitable 3′-UTRs are SEQ ID NOs: 1-24 and SEQ ID NOs: 49-318 of WO2016107877, or fragments or variants of these sequences. In other embodiments, the RNA comprises a 3′-UTR as described in WO2017036580, the disclosure of WO2017036580 relating to 3′-UTR sequences herewith incorporated by reference. Suitable 3′-UTRs are SEQ ID NOs: 152-204 of WO2017036580, or fragments or variants of these sequences. In other embodiments, the RNA comprises a 3′-UTR as described in WO2016022914, the disclosure of WO2016022914 relating to 3′-UTR sequences herewith incorporated by reference. Particularly suitable 3′-UTRs are nucleic acid sequences according to SEQ ID NOs: 20-36 of WO2016022914, or fragments or variants of these sequences.

In some embodiments, the coding RNA of the disclosure comprises at least one 5′-UTR.

The terms “5′-untranslated region” or “5′-UTR” will be recognized and understood by the person of ordinary skill in the art, and are e.g. intended to refer to a part of an RNA located 5′ (i.e. “upstream”) of a coding sequence and which is not translated into protein. A 5′-UTR may be part of a nucleic acid located 5′ of the coding sequence. Typically, a 5′-UTR starts with the transcriptional start site and ends before the start codon of the coding sequence. A 5′-UTR may comprise elements for controlling gene expression, also called regulatory elements. Such regulatory elements may be, e.g., ribosomal binding sites, miRNA binding sites etc. The 5′-UTR may be modified, e.g. by enzymatic or co-transcriptional addition of a 5′-cap structure (e.g. for mRNA as defined below).

Optionally, the coding RNA comprises at least one 5′-UTR, which may be derivable from a gene that relates to an RNA with enhanced half-life (i.e. that provides a stable RNA).

In some embodiments, the 5′-UTR comprises one or more of a binding site for proteins that affect an RNA stability or RNA location in a cell, or one or more miRNA or binding sites for miRNAs (as defined above).

Accordingly, miRNA or binding sites for miRNAs as defined above may be removed from the 5′-UTR or introduced into the 5′-UTR in order to tailor the expression of the nucleic acid to desired cell types or tissues (e.g. muscle cells).

In some embodiments, the coding RNA comprises at least one 5′-UTR, wherein the at least one 5′-UTR comprises a nucleic acid sequence derived or selected from a 5′-UTR of gene selected from HSD17B4, RPL32, ASAH1, ATP5A1, MP68, NDUFA4, NOSIP, RPL31, SLC7A3, TUBB4B, and UBQLN2, or from a homolog, a fragment or variant of any one of these genes.

In some embodiments, the at least one 5′-UTR derived or selected from HSD17B4, RPL32, ASAH1, ATP5A1, MP68, NDUFA4, NOSIP, RPL31, SLC7A3, TUBB4B, and UBQLN2 comprises or consist of a nucleic acid sequence being identical or at least 70%, 80%, 85%, 86%, 87%, 88%, 89%, 90%, 91%, 92%, 93%, 94%, 95%, 96%, 97%, 98%, or 99% identical to any one of SEQ ID NOs: 1-32, 65, 66, or a fragment or a variant of any of these.

In one embodiment, the coding RNA comprises a 5′-UTR derived or selected from a HSD17B4 gene.

In one embodiment, the 5′-UTR derived or selected from HSD17B4 comprises or consists of a nucleic acid sequence being identical or at least 70%, 80%, 85%, 86%, 87%, 88%, 89%, 90%, 91%, 92%, 93%, 94%, 95%, 96%, 97%, 98%, or 99% identical to any one of SEQ ID NOs: 1, 2, 65, 66, or a fragment or a variant thereof.

In other embodiments, the coding RNA comprises a 5′-UTR which comprises or consists of a nucleic acid sequence being identical or at least 70%, 80%, 85%, 86%, 87%, 88%, 89%, 90%, 91%, 92%, 93%, 94%, 95%, 96%, 97%, 98%, or 99% identical to SEQ ID NOs: 33-64 or a fragment or a variant thereof.

In other embodiments, the coding RNA comprises a 5′-UTR as described in WO2013143700, the disclosure of WO2013143700 relating to 5′-UTR sequences herewith incorporated by reference. Particularly suitable 5′-UTRs are nucleic acid sequences derived from SEQ ID NOs: 1-1363, SEQ ID NO: 1395, SEQ ID NO: 1421 and SEQ ID NO: 1422 of WO2013143700, or fragments or variants of these sequences. In other embodiments, the coding RNA comprises a 5′-UTR as described in WO2016107877, the disclosure of WO2016107877 relating to 5′-UTR sequences herewith incorporated by reference. Particularly suitable 5′-UTRs are nucleic acid sequences according to SEQ ID NOs: 25-30 and SEQ ID NOs: 319-382 of WO2016107877, or fragments or variants of these sequences. In other embodiments, the RNA comprises a 5′-UTR as described in WO2017036580, the disclosure of WO2017036580 relating to 5′-UTR sequences herewith incorporated by reference. Particularly suitable 5′-UTRs are nucleic acid sequences according to SEQ ID NOs: 1-151 of WO2017036580, or fragments or variants of these sequences. In other embodiments, the RNA comprises a 5′-UTR as described in WO2016022914, the disclosure of WO2016022914 relating to 5′-UTR sequences herewith incorporated by reference. Suitable 5′-UTRs are nucleic acid sequences according to SEQ ID NOs: 3-19 of WO2016022914, whose disclosure is incorporated by reference herein, or fragments or variants of these sequences.

In some embodiments, the coding RNA of the disclosure comprises a coding sequence as specified herein encoding an antigenic polypeptide which is selected or derived from Escherichia coli FimH, and a 3′-UTR and/or a 5′-UTR selected from the following 5′-UTR/3′-UTR combinations (also referred to “UTR designs”): a-1 (HSD17B4/PSMB3), a-2 (NDUFA4/PSMB3), a-3 (SLC7A3/PSMB3), a-4 (NOSIP/PSMB3), a-5 (MP68/PSMB3), b-1 (UBQLN2/RPS9), b-2 (ASAH1/RPS9), b-3 (HSD17B4/RPS9), b-4 (HSD17B4/CASP1), b-5 (NOSIP/COX6B1), c-1 (NDUFA4/RPS9), c-2 (NOSIP/NDUFA1), c-3 (NDUFA4/COX6B1), c-4 (NDUFA4/NDUFA1), c-5 (ATP5A1/PSMB3), d-1 (Rpl31/PSMB3), d-2 (ATP5A1/CASP1), d-3 (SLC7A3/GNAS), d-4 (HSD17B4/NDUFA1), d-5 (Slc7a3/Ndufa1), e-1 (TUBB4B/RPS9), e-2 (RPL31/RPS9), e-3 (MP68/RPS9), e-4 (NOSIP/RPS9), e-5 (ATP5A1/RPS9), e-6 (ATP5A1/COX6B1), f-1 (ATP5A1/GNAS), f-2 (ATP5A1/NDUFA1), f-3 (HSD17B4/COX6B1), f-4 (HSD17B4/GNAS), f-5 (MP68/COX6B1), g-1 (MP68/NDUFA1), g-2 (NDUFA4/CASP1), g-3 (NDUFA4/GNAS), g-4 (NOSIP/CASP1), g-5 (RPL31/CASP1), h-1 (RPL31/COX6B1), h-2 (RPL31/GNAS), h-3 (RPL31/NDUFA1), h-4 (Slc7a3/CASP1), h-5 (SLC7A3/COX6B1), i-1 (SLC7A3/RPS9), i-2 (RPL32/ALB7), i-2 (RPL32/ALB7), or i-3 (alpha-globin gene).

In some embodiments, the coding RNA comprises a coding sequence as defined herein encoding an antigenic polypeptide which is selected or derived from Escherichia coli FimH, and a HSD17B4 5′-UTR and a PSMB3 3′-UTR (HSD17B4/PSMB3 (a-1)). It has been shown by the inventors that this embodiment is particularly beneficial for induction of an immune response against Escherichia coli FimH.

In various embodiments, the coding RNA of the disclosure is monocistronic, bicistronic, or multicistronic.

In some embodiments, the coding RNA of the disclosure is monocistronic.

The term “monocistronic” will be recognized and understood by the person of ordinary skill in the art, and is e.g. intended to refer to an RNA that comprises only one coding sequence. The terms “bicistronic”, or “multicistronic” as used herein are e.g. intended to refer to an RNA that comprises two (bicistronic) or more (multicistronic) coding sequences.

In some embodiments, the A/U (A/T) content in the environment of the ribosome binding site of the RNA is increased compared to the A/U (A/T) content in the environment of the ribosome binding site of its respective wild type or reference nucleic acid. This modification increases the efficiency of ribosome binding to the RNA, which is in turn beneficial for an efficient translation of the RNA into antigenic peptides or proteins.

Accordingly, in one embodiment, the coding RNA comprises a ribosome binding site, also referred to as “Kozak sequence” identical to or at least 80%, 85%, 90%, 95% identical to any one of SEQ ID NOs: 128-135, or fragments or variants of any of these.

### Poly(N)Sequences, Histone Stem Loops:

In some embodiments, the coding RNA comprises at least one poly(N) sequence, e.g. at least one poly(A) sequence, at least one poly(U) sequence, at least one poly(C) sequence, or combinations thereof.

In some embodiments, the coding RNA of the disclosure comprises at least one poly(A) sequence.

The terms “poly(A) sequence”, “poly(A) tail” or “3′-poly(A) tail” as used herein will be recognized and understood by the person of ordinary skill in the art, and are e.g. intended to be a sequence of adenosine nucleotides, typically located at the 3′-end of a linear RNA of up to about 1000 adenosine nucleotides. For example, said poly(A) sequence is essentially homopolymeric, e.g. a poly(A) sequence of e.g. 100 adenosine nucleotides has essentially the length of 100 nucleotides. In other embodiments, the poly(A) sequence is interrupted by at least one nucleotide different from an adenosine nucleotide, e.g. a poly(A) sequence of e.g. 100 adenosine nucleotides may have a length of more than 100 nucleotides (comprising 100 adenosine nucleotides and in addition said at least one nucleotide—or a stretch of nucleotides—different from an adenosine nucleotide).

In some embodiments, the at least one poly(A) sequence may comprise about 40 to about 500 adenosine nucleotides, about 40 to about 200 adenosine nucleotides, about 40 to about 150 adenosine nucleotides, for example about 60 to about 150 adenosine nucleotides.

In some embodiment, the at least one poly(A) sequence may comprise about 40 to about 500 consecutive adenosine nucleotides, about 40 to about 200 consecutive adenosine nucleotides, about 40 to about 150 consecutive adenosine nucleotides, for example about 60 to about 150 consecutive adenosine nucleotides.

Suitably, the length of the poly(A) sequence may be at least about or even more than about 10, 50, 64, 75, 100, 200, 300, 400, or 500 adenosine nucleotides, for example consecutive adenosine nucleotides.

In some embodiments, the at least one poly(A) sequence comprises about 100 adenosine nucleotides (A100), for example about 100 consecutive adenosine nucleotides.

In further embodiments, the RNA comprises at least one poly(A) sequence comprising about 100 adenosine nucleotides, wherein the poly(A) sequence is interrupted by non-adenosine nucleotides, for example by about 10 non-adenosine nucleotides (A30-N10-A70).

Suitably, the poly(A) sequence as defined herein may be located directly at the 3′ terminus of the RNA. In some embodiments, the 3′-terminal nucleotide (that is the last 3′-terminal nucleotide in the polynucleotide chain) is the 3′-terminal A nucleotide of the at least one poly(A) sequence. The term “directly located at the 3′ terminus” has to be understood as being located exactly at the 3′ terminus—in other words, the 3′ terminus of the RNA consists of a poly(A) sequence terminating with an A.

Ending on an adenosine nucleotide may decrease the induction of interferons, e.g. IFNalpha, by the RNA of the disclosure if for example administered as a vaccine. This is particularly important as the induction of interferons, e.g. IFNalpha, is thought to be one main factor for induction of fever in vaccinated subjects.

Accordingly, in some embodiments, the coding RNA of the disclosure comprises a poly(A) sequence of about 100 consecutive adenosine nucleotides, wherein said poly(A) sequence is located directly at the 3′ terminus of the RNA, optionally wherein the 3′ terminal nucleotide is an adenosine.

In some embodiments, the poly(A) sequence of the RNA is obtained from a DNA template during RNA in vitro transcription. In other embodiments, the poly(A) sequence is obtained in vitro by common methods of chemical synthesis without being necessarily transcribed from a DNA template. In other embodiments, poly(A) sequences are generated by enzymatic polyadenylation of the RNA (after RNA in vitro transcription) using e.g. immobilized poly(A)polymerases according to methods and means as described in WO2016174271, which is incorporated by reference herein.

In some embodiments, the coding RNA comprises at least one poly(A) sequence obtained by enzymatic polyadenylation, wherein the majority of RNA molecules comprise about 100 (+/−20) to about 500 (+/−100) adenosine nucleotides, for example about 100 (+/−20) to about 200 (+/−40) adenosine nucleotides.

In some embodiments, the coding RNA comprises at least one poly(A) sequence derived from a template DNA and additionally at least one poly(A) sequence generated by enzymatic polyadenylation, e.g. as described in published PCT patent application WO2016091391, which is incorporated by reference herein.

In some embodiments, the coding RNA comprises at least one polyadenylation signal.

In some embodiments, the coding RNA comprises at least one poly(C) sequence. A poly(C) sequence in the context of the disclosure may be located in an UTR region, for example in the 3′-UTR.

The term “poly(C) sequence” as used herein is intended to be a sequence of cytosine nucleotides of up to about 200 cytosine nucleotides. In some embodiments, the poly(C) sequence comprises about 10 to about 200 cytosine nucleotides, about 10 to about 100 cytosine nucleotides, about 20 to about 70 cytosine nucleotides, about 20 to about 60 cytosine nucleotides, or about 10 to about 40 cytosine nucleotides. In one embodiment, the poly(C) sequence comprises about 30 cytosine nucleotides.

In some embodiment, the coding RNA of the disclosure comprises at least one poly(C) sequence and/or at least one miRNA binding site and/or histone stem-loop sequence.

In some embodiments, the coding RNA of the disclosure comprises at least one histone stem-loop (hSL) or histone stem-loop structure. A hSL in the context of the disclosure may be located in an UTR region, for example in the 3′-UTR.

The term “histone stem-loop” (hSL) is intended to refer to nucleic acid sequences that forms a stem-loop secondary structure predominantly found in histone mRNAs.

Histone stem-loop sequences/structures may suitably be selected from hSL sequences as disclosed in WO2012019780, the disclosure relating to histone stem-loop sequences/histone stem-loop structures incorporated herewith by reference. A hSL sequence that may be used within the present disclosure may be derived from formulae (I) or (II) of WO2012019780. According to one embodiment, the RNA comprises at least one hSL sequence derived from at least one of the specific formulae (Ia) or (IIa) of WO2012019780.

In some embodiments, the coding RNA comprises at least one hSL, wherein said hSL comprises or consists of an RNA sequence identical or at least 70%, 80%, 90%, 91%, 92%, 93%, 94%, 95%, 96%, 97%, 98%, or 99% identical to any one of SEQ ID NOs: 136, 137, or fragments or of those.

In alternative embodiments, the coding RNA does not comprise a histone stem-loop as defined herein.

In some embodiments, the coding RNA comprises a 3′-terminal sequence element. The 3′-terminal sequence element represents the 3′ terminus of the RNA. A 3′-terminal sequence element may comprise at least one poly(N) sequence as defined herein and, optionally, at least one hSL as defined herein.

In some embodiments, the coding RNA comprises at least one 3′-terminal sequence element comprising or consisting of an RNA sequence being identical or at least 70%, 80%, 90%, 91%, 92%, 93%, 94%, 95%, 96%, 97%, 98%, or 99% identical to any one of SEQ ID NOs: 138-172, or a fragment or variant of these sequences.

In some embodiments, the coding RNA comprises a 3′-terminal sequence element comprising a hSL as defined herein followed by a poly(A) sequence comprising about 100 consecutive adenosines.

In some embodiments, the coding RNA comprises a 3′-terminal sequence element comprising or consisting of an RNA sequence being identical or at least 70%, 80%, 90%, 91%, 92%, 93%, 94%, 95%, 96%, 97%, 98%, or 99% identical to SEQ ID NOs: 144, or a fragment or variant thereof.

In some embodiments, the coding RNA comprises a 5′-terminal sequence element comprising or consisting of an RNA sequence being identical or at least 70%, 80%, 90%, 91%, 92%, 93%, 94%, 95%, 96%, 97%, 98%, or 99% identical to any one of SEQ ID NOs: 121-127, or a fragment or variant of these sequences.

In some embodiments, the coding RNA comprises a 5′-terminal sequence element comprising or consisting of an RNA sequence being identical or at least 70%, 80%, 90%, 91%, 92%, 93%, 94%, 95%, 96%, 97%, 98%, or 99% identical to SEQ ID NOs: 122, or a fragment or variant thereof.

Such a 5′-terminal sequence element may comprise e.g. a binding site for T7 RNA polymerase. Further, the first nucleotide of said 5′-terminal start sequence may for example comprise a 2′O methylation, e.g. 2′O methylated guanosine or a 2′O methylated adenosine.

### Cap Structures:

Suitably, the coding RNA comprises a 5′-cap structure, which suitably stabilizes the RNA and/or enhances expression of the encoded antigen and/or reduces the stimulation of the innate immune system (after administration to a subject, e.g. a human subject).

Accordingly, in some embodiments, the coding RNA comprises a 5′-cap structure, such as m7G, cap0, cap1, cap2, a modified cap0 or a modified cap1 structure.

The term “5′-cap structure” as used herein will be recognized and understood by the person of ordinary skill in the art, and is e.g. intended to refer to a 5′ modified nucleotide, particularly a guanine nucleotide, positioned at the 5′-end of an RNA, e.g. an mRNA. For example, the 5′-cap structure is connected via a 5′-5′-triphosphate linkage to the RNA. 5′-cap structures which may be suitable in the context of the present disclosure are cap0 (methylation of the first nucleobase, e.g. m7GpppN), cap1 (additional methylation of the ribose of the adjacent nucleotide of m7GpppN), cap2 (additional methylation of the ribose of the 2nd nucleotide downstream of the m7GpppN), cap3 (additional methylation of the ribose of the 3rd nucleotide downstream of the m7GpppN), cap4 (additional methylation of the ribose of the 4th nucleotide downstream of the m7GpppN), ARCA (anti-reverse cap analogue), modified ARCA (e.g. phosphothioate modified ARCA), inosine, N1-methyl-guanosine, 2′-fluoro-guanosine, 7-deaza-guanosine, 8-oxo-guanosine, 2-amino-guanosine, LNA-guanosine, and 2-azido-guanosine.

A 5′-cap (cap0 or cap1) structure may be formed in chemical RNA synthesis or in RNA in vitro transcription (co-transcriptional capping) using cap analogues.

The term “cap analogue” as used herein will be recognized and understood by the person of ordinary skill in the art, and is e.g. intended to refer to a non-polymerizable di-nucleotide or tri-nucleotide that has cap functionality in that it facilitates translation or localization, and/or prevents degradation of an RNA molecule when incorporated at the 5′-end of the nucleic acid molecule. Non-polymerizable means that the cap analogue will be incorporated only at the 5′-terminus because it does not have a 5′ triphosphate and therefore cannot be extended in the 3′-direction by a template-dependent polymerase, particularly, by template-dependent RNA polymerase. Examples of cap analogues include, but are not limited to, a chemical structure selected from the group consisting of m7GpppG, m7GpppA, m7GpppC; unmethylated cap analogues (e.g. GpppG); dimethylated cap analogue (e.g. m2,7GpppG), trimethylated cap analogue (e.g. m2,2,7GpppG), dimethylated symmetrical cap analogues (e.g. m7Gpppm7G), or anti reverse cap analogues (e.g. ARCA; m7,2′OmeGpppG; m7,2′dGpppG; m7,3′OmeGpppG; m7,3′dGpppG and their tetraphosphate derivatives). Further suitable cap analogues are described in WO2008016473, WO2008157688, WO2009149253, WO2011015347, WO2013059475, WO2017066793, WO2017066781, WO2017066791, WO2017066789, WO2017053297, WO2017066782, WO2018075827 and WO2017066797, the disclosures referring to cap analogues herewith incorporated by reference.

In embodiments, a cap1 structure is generated using tri-nucleotide cap analogue as disclosed in WO2017053297, WO2017066793, WO2017066781, WO2017066791, WO2017066789, WO2017066782, WO2018075827 and WO2017066797. For example, cap structures derivable from the structure disclosed in claim 1-5 of WO2017053297 may be suitably used to co-transcriptionally generate a cap1 structure. Further, any cap structures derivable from the structure defined in claim 1 or claim 21 of WO2018075827 may be suitably used to generate a cap1 structure. These disclosures are herewith incorporated by reference.

In some embodiments, the 5′-cap structure may suitably be added co-transcriptionally using tri-nucleotide cap analogue as defined herein, in particular in an RNA in vitro transcription reaction as defined herein.

In some embodiments, the coding RNA of the disclosure, in particular the mRNA, comprises a cap1 structure.

In some embodiments, the cap1 structure of the RNA is formed via co-transcriptional capping using tri-nucleotide cap analogues m7G(5′)ppp(5′)(2′OMeA)pG or m7G(5′)ppp(5′)(2′OMeG)pG. A particularly suitable cap1 analogue in that context is m7G(5′)ppp(5′)(2′OMeA)pG.

In other embodiments, the cap1 structure of the RNA is formed using co-transcriptional capping using tri-nucleotide cap analogue 3′OMe-m7G(5′)ppp(5′)(2′OMeA)pG.

In alternative embodiments, the 5′-cap structure is formed via enzymatic capping using capping enzymes (e.g. vaccinia virus capping enzymes and/or cap-dependent 2′-O methyltransferases) to generate cap0 or cap1 or cap2 structures. In that context, the 5′-cap structure (cap0 or cap1) may be added using immobilized capping enzymes and/or cap-dependent 2′-O methyltransferases using methods and means disclosed in published PCT patent application WO2016193226, which is incorporated by reference herein.

In some embodiments, about 70%, 75%, 80%, 85%, 90%, 95% of the RNA (species) comprises a cap structure, for example a cap1 structure as determined by a capping assay.

For determining the presence or absence of a cap structure, a capping assay as described in published PCT application WO2015101416, in particular, as described in claims 27 to 46 of published PCT application WO2015101416 can be used. Other capping assays that may be used to determine the presence or absence of a cap structure of an RNA are described in published PCT application WO2020127959. These disclosures are herewith incorporated by reference.

### Modified Nucleotides:

According to various embodiments, the coding RNA of the disclosure is a modified RNA, wherein the modification refers to chemical modifications comprising backbone modifications as well as sugar modifications or base modifications.

A modified RNA may comprise nucleotide analogues/modifications, e.g. backbone modifications, sugar modifications or base modifications. A backbone modification in the context of the disclosure is a modification in which phosphates of the backbone of the nucleotides of the RNA are chemically modified. A sugar modification in the context of the disclosure is a chemical modification of the sugar of the nucleotides of the RNA. Furthermore, a base modification in the context of the disclosure is a chemical modification of the base moiety of the nucleotides of the RNA. In this context, nucleotide analogues or modifications are for example selected from nucleotide analogues which are applicable for transcription and/or translation.

Accordingly, in some embodiments, the coding RNA of the disclosure comprises at least one modified nucleotide.

In some embodiments, the at least one modified nucleotide is selected from pseudouridine, N1-methylpseudouridine, N1-ethylpseudouridine, 2-thiouridine, 4-thiouridine, 5-methylcytosine, 5-methyluridine, 2-thio-1-methyl-1-deaza-pseudouridine, 2-thio-1-methyl-pseudouridine, 2-thio-5-aza-uridine, 2-thio-dihydropseudouridine, 2-thio-dihydrouridine, 2-thio-pseudouridine, 4-methoxy-2-thio-pseudouridine, 4-methoxy-pseudouridine, 4-thio-1-methyl-pseudouridine, 4-thio-pseudouridine, 5-aza-uridine, dihydropseudouridine, 5-methoxyuridine and 2′-O-methyl uridine.

Suitable in that context are pseudouridine (ψ) and N1-methylpseudouridine (m1ψ). N1-methylpseudouridine (m1ψ) is particularly suitable.

In some embodiments, essentially all, e.g. essentially 100% of the uracil in the coding sequence (or the full RNA sequence) have a chemical modification, for example a chemical modification in the 5-position of the uracil.

Incorporating modified nucleotides such as e.g. pseudouridine (ψ) or N1-methylpseudouridine (m1ψ) into the coding sequence (or the full RNA sequence) may be advantageous as unwanted innate immune responses (upon administration of the RNA) may be adjusted or reduced (if required).

In alternative embodiments, the coding RNA of the disclosure does not comprise modified nucleotides such as N1-methylpseudouridine (m1ψ) substituted positions or pseudouridine (ψ) substituted positions.

In some embodiments in that context, the coding RNA of the disclosure comprises a coding sequence that consists only of G, C, A and U nucleotides and therefore does not comprise modified nucleotides.

### Further RNA Features:

In the context of the disclosure, the coding RNA provides a coding sequence encoding an antigenic polypeptide which is selected or derived from Escherichia coli FimH as defined herein that is translated into a (functional) antigen after administration (e.g. after administration to a subject, e.g. a human subject).

In the context of the disclosure, a coding RNA can be any type of RNA that comprises a coding sequence encoding an antigenic polypeptide which is selected or derived from Escherichia coli FimH. For example, the coding RNA can be any type of single stranded coding RNA, double stranded coding RNA, linear coding RNA, or circular coding RNA, or any combination thereof.

Optionally, the coding RNA comprises about 50 to about 20000 nucleotides, or about 500 to about 10000 nucleotides, or about 1000 to about 10000 nucleotides, or for example about 1000 to about 5000 nucleotides, or for example about 2000 to about 5000 nucleotides.

In some embodiments, the coding RNA is selected from an mRNA, a coding self-replicating RNA, a coding circular RNA, a coding viral RNA, or a coding replicon RNA.

In embodiments, the coding RNA is a circular RNA. As used herein, “circular RNA” or “circRNAs” have to be understood as an RNA construct that is connected to form a circle and therefore does not comprise a 3′ or 5′ terminus. In some embodiments, said circRNA comprises a coding sequence encoding an antigenic polypeptide which is selected or derived from Escherichia coli FimH as defined herein.

In some embodiments, the coding RNA is an mRNA.

Suitable features that the mRNA of the disclosure optionally comprises are for example a 5′-cap structure as defined herein, a 5′-UTR as defined herein, a 3′-UTR as defined herein, hSL as defined herein, Poly(A)sequence as defined herein, and optional chemical modifications as defined herein.

In some embodiments, the coding RNA is an in vitro transcribed RNA (e.g. an in vitro transcribed mRNA).

In some embodiments, the nucleotide mixture for RNA in vitro transcription comprises modified nucleotides as defined herein. In that context, suitable modified nucleotides may be selected from pseudouridine (ψ) or N1-methylpseudouridine (m1ψ). Suitably, uracil nucleotides in the nucleotide mixture are replaced (either partially or completely) by pseudouridine (ψ) and/or N1-methylpseudouridine (m1ψ) to obtain a modified RNA (e.g. a modified mRNA).

In some embodiments, the nucleotide mixture used in RNA in vitro transcription does not comprise modified nucleotides as defined herein. In some embodiments, the nucleotide mixture used for RNA in vitro transcription does only comprise guanosine (G), cytidine (C), adenosine (A) and uridine (U) nucleotides, and, optionally, a cap analogue as defined herein to obtain a non-modified RNA (e.g. a non-modified mRNA).

In some embodiments, the nucleotide mixture (i.e. the fraction of each nucleotide in the mixture) used for RNA in vitro transcription reactions is optimized for the given RNA sequence, for example as described WO2015188933, which is incorporated by reference herein.

In one embodiment, the coding RNA is lyophilized (e.g. according to WO2016165831 or WO2011069586) to yield a temperature stable dried RNA. The RNA may also be dried using spray-drying or spray-freeze drying (e.g. according to WO2016184575 or WO2016184576) to yield a temperature stable RNA (powder). These disclosures are incorporated by reference herein.

In the context of the disclosure (e.g. for RNA-based vaccines), it may be required to provide GMP-grade RNA. GMP-grade RNA is produced using a manufacturing process approved by regulatory authorities. In some embodiments, RNA production is performed under current good manufacturing practice (GMP), implementing various quality control steps on DNA and RNA level, for example quality control steps selected from methods described in WO2016180430. In some embodiments, the RNA of the disclosure is a GMP-grade RNA, for example a GMP-grade mRNA.

In some embodiments, the coding RNA of the disclosure is a purified RNA, optionally a purified mRNA.

The term “purified RNA” or “purified mRNA” as used herein has to be understood as RNA which has a higher purity after certain purification steps (e.g. HPLC, TFF, Oligo d(T) purification, precipitation steps) than the starting material (e.g. in vitro transcribed RNA). Typical impurities that are essentially not present in purified RNA comprise peptides or proteins (e.g. enzymes derived from DNA dependent RNA in vitro transcription, e.g. RNA polymerases, RNases, pyrophosphatase, restriction endonuclease, DNase), spermidine, BSA, abortive RNA sequences, RNA fragments (short double stranded RNA (dsRNA)), free nucleotides (modified nucleotides, conventional NTPs, cap analogue), template DNA fragments, buffer components (HEPES, TRIS, MgCl2) etc. Other potential impurities that may be derived from e.g. fermentation procedures comprise bacterial impurities (bioburden, bacterial DNA) or impurities derived from purification procedures (organic solvents etc.). Accordingly, it is desirable in this regard for the “degree of RNA purity” to be as close as possible to 100%. Accordingly, “purified RNA” as used herein has a degree of purity of more than 75%, 80%, 85%, very particularly 90%, 91%, 92%, 93%, 94%, 95%, 96%, 97%, 98% and most favourably 99% or more. The degree of purity is e.g. determined by an analytical HPLC, wherein the percentages provided above correspond to the ratio between the area of the peak for the target RNA and the total area of all peaks including the peaks representing the by-products. Alternatively, the degree of purity is e.g. determined by an analytical agarose gel electrophoresis or capillary gel electrophoresis.

Suitably, purification of the coding RNA of the disclosure may be performed by means of (RP)-HPLC, AEX, size exclusion chromatography, hydroxyapatite chromatography, TFF, filtration, precipitation, core-bead flow through chromatography, oligo(dT) purification, and/or cellulose-based purification.

Optionally, the RNA has been purified using RP-HPLC (for example as described in WO2008077592) and/or tangential flow filtration (for example as described in WO2016193206) and/or oligo d(T) purification (for example as described in WO2016180430) to e.g. to remove dsRNA, non-capped RNA and/or RNA fragments.

In embodiments, the coding RNA of the disclosure has a certain RNA integrity.

The term “RNA integrity” generally describes whether the complete RNA sequence is present. Low RNA integrity could be due to, amongst others, RNA degradation, RNA cleavage, incorrect or incomplete chemical synthesis of the RNA, incorrect base pairing, integration of modified nucleotides or the modification of already integrated nucleotides, lack of capping or incomplete capping, lack of polyadenylation or incomplete polyadenylation, or incomplete RNA in vitro transcription. RNA is a fragile molecule that can easily degrade, which may be caused e.g. by temperature, ribonucleases, pH or other factors (e.g. nucleophilic attacks, hydrolysis etc.), which may reduce the RNA integrity and, consequently, its functionality.

The skilled person can choose from a variety of different chromatographic or electrophoretic methods for determining integrity of RNA. Chromatographic and electrophoretic (e.g. capillary gel electrophoresis) methods are well-known in the art. In case chromatography is used (e.g. RP-HPLC), the analysis of the integrity of the RNA may be based on determining the peak area (or “area under the peak”) of the expected full length RNA (the RNA with the correct RNA length) in a corresponding chromatogram.

In embodiments, the coding RNA of the disclosure has an RNA integrity ranging from about 40% to about 100%. In embodiments, the RNA has an RNA integrity ranging from about 50% to about 100%. In embodiments, the RNA has an RNA integrity ranging from about 60% to about 100%. In embodiments, the RNA has an RNA integrity ranging from about 70% to about 100%. In embodiments, the RNA integrity is for example about 50%, about 60%, about 70%, about 80%, or about 90%. RNA is suitably determined using analytical HPLC, for example analytical RP-HPLC.

In some embodiments, the coding RNA has an RNA integrity of at least about 50%, for example of at least about 60%, for example of at least about 70%, for example of at least about 80% or about 90%. RNA integrity is suitably determined using analytical HPLC, for example analytical RP-HPLC.

In some embodiments, the coding RNA is suitable for intranasal, oral, sublingual, intravenous, intramuscular, intradermal, transdermal, or subcutaneous administration. In some embodiments, the coding RNA is suitable for intramuscular administration.

### RNA Constructs:

In some embodiments, the coding RNA comprises at least the following elements, for example in 5′ to 3′ direction:
A) a 5′-cap structure, for example as specified herein;
B) at least one cds encoding an antigenic polypeptide which is selected or derived from Escherichia coli FimH as defined herein;
C) a 5′-UTR and/or a 3′-UTR, for example as specified herein;
D) at least one poly(A) sequence, for example as specified herein.

In various embodiments, the coding RNA, for example the mRNA, comprises the following elements, for example in 5′ to 3′ direction:
A) a 5′-cap structure, for example as specified herein;
B) a 5′-terminal start element, for example as specified herein;
C) optionally, a 5′-UTR, for example as specified herein;
D) a ribosome binding site, for example as specified herein;
E) at least one cds encoding an antigenic polypeptide which is selected or derived from Escherichia coli FimH as defined herein;
F) a 3′-UTR, for example as specified herein;
G) optionally, at least one poly(A) sequence, for example as specified herein;
H) optionally, at least one poly(C) sequence, for example as specified herein;
I) optionally, histone stem-loop for example as specified herein;
J) optionally, 3′-terminal sequence element, for example as specified herein;
K) optionally, chemically modified nucleotides, for example as specified herein.

In various embodiments, the coding RNA, for example the mRNA, comprises the following elements, for example in 5′ to 3′ direction:
A) a 5′-cap structure;
B) a 5′-UTR for example selected or derived from a 5′-UTR of a HSD17B4 gene;
C) at least one coding sequence encoding an antigenic polypeptide which is selected or derived from Escherichia coli FimH as defined herein;
D) a 3′-UTR for example selected or derived from a 3′-UTR of a PSMB3 gene;
E) optionally, a histone stem-loop; and
F) a poly(A) sequence for example comprising about 100 A nucleotides.

In some embodiments, the mRNA comprises the following elements for example in 5′- to 3′-direction:
A) a cap1 structure for example as defined herein;
B) a 5′-UTR derived from a HSD17B4 gene as defined herein;
C) at least one cds encoding an antigenic polypeptide which is selected or derived from Escherichia coli FimH as defined herein, for example wherein the coding sequence that comprises at least one of the nucleic acid sequences being identical or at least 70%, 80%, 85%, 86%, 87%, 88%, 89%, 90%, 91%, 92%, 93%, 94%, 95%, 96%, 97%, 98%, or 99% identical to the sequences according to any one of SEQ ID NOs: 187-246, 257-316, 523-545, 548-570, 573-595, 598-620, 623-645, 648-670, or a fragment or a fragment or variant thereof;
D) a 3′-UTR derived from a 3′-UTR of a PSMB3 gene as defined herein;
E) optionally, a histone stem-loop as defined herein;
F) a poly(A) sequence for example comprising about 100 A nucleotides;
G) optionally, chemically modified nucleotides, e.g. pseudouridine (ψ) or N1-methylpseudouridine (m1ψ).

In further embodiments, the mRNA comprises the following elements for example in 5′- to 3′-direction:
A) a cap1 structure for example as defined herein;
B) a 5′-UTR derived from a HSD17B4 gene as defined herein;
C) at least one cds encoding an antigenic polypeptide which is selected or derived from Escherichia coli FimH as defined herein, wherein the coding sequence that comprises at least one of the nucleic acid sequences being identical or at least 70%, 80%, 85%, 86%, 87%, 88%, 89%, 90%, 91%, 92%, 93%, 94%, 95%, 96%, 97%, 98%, or 99% identical to the sequences according to any one of SEQ ID NOs: 523-545, 548-570, 573-595, 598-620, 623-645, 648-670, or a fragment or a fragment or variant thereof;
D) a 3′-UTR derived from a 3′-UTR of a PSMB3 gene as defined herein;
E) optionally, a histone stem-loop as defined herein;
F) a poly(A) sequence for example comprising about 100 A nucleotides;
G) optionally, chemically modified nucleotides, e.g. pseudouridine (ψ) or N1-methylpseudouridine (m1ψ).

In some embodiments, the mRNA comprises the following elements in 5′- to 3′-direction:
A) a cap1 structure as defined herein;
B) a 5′-UTR derived from a HSD17B4 gene as defined herein;
C) at least one cds encoding an antigenic polypeptide which is or is derived from Escherichia coli FimH as defined herein, for example wherein the coding sequence that comprises at least one of the nucleic acid sequences being identical or at least 70%, 80%, 85%, 86%, 87%, 88%, 89%, 90%, 91%, 92%, 93%, 94%, 95%, 96%, 97%, 98%, or 99% identical to the sequences according to any one of SEQ ID NOs: 529, 554, 579, 604, 629, 654, or a fragment or a fragment or variant thereof;
D) a 3′-UTR derived from a 3′-UTR of a PSMB3 gene as defined herein;
E) optionally, a histone stem-loop as defined herein;
F) a poly(A) sequence comprising about 100 A nucleotides, for example representing the 3′ terminus;
G) optionally, chemically modified nucleotides, e.g. pseudouridine (ψ) or N1-methylpseudouridine (m1ψ).

In some embodiments, the mRNA comprises the following elements in 5′- to 3′-direction:
A) a cap1 structure as defined herein;
B) a 5′-UTR derived from a HSD17B4 gene as defined herein;
C) at least one cds encoding an antigenic polypeptide which is or is derived from Escherichia coli FimH as defined herein, for example wherein the coding sequence that comprises at least one of the nucleic acid sequences being identical or at least 70%, 80%, 85%, 86%, 87%, 88%, 89%, 90%, 91%, 92%, 93%, 94%, 95%, 96%, 97%, 98%, or 99% identical to the sequences according to any one of SEQ ID NOs: 533, 558, 583, 608, 633, 658, or a fragment or a fragment or variant thereof;
D) a 3′-UTR derived from a 3′-UTR of a PSMB3 gene as defined herein;
E) optionally, a histone stem-loop as defined herein;
F) a poly(A) sequence comprising about 100 A nucleotides, for example representing the 3′ terminus;
G) optionally, chemically modified nucleotides, e.g. pseudouridine (ψ) or N1-methylpseudouridine (m1ψ).

In some embodiments, the mRNA comprises the following elements in 5′- to 3′-direction:
A) a cap1 structure as defined herein;
B) a 5′-UTR derived from a HSD17B4 gene as defined herein;
C) at least one cds encoding an antigenic polypeptide which is or is derived from Escherichia coli FimH as defined herein, for example wherein the coding sequence that comprises at least one of the nucleic acid sequences being identical or at least 70%, 80%, 85%, 86%, 87%, 88%, 89%, 90%, 91%, 92%, 93%, 94%, 95%, 96%, 97%, 98%, or 99% identical to the sequences according to any one of SEQ ID NOs: 534, 559, 584, 609, 634, 659, or a fragment or a fragment or variant thereof;
D) a 3′-UTR derived from a 3′-UTR of a PSMB3 gene as defined herein;
E) optionally, a histone stem-loop as defined herein;
F) a poly(A) sequence comprising about 100 A nucleotides, for example representing the 3′ terminus;
G) optionally, chemically modified nucleotides, e.g. pseudouridine (ψ) or N1-methylpseudouridine (m1ψ).

RNA sequences are provided in Table 2. Therein, each row represents a specific suitable RNA construct of the disclosure (compare with Table 1), wherein the description of the construct is indicated in column A of Table 2 and the SEQ ID NOs of the amino acid sequence of the respective construct is provided in column B. The corresponding SEQ ID NOs of the coding sequences encoding the respective constructs are provided in Table 1.

The corresponding RNA sequences, in particular mRNA sequences are provided in columns C and D, wherein column C provides RNA sequences with an UTR combination “HSD17B4/PSMB3” and a 3′-terminal hSL-A100 tail and wherein column 0 provides nucleic acid sequences with an UTR combination “HSD17B4/PSMB3” and 3′-terminal A100 tail.

TABLE 2
RNA constructs.

| row | A | B | C | D |
|---|---|---|---|---|
| 1 | Ec(J96)-FimC(1-36)_ | 496 | 671, 696, | 971, 996, |
| | Ec(J96)-FimC(37-241); | | 821, 846 | 1121, 1146 |
| | construct #1 | | | |
| 2 | HslgE(1-18)_Ec(J96)- | 497 | 672, 697, | 972, 997, |
| | FimC(37-241); construct #2 | | 822, 847 | 1122, 1147 |
| 3 | Ec(J96)-FimH(1-21)_ | 498 | 673, 698, | 973, 998, |
| | Ec(J96)-FimH(22-300); | | 823, 848 | 1123, 1148 |
| | construct #3 | | | |
| 4 | HslgE(1-18)_Ec(J96)- | 499 | 674, 699, | 974, 999, |
| | FimH(22-300); construct #4 | | 824, 849 | 1124, 1149 |
| 5 | MmlgK(1-20)_Ec(J96)- | 500 | 675, 700, | 975, 1000, |
| | FimH(22-300); construct #5 | | 825, 850 | 1125, 1150 |
| 6 | Ec(J96)-FimH(1-21)_Ec(J96)- | 501 | 676, 701, | 976, 1001, |
| | FimH(22-300)_Linker- | | 826,851 | 1126, 1151 |
| | PGDGN_Ec(J96)- | | | |
| | FimG(24-37); construct #6 | | | |
| 7 | HslgE(1-18)_Ec(J96)- | 502 | 677, 702, | 977, 1002, |
| | FimH(22-300)_Linker- | | 827, 852 | 1127, 1152 |
| | PGDGN_Ec(J96)- | | | |
| | FimG(24-37); construct #7 | | | |
| 8 | HslgE(1-18)_Ec(J96)- | 503 | 678, 703, | 978, 1003, |
| | FimH(22-300)_Linker- | | 828, 853 | 1128, 1153 |
| | G4SGG_Ec(J96)- | | | |
| | FimG(24-37); construct #8 | | | |
| 9 | MmlgK(1-20)_Ec(J96)- | 504 | 679, 704, | 979, 1004, |
| | FimH(22-300)_Linker- | | 829, 854, | 1129, 1154 |
| | PGDGN_Ec(J96)- | | 1271, | |
| | FimG(24-37); construct #9 | | 1274 | |
| 10 | MmlgK(1-20)_Ec(536)- | 505 | 680, 705, | 980, 1005, |
| | FimH(22-300)_Linker- | | 830, 855 | 1130, 1155 |
| | PGDGN_Ec(J96)- | | | |
| | FimG(24-37); construct #10 | | | |
| 11 | HslgE(1-18)_Ec(J96)- | 506 | 681, 706, | 981, 1006, |
| | FimH(22-300)_Linker- | | 831, 856 | 1131, 1156 |
| | (G4S)3_Ec(J96)- | | | |
| | FimC(135-148); construct #11 | | | |
| 12 | LumSynth(1-154_C37A_ | 507 | 682, 707, | 982, 1007, |
| | N102D)_Linker(GGS)4- | | 832, 857 | 1132, 1157 |
| | GGG_Ec(J96)- | | | |
| | FimH(22-300)_Linker- | | | |
| | PGDGN_Ec(J96)- | | | |
| | FimG(24-37); construct #12 | | | |
| 13 | HslgE(1-18)_Ec(J96)- | 508 | 683, 708, | 983, 1008, |
| | FimH(22-300)_Linker- | | 833, 858, | 1133, 1158 |
| | PGDGN_Ec(J96)- | | 1272, | |
| | FimG(24-37)_Linker- | | 1275 | |
| | SGG_LumSynth(1-154_ | | | |
| | C37A_N102D); construct #13 | | | |
| 14 | HslgE(1-18)_Ec(J96)- | 509 | 684, 709, | 984, 1009, |
| | FimH(22-300)_Linker- | | 834, 859, | 1134, 1159 |
| | PGDGN_Ec(J96)- | | 1273, | |
| | FimG(24-37)_Linker- | | 1276 | |
| | SGG_Ferritin(5-167_N19Q); | | | |
| | construct #14 | | | |
| 15 | MmlgK(1-20)_Ec(J96)- | 510 | 685, 710, | 985, 1010, |
| | FimH(22-300)_Linker- | | 835, 860 | 1135, 1160 |
| | PGDGN_Ec(J96)- | | | |
| | FimG(24-37)_Linker- | | | |
| | (GGS)4_Ferritin(5-166); | | | |
| | construct #15 | | | |
| 16 | HslgE(1-18)_Ec(J96)- | 511 | 686, 711, | 986, 1011, |
| | FimH(22-300)_Linker- | | 836, 861 | 1136, 1161 |
| | PGDGN_Ec(J96)-FimG(24- | | | |
| | 37)_TMdomain-HA(510- | | | |
| | 566); construct #16 | | | |
| 17 | HslgE(1-18)_LumSynth(1- | 512 | 687, 712, | 987, 1012, |
| | 154_C37A_N102D)_ | | 837, 862 | 1137, 1162 |
| | Linker(GGS)4-GGG_Ec(J96)- | | | |
| | FimH(22-300)_Linker- | | | |
| | PGDGN_Ec(J96)-FimG(24-37); | | | |
| | construct #17 | | | |
| 18 | HslgE(1-18)_Ec(J96)-FimH(22- | 513 | 688, 713, | 988, 1013, |
| | 300_N28S_N91S_N249Q)_ | | 838, 863 | 1138, 1163 |
| | Linker-PGDGN_Ec(J96)- | | | |
| | FimG(24-37)_Linker- | | | |
| | SGG_LumSynth(1- | | | |
| | 154_C37A_N102D); | | | |
| | construct #18 | | | |
| 19 | HslgE(1-18)_Ec(J96)-FimH(22- | 514 | 689, 714, | 989, 1014, |
| | 300_N28S_N91S_ | | 839, 864 | 1139, 1164 |
| | N249Q_N256D)_Linker- | | | |
| | PGDGN_Ec(J96)-FimG(24-37)_ | | | |
| | Linker-SGG_LumSynth(1-154_ | | | |
| | C37A_N102D); construct #19 | | | |
| 20 | HslgE(1-18)_Ec(J96)-FimH(22- | 515 | 690, 715, | 990, 1015, |
| | 300_N28S_V48C_L55C_N91S_ | | 840, 865 | 1140, 1165 |
| | N249Q)_Linker-PGDGN_Ec(J96)- | | | |
| | FimG(24-37)_Linker- | | | |
| | SGG_LumSynth(1-154_ | | | |
| | C37A_N102D); construct #20 | | | |
| 21 | HslgE(1-18)_Ec(J96)- | 516 | 691, 716, | 991, 1016, |
| | FimH(22-300_F165V)_Linker- | | 841, 866 | 1141, 1166 |
| | PGDGN_Ec(J96)- | | | |
| | FimG(24-37)_Linker- | | | |
| | SGG_LumSynth(1-154_ | | | |
| | C37A_N102D); construct #21 | | | |
| 22 | HslgE(1-18)_Ec(J96)-FimH(22- | 517 | 692, 717, | 992, 1017, |
| | 300_N28S_N91S_N249Q)_ | | 842, 867 | 1142, 1167 |
| | Linker-PGDGN_Ec(J96)- | | | |
| | FimG(24-37)_Linker-SGG_ | | | |
| | Ferritin(5-167_N19Q); | | | |
| | construct #22 | | | |
| 23 | HslgE(1-18)_Ec(J96)-FimH(22- | 518 | 693, 718, | 993, 1018, |
| | 300_N28S_N91S_ | | 843, 868 | 1143, 1168 |
| | N249Q_N256D)_Linker- | | | |
| | PGDGN_Ec(J96)- | | | |
| | FimG(24-37)_Linker- | | | |
| | SGG_Ferritin(5-167_N19Q); | | | |
| | construct #23 | | | |
| 24 | HslgE(1-18)_Ec(J96)-FimH(22- | 519 | 694, 719, | 994, 1019, |
| | 300_N28S_V48C_L55C_ | | 844, 869 | 1144, 1169 |
| | N91S_N249Q)_Linker- | | | |
| | PGDGN_Ec(J96)-FimG(24- | | | |
| | 37)_Linker-SGG_Ferritin(5- | | | |
| | 167_N19Q); construct #24 | | | |
| 25 | HslgE(1-18)_Ec(J96)- | 520 | 695, 720, | 995, 1020, |
| | FimH(22-300_F165V)_Linker- | | 845, 870 | 1145, 1170 |
| | PGDGN_Ec(J96)-FimG(24- | | | |
| | 37)_Linker-SGG_Ferritin(5- | | | |
| | 167_N19Q); construct #25 | | | |

Ec: Escherichia coli; HA: Hemagglutinin; Hs: Homo sapiens; IgE: immunoglobulin E; IgK: immunoglobulin Kappa; LumSynth, LS: Lumazine synthase; Mm: Mus musculus; TMdomain, TM: transmembrane domain

In some embodiments, the coding RNA comprises or consists of a nucleic acid sequence which is identical or at least 70%, 80%, 85%, 86%, 87%, 88%, 89%, 90%, 91%, 92%, 93%, 94%, 95%, 96%, 97%, 98%, or 99% identical to a nucleic acid sequence according to any one of SEQ ID NOs: 673-695, 698-720, 723-745, 748-770, 773-795, 798-820, 823-845, 848-870, 873-895, 898-920, 923-945, 948-970, 973-995, 998-1020, 1023-1045, 1048-1070, 1073-1095, 1098-1120, 1123-1145, 1148-1170, 1173-1195, 1198-1220, 1223-1245, 1248-1270 or a fragment or variant thereof.

In further embodiments, the coding RNA comprises or consists of a nucleic acid sequence which is identical or at least 70%, 80%, 85%, 86%, 87%, 88%, 89%, 90%, 91%, 92%, 93%, 94%, 95%, 96%, 97%, 98%, or 99% identical to a nucleic acid sequence according to any one of SEQ ID NOs: 673-695, 698-720, 723-745, 748-770, 773-795, 798-820, 823-845, 848-870, 873-895, 898-920, 923-945, 948-970, 973-995, 998-1020, 1023-1045, 1048-1070, 1073-1095, 1098-1120, 1123-1145, 1148-1170, 1173-1195, 1198-1220, 1223-1245, 1248-1270 or a fragment or variant thereof, wherein at least one, for example all uracil nucleotides in said RNA sequences are replaced by pseudouridine (ψ) nucleotides and/or N1-methylpseudouridine (m1ψ) nucleotides. In one embodiment, the coding RNA comprises or consists of a nucleic acid sequence which is identical or at least 70%, 80%, 85%, 86%, 87%, 88%, 89%, 90%, 91%, 92%, 93%, 94%, 95%, 96%, 97%, 98%, or 99% identical to a nucleic acid sequence according to any one of SEQ ID NOs: 1271-1273 or a fragment or variant thereof, wherein all uracil nucleotides in said RNA sequences are replaced by N1-methylpseudouridine (m1ψ) nucleotides. In one embodiment, the coding RNA comprises or consists of a nucleic acid sequence which is identical or at least 70%, 80%, 85%, 86%, 87%, 88%, 89%, 90%, 91%, 92%, 93%, 94%, 95%, 96%, 97%, 98%, or 99% identical to a nucleic acid sequence according to any one of SEQ ID NOs: 1274-1276 or a fragment or variant thereof, wherein all uracil nucleotides in said RNA sequences are replaced by pseudouridine (ψ) nucleotides.

In one embodiment, the coding RNA comprises or consists of a nucleic acid sequence which is identical or at least 70%, 80%, 85%, 86%, 87%, 88%, 89%, 90%, 91%, 92%, 93%, 94%, 95%, 96%, 97%, 98%, or 99% identical to a nucleic acid sequence according to any one of SEQ ID NOs: 679, 704, 729, 754, 779, 804, 829, 854, 879, 904, 929, 954, 979, 1004, 1029, 1054, 1079, 1104, 1129, 1154, 1179, 1204, 1229, 1254 or a fragment or variant thereof. In that embodiment, said RNA sequences optionally comprise a 5′-terminal cap1 structure. In that embodiment, said RNA sequences do optionally not comprise chemically modified nucleotides.

In one embodiment, the coding RNA comprises or consists of a nucleic acid sequence which is identical or at least 70%, 80%, 85%, 86%, 87%, 88%, 89%, 90%, 91%, 92%, 93%, 94%, 95%, 96%, 97%, 98%, or 99% identical to a nucleic acid sequence according to any one of SEQ ID NOs: 683, 708, 733, 758, 783, 808, 833, 858, 883, 908, 933, 958, 983, 1008, 1033, 1058, 1083, 1108, 1133, 1158, 1183, 1208, 1233, 1258 or a fragment or variant thereof. In that embodiment, said RNA sequences optionally comprise a 5′-terminal cap1 structure. In that embodiment, said RNA sequences do optionally not comprise chemically modified nucleotides.

In one embodiment, the coding RNA comprises or consists of a nucleic acid sequence which is identical or at least 70%, 80%, 85%, 86%, 87%, 88%, 89%, 90%, 91%, 92%, 93%, 94%, 95%, 96%, 97%, 98%, or 99% identical to a nucleic acid sequence according to any one of SEQ ID NOs: 684, 709, 734, 759, 784, 809, 834, 859, 884, 909, 934, 959, 984, 1009, 1034, 1059, 1084, 1109, 1134, 1159, 1184, 1209, 1234, 1259 or a fragment or variant thereof. In that embodiment, said RNA sequences optionally comprise a 5′-terminal cap1 structure. In that embodiment, said RNA sequences do optionally not comprise chemically modified nucleotides.

In one embodiment, the coding RNA comprises or consists of a nucleic acid sequence which is identical or at least 70%, 80%, 85%, 86%, 87%, 88%, 89%, 90%, 91%, 92%, 93%, 94%, 95%, 96%, 97%, 98%, or 99% identical to a nucleic acid sequence according to any one of SEQ ID NOs: 679, 704, 729, 754, 779, 804, 829, 854, 879, 904, 929, 954, 979, 1004, 1029, 1054, 1079, 1104, 1129, 1154, 1179, 1204, 1229, 1254 or a fragment or variant thereof. In that embodiment, said RNA sequences optionally comprise a 5′-terminal cap1 structure. In that embodiment, said RNA sequences optionally comprise pseudouridine (ψ) nucleotides.

In one embodiment, the coding RNA comprises or consists of a nucleic acid sequence which is identical or at least 70%, 80%, 85%, 86%, 87%, 88%, 89%, 90%, 91%, 92%, 93%, 94%, 95%, 96%, 97%, 98%, or 99% identical to a nucleic acid sequence according to any one of SEQ ID NOs: 683, 708, 733, 758, 783, 808, 833, 858, 883, 908, 933, 958, 983, 1008, 1033, 1058, 1083, 1108, 1133, 1158, 1183, 1208, 1233, 1258 or a fragment or variant thereof. In that embodiment, said RNA sequences optionally comprise a 5′-terminal cap1 structure. In that embodiment, said RNA sequences optionally comprise pseudouridine (ψ) nucleotides.

In one embodiment, the coding RNA comprises or consists of a nucleic acid sequence which is identical or at least 70%, 80%, 85%, 86%, 87%, 88%, 89%, 90%, 91%, 92%, 93%, 94%, 95%, 96%, 97%, 98%, or 99% identical to a nucleic acid sequence according to any one of SEQ ID NOs: 684, 709, 734, 759, 784, 809, 834, 859, 884, 909, 934, 959, 984, 1009, 1034, 1059, 1084, 1109, 1134, 1159, 1184, 1209, 1234, 1259 or a fragment or variant thereof. In that embodiment, said RNA sequences optionally comprise a 5′-terminal cap1 structure. In that embodiment, said RNA sequences optionally comprise pseudouridine (ψ) nucleotides.

In one embodiment, the coding RNA comprises or consists of a nucleic acid sequence which is identical or at least 70%, 80%, 85%, 86%, 87%, 88%, 89%, 90%, 91%, 92%, 93%, 94%, 95%, 96%, 97%, 98%, or 99% identical to a nucleic acid sequence according to any one of SEQ ID NOs: 679, 704, 729, 754, 779, 804, 829, 854, 879, 904, 929, 954, 979, 1004, 1029, 1054, 1079, 1104, 1129, 1154, 1179, 1204, 1229, 1254 or a fragment or variant thereof. In that embodiment, said RNA sequences optionally comprise a 5′-terminal cap1 structure. In that, said RNA sequences optionally comprise N1-methylpseudouridine (m1ψ) nucleotides.

In one embodiment, the coding RNA comprises or consists of a nucleic acid sequence which is identical or at least 70%, 80%, 85%, 86%, 87%, 88%, 89%, 90%, 91%, 92%, 93%, 94%, 95%, 96%, 97%, 98%, or 99% identical to a nucleic acid sequence according to any one of SEQ ID NOs: 683, 708, 733, 758, 783, 808, 833, 858, 883, 908, 933, 958, 983, 1008, 1033, 1058, 1083, 1108, 1133, 1158, 1183, 1208, 1233, 1258 or a fragment or variant thereof. In that embodiment, said RNA sequences optionally comprise a 5′-terminal cap1 structure. In that embodiment, said RNA sequences optionally comprise N1-methylpseudouridine (m1ψ) nucleotides.

In one embodiment, the coding RNA comprises or consists of a nucleic acid sequence which is identical or at least 70%, 80%, 85%, 86%, 87%, 88%, 89%, 90%, 91%, 92%, 93%, 94%, 95%, 96%, 97%, 98%, or 99% identical to a nucleic acid sequence according to any one of SEQ ID NOs: 684, 709, 734, 759, 784, 809, 834, 859, 884, 909, 934, 959, 984, 1009, 1034, 1059, 1084, 1109, 1134, 1159, 1184, 1209, 1234, 1259 or a fragment or variant thereof. In that embodiment, said RNA sequences optionally comprise a 5′-terminal cap1 structure. In that embodiment, said RNA sequences optionally comprise N1-methylpseudouridine (m1ψ) nucleotides.

In other embodiments, the coding RNA of the disclosure is a 5′ capped (cap1) mRNA that comprises or consists of an RNA sequence consisting of non-modified ribonucleotides (A, G, C, U) which is identical to an RNA sequence according to any one of SEQ ID NOs: 679, 829, 979, 1129 or a fragment or variant thereof.

In other embodiments, the coding RNA of the disclosure is a 5′ capped (cap1) mRNA that comprises or consists of an RNA sequence consisting of non-modified ribonucleotides (A, G, C, U) which is identical to an RNA sequence according to any one of SEQ ID NOs: 683, 833, 983, 1133, or a fragment or variant thereof.

In other embodiments, the coding RNA of the disclosure is a 5′ capped (cap1) mRNA that comprises or consists of an RNA sequence consisting of non-modified ribonucleotides (A, G, C, U) which is identical to an RNA sequence according to any one of SEQ ID NOs: 684, 834, 984, 1134, or a fragment or variant thereof.

In one embodiment, the coding RNA of the disclosure is a 5′ capped (cap1) mRNA that comprises or consists of an RNA sequence consisting of non-modified ribonucleotides (A, G, C) and chemically modified pseudouridine (ψ) ribonucleotides which is identical to an RNA sequence according to any one of SEQ ID NOs: 679, 829, 979, 1129, 1274 or a fragment or thereof.

In one embodiment, the coding RNA of the disclosure is a 5′ capped (cap1) mRNA that comprises or consists of an RNA sequence consisting of non-modified ribonucleotides (A, G, C) and chemically modified pseudouridine (ψ) ribonucleotides which is identical to an RNA sequence according to any one of SEQ ID NOs: 683, 833, 983, 1133, 1275 or a fragment or variant thereof.

In one embodiment, the coding RNA of the disclosure is a 5′ capped (cap1) mRNA that comprises or consists of an RNA sequence consisting of non-modified ribonucleotides (A, G, C) and chemically modified pseudouridine (ψ) ribonucleotides which is identical to an RNA sequence according to any one of SEQ ID NOs: 684, 834, 984, 1134, 1276 or a fragment or variant thereof.

In one embodiment, the coding RNA of the disclosure is a 5′ capped (cap1) mRNA that comprises or consists of an RNA sequence consisting of non-modified ribonucleotides (A, G, C) and chemically modified N1-methylpseudouridine (m1ψ) ribonucleotides which is identical to an RNA sequence according to any one of SEQ ID NOs: 679, 829, 979, 1129, 1271 or a fragment or variant thereof. In one embodiment, the coding RNA of the disclosure is a 5′ capped (cap1) mRNA that comprises or consists of an RNA sequence consisting of non-modified ribonucleotides (A, G, C) and chemically modified N1-methylpseudouridine (m1ψ) ribonucleotides which is identical to an RNA sequence according to any one of SEQ ID NOs: 683, 833, 983, 1133, 1272 or a fragment or variant thereof.

In one embodiment, the coding RNA of the disclosure is a 5′ capped (cap1) mRNA that comprises or consists of an RNA sequence consisting of non-modified ribonucleotides (A, G, C) and chemically modified N1-methylpseudouridine (m1ψ) ribonucleotides which is identical to an RNA sequence according to any one of SEQ ID NOs: 684, 834, 984, 1134, 1273 or a fragment or variant thereof.

2: Composition Comprising a Coding RNA Encoding an Antigenic Polypeptide of Escherichia coli FimH

In a second aspect, it is provided a pharmaceutical composition comprising the coding RNA of the first aspect.

Notably, embodiments relating to the pharmaceutical composition of the second aspect may likewise be read on and be understood as suitable embodiments of the vaccine of the third aspect. Also, embodiments relating to the vaccine of the third aspect may likewise be read on and be understood as suitable embodiments of the pharmaceutical composition of the second aspect. Furthermore, features and embodiments described in the context of the first aspect (the RNA of the disclosure) have to be read on and have to be understood as suitable embodiments of the pharmaceutical composition of the second aspect.

In the context of the disclosure, a “composition” refers to any type of composition in which the specified ingredients (e.g. coding RNA comprising (a) at least one untranslated region (UTR); and (b) a coding sequence operably linked to said UTR encoding an antigenic polypeptide which is selected or derived from Escherichia coli FimH) may be incorporated, optionally along with any further constituents, usually with at least one pharmaceutically acceptable carrier or excipient. The composition may be a dry composition such as a powder, a granule, or a solid lyophilized form. Alternatively, the composition may be in liquid form, and each constituent may be independently incorporated in dissolved or dispersed (e.g. suspended or emulsified) form.

In various embodiments, the coding RNA of the pharmaceutical composition is selected from a coding RNA as defined in any of the embodiments of the first aspect.

In embodiments, the coding RNA as comprised in the pharmaceutical composition is provided in an amount of at least about 100 ng to up to about 500 μg, in an amount of at least about 1 μg to up to about 200 μg, in an amount of about at least 1 μg to up to about 100 μg, in an amount of at least about 5 μg to up to about 100 μg, for example in an amount of at least about 10 μg to up to about 50 μg, specifically, in an amount of about 1 μg, 2 μg, 3 μg, 4 μg, 5 μg, 6 μg, 7 μg, 8 μg, 9 μg, 10 μg, 11 μg, 12 μg, 13 μg, 14 μg, 15 μg, 20 μg, 25 μg, 30 μg, 35 μg, 40 μg, 45 μg, 50 μg, 55 μg, 60 μg, 65 μg, 70 μg, 75 μg, 80 μg, 85 μg, 90 μg, 95 μg or 100 μg.

In one embodiment, the coding RNA of the composition comprises or consists of an RNA sequence identical or at least 70%, 80%, 90%, 91%, 92%, 93%, 94%, 95%, 96%, 97%, 98%, or 99% identical to any one of SEQ ID NOs: 673-695, 698-720, 723-745, 748-770, 773-795, 798-820, 823-845, 848-870, 873-895, 898-920, 923-945, 948-970, 973-995, 998-1020, 1023-1045, 1048-1070, 1073-1095, 1098-1120, 1123-1145, 1148-1170, 1173-1195, 1198-1220, 1223-1245, 1248-1270, 1271-1276 or a fragment or a variant thereof.

In some embodiments, the pharmaceutical composition comprises a plurality or at least more than one RNA species.

In one embodiment, the pharmaceutical composition comprises a first coding RNA according to the first aspect and a second coding RNA encoding a polypeptide which is selected or derived from Escherichia coli FimC.

In embodiments, the second coding sequence encodes an FimC amino acid sequence which is identical or at least 70%, 80%, 85%, 86%, 87%, 88%, 89%, 90%, 91%, 92%, 93%, 94%, 95%, 96%, 97%, 98%, or 99% identical to any one of SEQ ID NOs: 317, 324, 496, 497, or a fragment or variant thereof. This is particularly suitable when the first coding sequence encodes a FimH amino acid sequence which is identical or at least 70%, 80%, 85%, 86%, 87%, 88%, 89%, 90%, 91%, 92%, 93%, 94%, 95%, 96%, 97%, 98%, or 99% identical to any one of SEQ ID NOs: 177-186, 247-256, particularly SEQ ID NOs: 498, 500 or is an immunogenic fragment or immunogenic variant thereof.

In embodiments, the second coding RNA comprises a FimC coding sequence that comprises at least one of the nucleic acid sequences being identical or at least 70%, 80%, 85%, 86%, 87%, 88%, 89%, 90%, 91%, 92%, 93%, 94%, 95%, 96%, 97%, 98%, or 99% identical to the sequences according to any one of SEQ ID NOs: 318-323, 325-330, 521, 522, 546, 547, 571, 572, 596, 597, 621, 622, 646, 647, or a fragment or variant thereof. This is particularly suitable when the first coding sequence comprises a FimH coding sequence that comprises at least one of the nucleic acid sequences being identical or at least 70%, 80%, 85%, 86%, 87%, 88%, 89%, 90%, 91%, 92%, 93%, 94%, 95%, 96%, 97%, 98%, or 99% identical to any one of SEQ ID NOs: 187-246, 257-316, 523, 525, 548, 550, 573, 575, 598, 600, 623, 625, 648, 650.

In embodiments, the second coding RNA comprises or consists of a FimC coding nucleic acid sequence which is identical or at least 70%, 80%, 85%, 86%, 87%, 88%, 89%, 90%, 91%, 92%, 93%, 94%, 95%, 96%, 97%, 98%, or 99% identical to a nucleic acid sequence according to any one of SEQ ID NOs: 671, 672, 696, 697, 721, 722, 746, 747, 771, 772, 796, 797, 821, 822, 846, 847, 871, 872, 896, 897, 921, 922, 946, 947, 971, 972, 996, 997, 1021, 1022, 1046, 1047, 1071, 1072, 1096, 1097, 1121, 1122, 1146, 1147, 1171, 1172, 1196, 1197, 1221, 1222, 1246, 1247, or a fragment or variant of any of these sequences, optionally wherein at least one, for example all uracil nucleotides in said RNA sequences are replaced by pseudouridine (ψ) nucleotides and/or N1-methylpseudouridine (m1ψ) nucleotides.

Notably, suitable nucleic acid features (e.g. UTRs, cap structures, modifications, codon optimizations) that are disclosed in the context of FimH encoding RNA sequences of the first aspect may also apply and may also be suitable for FimC encoding RNA sequences of second aspect.

Specific features and embodiments relating to the coding RNA of the first aspect as provided herein may also apply for the second coding RNA encoding FimC. Providing a pharmaceutical composition comprising a first coding RNA encoding FimH and a second coding RNA encoding FimC is particularly suitable so that, once the FimH and FimC polypeptides encoded by the first and second coding RNA are translated, they can assemble in a non-covalent complex. This is particularly suitable in order to stabilise FimH when the coding sequence of the first RNA encoding FimH does not encode a donor strand peptide.

E. coli FimC sequences and constructs are reported in Table 1 and Table 2.

In various embodiments, the coding RNA of the pharmaceutical composition is formulated with a pharmaceutically acceptable carrier or excipient.

The term “pharmaceutically acceptable carrier” or “pharmaceutically acceptable excipient” as used herein for example includes the liquid or non-liquid basis of the composition for administration. If the composition is provided in liquid form, the carrier may be water, e.g. pyrogen-free water; isotonic saline or buffered (aqueous) solutions, e.g. phosphate, citrate etc. buffered solutions. Water or for example a buffer, for example an aqueous buffer, may be used, comprising e.g. a sodium salt, a calcium salt, or a potassium salt. According to some embodiments, the sodium, calcium or potassium salts may occur in the form of their halogenides, e.g. chlorides, iodides, or bromides, in the form of their hydroxides, carbonates, hydrogen carbonates, or sulphates, etc. Examples of sodium salts include NaCl, Na₂HPO₄, NaI, NaBr, Na₂CO₃, NaHCO₃, Na₂SO₄, examples of the optional potassium salts include KCl, KI, KBr, K₂CO₃, KHCO₃, K₂SO₄, and examples of calcium salts include CaCl₂, CaI₂, CaBr₂, CaCO₃, CaSO₄, Ca(OH)₂.

Furthermore, organic anions of the aforementioned cations may be in the buffer. Accordingly, in embodiments, the pharmaceutical composition may comprise pharmaceutically acceptable carriers or excipients using one or more pharmaceutically acceptable carriers or excipients to e.g. increase stability, increase cell transfection, permit the sustained or delayed, increase the translation of encoded antigenic polypeptide in vivo, and/or alter the release profile of encoded antigenic peptide in vivo. In addition to traditional excipients such as any and all solvents, dispersion media, diluents, or other liquid vehicles, dispersion or suspension aids, surface active agents, isotonic agents, thickening or emulsifying agents, preservatives, excipients of the present disclosure can include, without limitation, lipidoids, liposomes, lipid nanoparticles, polymers, lipoplexes, core-shell nanoparticles, peptides, proteins, cells transfected with polynucleotides, hyaluronidase, nanoparticle mimics and combinations thereof. In embodiments, one or more compatible solid or liquid fillers or diluents or encapsulating compounds may be used as well, which are suitable for administration to a subject. The term “compatible” as used herein means that the constituents of the composition are capable of being mixed with the at least one nucleic acid and, optionally, a plurality of nucleic acids of the composition, in such a manner that no interaction occurs, which would substantially reduce the biological activity or the pharmaceutical effectiveness of the composition under typical use conditions (e.g. intramuscular or intradermal administration). Pharmaceutically acceptable carriers or excipients must have sufficiently high purity and sufficiently low toxicity to make them suitable for administration to a subject to be treated. Compounds which may be used as pharmaceutically acceptable carriers or excipients may be sugars, such as, for example, lactose, glucose, trehalose, mannose, and sucrose; starches, such as, for example, corn starch or potato starch; dextrose; cellulose and its derivatives, such as, for example, sodium carboxymethylcellulose, ethylcellulose, cellulose acetate; powdered tragacanth; malt; gelatin; tallow; solid glidants, such as, for example, stearic acid, magnesium stearate; calcium sulfate; vegetable oils, such as, for example, groundnut oil, cottonseed oil, sesame oil, olive oil, corn oil and oil from theobroma; polyols, such as, for example, polypropylene glycol, glycerol, sorbitol, mannitol and polyethylene glycol; alginic acid.

Pharmaceutical compositions of the present disclosure are suitably sterile and/or pyrogen-free.

### Formulation/Complexation:

In some embodiments, the coding RNA of the pharmaceutical composition is complexed or associated with at least one further compound to obtain a formulated composition. A formulation in that context may have the function of a transfection agent. A formulation in that context may also have the function of protecting the RNA from degradation, e.g. to allow storage, shipment, etc.

In some embodiments, the coding RNA of the pharmaceutical composition is formulated with at least one compound, e.g. peptides, proteins, lipids, polysaccharides, and/or polymers.

In some embodiments, the coding RNA of the pharmaceutical composition is formulated with at least one cationic (cationic or for example ionizable) or polycationic compound (cationic or for example ionizable).

In some embodiments, the coding RNA of the pharmaceutical composition is complexed or associated with or at least partially complexed or partially associated with one or more cationic (cationic or for example ionizable) or polycationic compound.

The term “cationic or polycationic compound” as used herein will be recognized and understood by the person of ordinary skill in the art, and are for example intended to refer to a charged molecule, which is positively charged at a pH value ranging from about 1 to 9, at a pH value ranging from about 3 to 8, at a pH value ranging from about 4 to 8, at a pH value ranging from about 5 to 8, for example at a pH value ranging from about 6 to 8, for example at a pH value ranging from about 7 to 8, for example at a physiological pH, e.g. ranging from about 7.2 to about 7.5. Accordingly, a cationic component, e.g. a cationic peptide, cationic protein, cationic polymer, cationic polysaccharide, cationic lipid may be any positively charged compound or polymer which is positively charged under physiological conditions. A “cationic or polycationic peptide or protein” may contain at least one positively charged amino acid, or more than one positively charged amino acid, e.g. selected from Arg, His, Lys or Orn. Accordingly, “polycationic” components are also within the scope exhibiting more than one positive charge under the given conditions.

In some embodiments, the at least one cationic or polycationic compound is selected from a cationic or polycationic polymer, a cationic or polycationic polysaccharide, a cationic or polycationic lipid, a cationic or polycationic protein, a cationic or polycationic peptide, or any combinations thereof.

In some embodiments, the at least one cationic or polycationic compound is selected from a cationic or polycationic peptide or protein.

In some embodiments, the pharmaceutical composition comprises a coding RNA as defined herein, and a polymeric carrier.

The term “polymeric carrier” as used herein will be recognized and understood by the person of ordinary skill in the art, and are e.g. intended to refer to a compound that facilitates transport and/or complexation of another compound. A polymeric carrier is typically a carrier that is formed of a polymer. A polymeric carrier may be associated to its cargo (e.g. RNA) by covalent or non-covalent interaction. A polymer may be based on different subunits, such as a copolymer. Suitable polymeric carriers in that context may include, for example, polyethylenimine (PEI).

In embodiments, the pharmaceutical composition comprises at least one RNA that is complexed or associated with polymeric carriers and, optionally, with at least one lipid component as described in WO2017212008, WO2017212006, WO2017212007, and WO2017212009. In this context, the disclosures of WO2017212008, WO2017212006, WO2017212007, and WO2017212009 are herewith incorporated by reference. In some embodiments, the lipidoid component of the polymeric carrier may be any one selected from the table of lipidoid structures of published PCT patent application WO2017212009A1 (pages 50-54).

### Formulation in Lipid-Based Carriers:

In some embodiments, the pharmaceutical composition comprises lipid-based carriers.

In the context of the disclosure, the term “lipid-based carriers” encompass lipid based delivery systems for RNA that comprise a lipid component. A lipid-based carrier may additionally comprise other components suitable for encapsulating/incorporating/complexing an RNA including a cationic or polycationic polymer, a cationic or polycationic polysaccharide, a cationic or polycationic protein, a cationic or polycationic peptide, or any combinations thereof.

In the context of the disclosure, a typical “lipid-based carrier” is selected from liposomes, lipid nanoparticles (LNPs), lipoplexes, and/or nanoliposomes. The RNA of the pharmaceutical composition may completely or partially incorporated or encapsulated in a lipid-based carrier, wherein the RNA may be located in the interior space of the lipid-based carrier, within the lipid layer/membrane of the lipid-based carrier, or associated with the exterior surface of the lipid-based carrier. The incorporation of RNA into lipid-based carriers may be referred to as “encapsulation”. A “lipid-based carrier” is not restricted to any particular morphology, and include any morphology generated when e.g. an aggregation reducing lipid and at least one further lipid are combined, e.g. in an aqueous environment in the presence of RNA. For example, an LNP, a liposome, a lipid complex, a lipoplex and the like are within the scope of the term “lipid-based carrier”. Lipid-based carriers can be of different sizes such as, but not limited to, a multilamellar vesicle (MLV) which may be hundreds of nanometers in diameter and may contain a series of concentric bilayers separated by narrow aqueous compartments, a small unicellular vesicle (SUV) which may be smaller than 50 nm in diameter, and a large unilamellar vesicle (LUV) which may be between 50 nm and 500 nm in diameter. Liposomes, a specific type of lipid-based carrier, are characterized as microscopic vesicles having an interior aqua space sequestered from an outer medium by a membrane of one or more bilayers. In a liposome, the at least one RNA is typically located in the interior aqueous space enveloped by some or the entire lipid portion of the liposome. Bilayer membranes of liposomes are typically formed by amphiphilic molecules, such as lipids of synthetic or natural origin that comprise spatially separated hydrophilic and hydrophobic domains. Lipid nanoparticles (LNPs), a specific type of lipid-based carrier, are characterized as microscopic lipid particles having a solid core or partially solid core. Typically, an LNP does not comprise an interior aqua space sequestered from an outer medium by a bilayer. In an LNP, the at least one RNA may be encapsulated or incorporated in the lipid portion of the LNP enveloped by some or the entire lipid portion of the LNP. An LNP may comprise any lipid capable of forming a particle to which the RNA may be attached, or in which the RNA may be encapsulated. For example, said lipid-based carriers are particularly suitable for intramuscular and/or intradermal administration.

In some embodiments, the lipid-based carriers of the pharmaceutical composition are selected from liposomes, lipid nanoparticles, lipoplexes, and/or nanoliposomes.

In one embodiment, the lipid-based carriers of the pharmaceutical composition are lipid nanoparticles (LNPs). In one embodiment, the lipid nanoparticles of the pharmaceutical composition encapsulate the coding RNA of the disclosure.

The term “encapsulated”, e.g. incorporated, complexed, encapsulated, partially encapsulated, associated, partially associated, refers to the essentially stable combination of RNA with one or more lipids into lipid-based carriers (e.g. larger complexes or assemblies) for example without covalent binding of the RNA. The lipid-based carriers—encapsulated RNA may be completely or partially located in the interior of the lipid-based carrier (e.g. the lipid portion and/or an interior space) and/or within the lipid layer/membrane of the lipid-based carriers. The encapsulation of an RNA into lipid-based carriers is also referred to herein as “incorporation” as the RNA is for example contained within the interior of the lipid-based carriers. Without wishing to be bound to theory, the purpose of incorporating or encapsulating RNA into lipid-based carriers may be to protect the RNA from an environment which may contain enzymes, chemicals, or conditions that degrade the RNA. Moreover, incorporating RNA into lipid-based carriers may promote the uptake of the RNA, and hence, may enhance the therapeutic effect of the RNA when administered to a cell or a subject.

In some embodiments, the lipid-based carriers of the pharmaceutical composition comprise at least one or more lipids selected from at least one aggregation-reducing lipid, at least one cationic lipid, at least one neutral lipid or phospholipid, or at least one steroid or steroid analogue, or any combinations thereof.

In one embodiment, the lipid-based carriers of the pharmaceutical composition comprise an aggregation-reducing lipid, a cationic lipid or ionizable lipid, a neutral lipid or phospholipid, and a steroid or steroid analogue.

### Cationic Lipids:

In some embodiments, the lipid-based carriers comprise a cationic or ionizable lipid.

The cationic or ionizable lipid of the lipid-based carriers may be cationisable or ionizable, i.e. it becomes protonated as the pH is lowered below the pK of the ionizable group of the lipid, but is progressively more neutral at higher pH values. At pH values below the pK, the lipid is then able to associate with negatively charged nucleic acids. In certain embodiments, the cationic lipid comprises a zwitterionic lipid that assumes a positive charge on pH decrease.

In some embodiments, the lipid-based carriers comprise a cationic or ionizable lipid that for example carries a net positive charge at physiological pH, for example the cationic or ionizable lipid comprises a tertiary nitrogen group or quaternary nitrogen group. Accordingly, in some embodiments, the lipid-based carriers comprise a cationic or ionizable lipid selected from an amino lipid.

In further embodiments, the lipid formulation comprises cationic or ionizable lipids as defined in Formula I of paragraph [00251] of WO2021222801 or a lipid selected from the disclosure of paragraphs [00260] or [00261] of WO2021222801. In other embodiments, the lipid formulation comprises cationic or ionizable lipids selected from the group consisting of ATX-001 to ATX-132 as disclosed in claim 90 of WO2021183563, for example ATX-0126. The disclosure of WO2021222801 and WO2021183563, especially aforementioned lipids, are incorporated herewith by reference.

In embodiments, cationic or ionizable lipids may be selected from the lipids disclosed in WO2018078053 (i.e. lipids derived from formula I, II, and III of WO2018078053, or lipids as specified in claims 1 to 12 of WO2018078053), the disclosure of WO2018078053 hereby incorporated by reference in its entirety. In that context, lipids disclosed in Table 7 of WO2018078053 (e.g. lipids derived from formula I-1 to I-41) and lipids disclosed in Table 8 of WO2018078053 (e.g. lipids derived from formula II-1 to II-36) may be suitably used in the context of the disclosure. Accordingly, formula I-1 to formula I-41 and formula II-1 to formula II-36 of WO2018078053, and the specific disclosure relating thereto, are herewith incorporated by reference.

In some embodiments, the lipid-based carriers of the pharmaceutical composition comprise a cationic lipid selected or derived from structures III-1 to III-36 of Table 9 of published PCT patent application WO2018078053. Accordingly, formula III-1 to III-36 of WO2018078053, and the specific disclosure relating thereto, are herewith incorporated by reference.

In various embodiments, the lipid-based carriers (e.g. LNPs) of the pharmaceutical composition of the disclosure (e.g. component B) comprise a cationic lipid according to formula (III) or derived from formula (III):

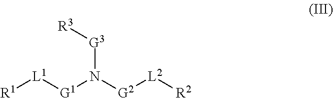

Formula (III) is further defined in that:
one of L1 or L2 is —O(C═O)—, —(C═O)O—, —C(═O)—, —O—, —S(O)x-, —S—S—, —C(═O)S—, SC(=O)—, —NRaC(═O)—, —C(═O)NRa—, —NRaC(═O)NRa—, —OC(═O)NRa— or —NRaC(═O)O—, and the other of L1 or L2 is —O(C═O)—, —(C═O)O—, —C(═O)—, —O—, —S(O)x-, —S—S—, —C(═O)S—, SC(═O)—, —NRaC(═O)—, —C(═O)NRa—, —NRaC(═O)NRa—, —OC(═O)NRa— or —NRaC(═O)O— or a direct bond;
G1 and G2 are each independently unsubstituted C1-C12 alkylene or C1-C12 alkenylene;
G3 is C1-C24 alkylene, C1-C24 alkenylene, C3-C8 cycloalkylene, C3-C8 cycloalkenylene;
Ra is H or C1-C12 alkyl;
R1 and R2 are each independently C6-C24 alkyl or C6-C24 alkenyl;
R3 is H, OR5, CN, C(═O)OR4, OC(═O)R4 or —NR5C(═O)R4;
R4 is C1-C12 alkyl;
R5 is H or C1-C6 alkyl; and
x is 0, 1 or 2.

In one embodiment, the lipid-based carriers comprises a cationic lipid selected or derived from formula III-3:

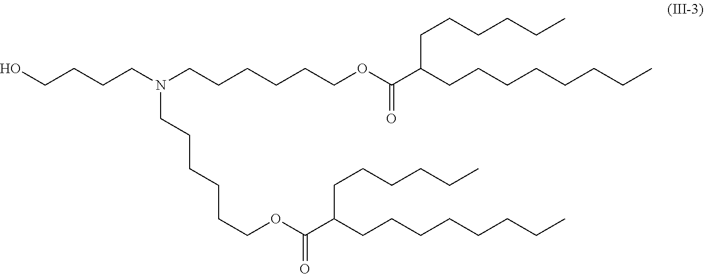 [SMILES: CCCCCCCCC(CCCCCC)C(=O)OCCCCCCN(CCCCO)CCCCCCOC(=O)C(CCCCCC)CCCCCCCC]

The lipid of formula III-3 as suitably used herein has the chemical term ((4-hydroxybutyl)azanediyl)bis(hexane-6,1-diyl)bis(2-hexyldecanoate), also referred to as ALC-0315 i.e. CAS Number 2036272-55-4.

Further suitable cationic lipids may be selected or derived from cationic lipids according to PCT claims 1 to 14 of published patent application WO2021123332, or table 1 of WO2021123332, the disclosure relating to claims 1 to 14 or table 1 of WO2021123332 herewith incorporated by reference.

Accordingly, suitable cationic lipids may be selected or derived from cationic lipids according to Compound 1 to Compound 27 (C1-C27) of Table 1 of WO2021123332.

In other embodiments, the lipid-based carriers (e.g. LNPs) of the pharmaceutical composition comprise a cationic lipid selected or derived from (COATSOME® SS-EC)SS-33/4PE-15 (see C23 in Table 1 of WO2021123332).

In other embodiments, the lipid-based carriers (e.g. LNPs) of the pharmaceutical composition comprise a cationic lipid selected or derived from HEXA-C5DE-PipSS (see C2 in Table 1 of WO2021123332). In some embodiments, the lipid-based carriers (e.g. LNPs) of the pharmaceutical composition comprise a cationic lipid selected or derived from compound C26 as disclosed in Table 1 of WO2021123332:

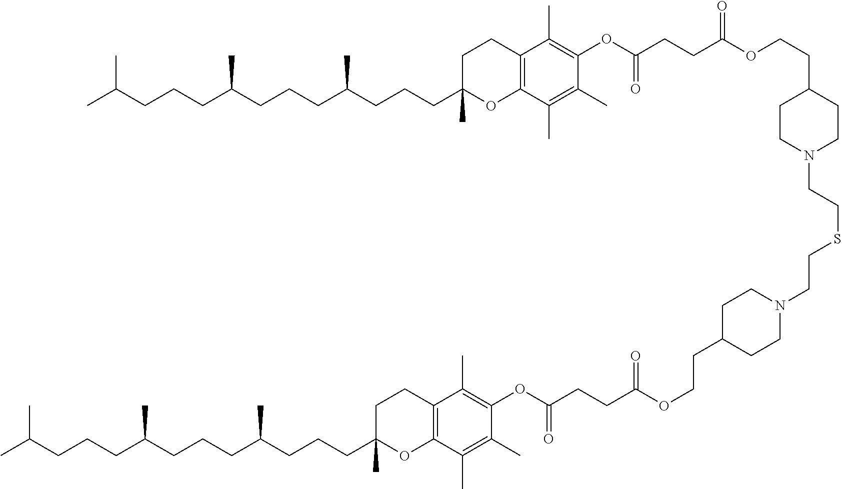 [SMILES: Cc1c(C)c2c(c(C)c1OC(=O)CCC(=O)OCCC1CCN(CCSCCN3CCC(CCOC(=O)CCC(=O)Oc4c(C)c(C)c5c(c4C)CC[C@@](C)(CCC[C@H](C)CCC[C@H](C)CCCC(C)C)O5)CC3)CC1)CC[C@@](C)(CCC[C@H](C)CCC[C@H](C)CCCC(C)C)O2]

In other embodiments, the lipid-based carriers (e.g. LNPs) of the pharmaceutical composition comprise a cationic lipid selected or derived from 9-Heptadecanyl 8-{(2-hydroxyethyl)[6-oxo-6-(undecyloxy)hexyl]amino}octanoate, also referred to as SM-102. Other lipid-based carriers (e.g. LNPs) of the pharmaceutical composition comprise a squaramide ionizable amino lipid, for example a cationic lipid selected from the group consisting of formulas (M1) and (M2):

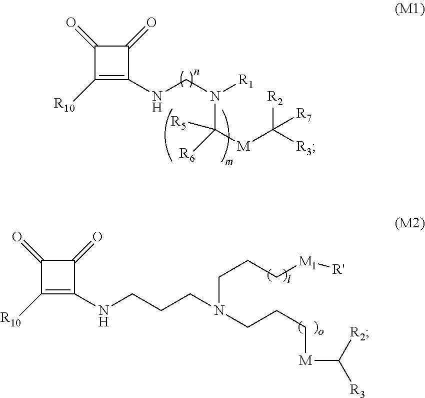

wherein the substituents (e.g. R₁, R₂, R₃, R₅, R₆, R₇, R₁₀, M, M₁, m, n, o, l) are defined in claims 1 to 13 of U.S. Ser. No. 10/392,341 B2; U.S. Ser. No. 10/392,341B2 being incorporated herein in its entirety.

Accordingly, in some embodiments, the lipid-based carriers (e.g. LNPs) of the pharmaceutical composition comprise a cationic lipid selected or derived from above mentioned ALC-0315, SM-102, SS-33/4PE-15, HEXA-C5DE-PipSS, or compound 026 (see 026 in Table 1 of WO2021123332).

In one embodiment, the lipid-based carriers, for example the LNPs, of the pharmaceutical composition comprise a cationic lipid selected or derived from ALC-0315.

In some embodiments, the lipid-based carriers of the disclosure comprise two or more (different) cationic lipids as defined herein.

In certain embodiments, the cationic lipid as defined herein, for example cationic lipid ALC-0315, is present in the lipid-based carriers in an amount from about 30 mol % to about 95 mol %, relative to the total lipid content of the lipid-based carriers. If more than one cationic lipid is incorporated within the lipid-based carriers, such percentages apply to the combined cationic lipids.

In embodiments, the cationic lipid is present in the lipid-based carriers in an amount from about 30 mol % to about 70 mol %. In one embodiment, the cationic lipid is present in the lipid-based carriers in an amount from about 40 mol % to about 60 mol %, such as about 40, 41, 42, 43, 44, 45, 46, 47, 48, 49, 50, 51, 52, 53, 54, 55, 56, 57, 58, 59 or 60 mol %, respectively. In embodiments, the cationic lipid is present in the lipid-based carriers in an amount from about 47 mol % to about 48 mol %, such as about 47.0, 47.1, 47.2, 47.3, 47.4, 47.5, 47.6, 47.7, 47.8, 47.9, 50.0 mol %, respectively, wherein 47.4 mol % are particularly suitable. In other embodiments, the cationic lipid is present in the lipid-based carriers in an amount from about 55 mol % to about 65 mol %, such as about 55, 56, 57, 58, 59, 60, 61, 62, 63, 64 or 65 mol %, respectively, wherein 59 mol % are particularly suitable.

In some embodiments, the cationic lipid is present in a ratio of from about 20 mol % to about 70 mol % or 75 mol % or from about 45 mol % to about 65 mol % or about 20, 25, 30, 35, 40, 45, 50, 55, 60, 65, or about 70 mol % of the total lipid present in the lipid-based carriers. In further embodiments, the LNPs comprise from about 25% to about 75% on a molar basis of cationic lipid, e.g., from about 20 to about 70%, from about 35 to about 65%, from about 45 to about 65%, about 60%, about 57.5%, about 57.1%, about 50% or about 40% on a molar basis (based upon 100% total moles of lipid in the lipid nanoparticle).

In some embodiments, the ratio of cationic lipid to RNA is from about 3 to about 15, such as from about 5 to about 13 or from about 7 to about 11.

### Aggregation Reducing Lipids:

The term “aggregation reducing lipid” refers to a molecule comprising both a lipid portion and a moiety suitable of reducing or preventing aggregation of the lipid-based carriers. Under storage conditions or during formulation, the lipid-based carriers may undergo charge-induced aggregation, a condition which can be undesirable for the stability of the lipid-based carriers. Therefore, it can be desirable to include a lipid compound which can reduce aggregation, for example by sterically stabilizing the lipid-based carriers. Such a steric stabilization may occur when a compound having a sterically bulky but uncharged moiety that shields or screens the charged portions of a lipid-based carriers from close approach to other lipid-based carriers in the composition. In the context of the disclosure, stabilization of the lipid-based carriers is achieved by including lipids which may comprise a lipid bearing a sterically bulky group which, after formation of the lipid-based carrier, is for example located on the exterior of the lipid-based carrier. Suitable aggregation reducing groups include hydrophilic groups, e.g. monosialoganglioside GM1, polyamide oligomers (PAO), or certain polymers, such as poly(oxyalkylenes), e.g. poly(ethylene glycol) or poly(propylene glycol).

Lipids comprising a polymer as aggregation reducing group are herein referred to as “polymer conjugated lipid”.

The term “polymer conjugated lipid” refers to a molecule comprising both a lipid portion and a polymer portion, wherein the polymer is suitable of reducing or preventing aggregation of lipid-based carriers comprising the RNA. A polymer has to be understood as a substance or material consisting of very large molecules, or macromolecules, composed of many repeating subunits. A suitable polymer in the context of the disclosure may be a hydrophilic polymer. An example of a polymer conjugated lipid is a PEGylated or PEG-conjugated lipid.

In one embodiment, the lipid-based carriers of the pharmaceutical composition comprise an aggregation reducing lipid selected from a polymer conjugated lipid.

In some embodiments, the polymer conjugated lipid is a PEG-conjugated lipid (or PEGylated lipid, PEG lipid).

The average molecular weight of the PEG moiety in the PEG-conjugated lipid for example ranges from about 500 to about 8,000 Daltons (e.g., from about 1,000 to about 4,000 Daltons). In one embodiment, the average molecular weight of the PEG moiety is about 2,000 Daltons.

In some embodiments, the PEG-conjugated lipid is selected from PEG-modified phosphatidylethanolamine, PEG-modified phosphatidic acid, PEG-modified ceramides (e.g. PEG-CerC14 or PEG-CerC20), PEG-modified dialkylamines, PEG-modified diacylglycerols, PEG-modified dialkylglycerols. Representative polyethylene glycol-lipids include PEG-c-DOMG, PEG-c-DMA, and PEG-s-DMG. In one embodiment, the polyethylene glycol-lipid is N-[(methoxy poly(ethylene glycol)2000)carbamyl]-1,2-dimyristyloxlpropyl-3-amine (PEG-c-DMA). In one embodiment, the polyethylene glycol-lipid is DMG-PEG 2000. In one embodiment, the polyethylene glycol-lipid is PEG-c-DOMG). In other embodiments, the LNPs comprise a PEGylated diacylglycerol (PEG-DAG) such as 1-(monomethoxy-polyethyleneglycol)-2,3-dimyristoylglycerol (PEG-DMG), a PEGylated phosphatidylethanoloamine (PEG-PE), a PEG succinate diacylglycerol (PEG-S-DAG) such as 4-O-(2′,3′-di(tetradecanoyloxy)propyl-1-O-(ω-methoxy(polyethoxy)ethyl)butanedioate (PEG-S-DMG), a PEGylated ceramide (PEG-cer), or a PEG dialkoxypropylcarbamate such as w-methoxy(polyethoxy)ethyl-N-(2,3-di(tetradecanoxy)propyl)carbamate or 2,3-di(tetradecanoxy)propyl-N-(w-methoxy(polyethoxy)ethyl)carbamate.

In some embodiments, the polymer conjugated lipid, e.g. the PEG-conjugated lipid, is selected or derived from 1,2-dimyristoyl-rac-glycero-3-methoxypolyethylene glycol-2000 (PEG2000 DMG or DMG-PEG 2000). As used in the art, “DMG-PEG 2000” is typically considered a mixture of 1,2-DMG PEG 2000 and 1,3-DMG PEG 2000 in ˜97:3 ratio.

In other embodiments, the polymer conjugated lipid, e.g. the PEG-conjugated lipid, is selected or derived from C10-PEG2K, or Cer8-PEG2K.

In one embodiment, the polymer conjugated lipid, e.g. the PEG-conjugated lipid is selected or derived from formula (IVa):

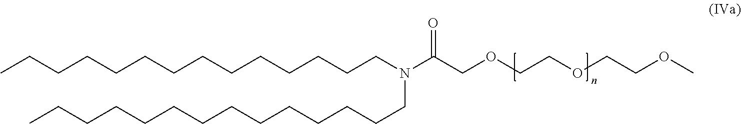

for example wherein n has a mean value ranging from 30 to 60, such as about 30±2, 32±2, 34±2, 36±2, 38±2, 40±2, 42±2, 44±2, 46±2, 48±2, 50±2, 52±2, 54±2, 56±2, 58±2, or 60±2. In one embodiment n is about 49. In another embodiment n is 45. In further embodiments said PEG lipid is of formula (IVa), wherein n is an integer selected such that the average molecular weight of the PEG lipid is about 2000 g/mol to about 3000 g/mol or about 2300 g/mol to about 2700 g/mol. In another embodiment said PEG lipid is of formula (IVa), wherein n is an integer selected such that the average molecular weight of the PEG lipid is about 2000 g/mol.

The PEG-conjugated lipid of formula Va as suitably used herein has the chemical 2[(polyethylene glycol)-2000]-N,N-ditetradecylacetamide, also referred to as ALC-0159.

Accordingly, in some embodiments, the aggregation reducing lipid is a PEG-conjugated lipid selected or derived from DMG-PEG 2000, C10-PEG2K, Cer8-PEG2K, or ALC-0159.

In one embodiment, the lipid-based carriers, for example the LNPs of the pharmaceutical composition comprise an aggregation reducing lipid selected or derived from ALC-0159.

In some embodiments, the lipid-based carriers of the pharmaceutical composition comprise an aggregation reducing lipid, wherein the aggregation reducing lipid is not a PEG-conjugated lipid.

In some embodiments, lipid-based carriers include less than about 3 mol %, 2 mol %, or 1 mol % of aggregation reducing lipid, based on the total moles of lipid in the lipid-based carrier. In further embodiments, lipid-based carriers comprise from about 0.1% to about 10% of the aggregation reducing lipid on a molar basis, e.g. about 0.5% to about 10%, about 0.5% to about 5%, about 10%, about 5%, about 4%, about 3%, about 2%, about 1.5%, about 1%, about 0.5%, or about 0.3% on a molar basis (based on 100% total moles of lipids in the lipid-based carrier). In other embodiments, lipid-based carriers comprise from about 1.0% to about 2.0% of the aggregation reducing lipid on a molar basis, e.g. about 1.2% to about 1.9%, about 1.2% to about 1.8%, about 1.3% to about 1.8%, about 1.4% to about 1.8%, about 1.5% to about 1.8%, about 1.6% to about 1.8%, in particular about 1.4%, about 1.5%, about 1.6%, about 1.7%, about 1.8%, about 1.9%, for example 1.7% (based on 100% total moles of lipids in the lipid-based carrier). In other embodiments, lipid-based carriers comprise about 2.5%, 3%, 3.5%, 4%, 4.5% or 5%, for example 2.5% of the aggregation reducing lipid on a molar basis (based on 100% total moles of lipids in the lipid-based carrier). In various embodiments, the molar ratio of the cationic lipid to the aggregation reducing lipid ranges from about 100:1 to about 25:1.

### Neutral Lipids:

In some embodiments, the lipid-based carriers (e.g. LNPs) comprise a neutral lipid or phospholipid.

The term “neutral lipid” refers to any one of a number of lipid species that exist in either an uncharged or neutral zwitterionic form at physiological pH. Suitable neutral lipids include diacylphosphatidylcholines, diacylphosphatidylethanolamines, ceramides, sphingomyelins, dihydrosphingomyelins, cephalins, and cerebrosides. The selection of neutral lipids for use in the particles described herein is generally guided by consideration of, e.g., lipid particle size and stability of the lipid particle in the bloodstream. For example, the neutral lipid is a lipid having two acyl groups (e.g. diacylphosphatidylcholine and diacylphosphatidylethanolamine). In one embodiment, the neutral lipids contain saturated fatty acids with carbon chain lengths in the range of C10 to C20. In another embodiment, neutral lipids with mono or diunsaturated fatty acids with carbon chain lengths in the range of C10 to C20 are used. Additionally, neutral lipids having mixtures of saturated and unsaturated fatty acid chains can be used.

In some embodiments, the lipid-based carriers comprises one or more neutral lipids, wherein the neutral lipid is selected from the group comprising distearoylphosphatidylcholine (DSPC), dioleoylphosphatidylcholine (DOPC), dipalmitoylphosphatidylcholine (DPPC), dioleoylphosphatidylglycerol (DOPG), dipalmitoylphosphatidylglycerol (DPPG), dioleoyl-phosphatidylethanolamine (DOPE), palmitoyloleoylphosphatidylcholine (POPC), palmitoyloleoyl-phosphatidylethanolamine (POPE) and dioleoyl-phosphatidylethanolamine 4-(N-maleimidomethyl)-cyclohexane-1carboxylate (DOPE-mal), dipalmitoyl phosphatidyl ethanolamine (DPPE), dimyristoylphosphoethanolamine (DMPE), distearoyl-phosphatidylethanolamine (DSPE), 16-O-monomethyl PE, 16-O-dimethyl PE, 18-1-trans PE, 1-stearioyl-2-oleoylphosphatidyethanol amine (SOPE), and 1,2-dielaidoyl-sn-glycero-3-phophoethanolamine (transDOPE), 1,2-diphytanoyl-sn-glycero-3-phosphoethanolamine (DPhyPE), or mixtures thereof.

In some embodiments, the neutral lipid of the lipid-based carriers (e.g. LNPs) of the pharmaceutical composition is selected or derived from 1,2-diheptanoyl-sn-glycero-3-phosphocholine (DHPC).

In other embodiments, the neutral lipid of the lipid-based carriers (e.g. LNPs) of the pharmaceutical composition is selected or derived from 1,2-diphytanoyl-sn-glycero-3-phosphoethanolamine (DPhyPE).

Accordingly, in some embodiments, the lipid-based carriers (e.g. LNPs) of the pharmaceutical composition comprise a neutral lipid selected or derived from DSPC, DHPC, or DPhyPE.

In some embodiments, the lipid-based carriers, for example the LNPs of the pharmaceutical composition comprise a neutral lipid selected or derived from 1,2-distearoyl-sn-glycero-3-phosphocholine (DSPC).

In various embodiments, the molar ratio of the cationic lipid to the neutral lipid in the lipid-based carriers ranges from about 2:1 to about 8:1.

The neutral lipid is for example from about 5 mol % to about 90 mol %, about 5 mol % to about 10 mol %, about 5, 10, 15, 20, 25, 30, 35, 40, 45, 50, 55, 60, 65, 70, 75, 80, 85, or about 90 mol % of the total lipid present in the lipid-based carrier. In one embodiment, the lipid-based carrier includes from about 0% to about 15% or 45% on a molar basis of neutral lipid, e.g. from about 3% to about 12% or from about 5% to about 10%. For instance, the lipid-based carrier may include about 15%, about 10%, about 7.5%, or about 7.1% of neutral lipid on a molar basis (based upon 100% total moles of lipid in the lipid-based carrier).

### Steroids, Steroid Analogues or Sterols:

In some embodiments, the lipid-based carriers of the pharmaceutical composition comprise a steroid, steroid analogue or sterol.

Suitably, the steroid, steroid analogue or sterol may be derived or selected from cholesterol, cholesteryl hemisuccinate (CHEMS) and a derivate thereof. In other embodiments, the lipid-based carriers of the pharmaceutical composition comprise a steroid, steroid analogue or sterol derived from a phytosterol (e.g., a sitosterol, such as beta-sitosterol), for example from a compound having the structure of Formula I as disclosed in claim 1 of WO2020061332; the disclosure of WO2020061332, especially the disclosure of Formula I and phytosterols being incorporated by reference herewith. In a further embodiment, the steroid is an imidazole cholesterol ester or “ICE” as disclosed in paragraphs [0320] and [0339]-[0340] of WO2019226925; WO2019226925 being incorporated herein by reference in its entirety.

In some embodiments, the lipid-based carriers of the pharmaceutical composition comprise a steroid, steroid analogue or sterol, which is selected or derived from cholesterol or comprise cholesterol.

The molar ratio of the cationic lipid to cholesterol in the lipid-based carriers may be in the range from about 2:1 to about 1:1. In some embodiments, the cholesterol may be PEGylated.

In some embodiments, the lipid-based carrier comprises about 10 mol % to about 60 mol % or about 25 mol % to about 40 mol % sterol (based on 100% total moles of lipids in the lipid-based carrier). In one embodiment, the sterol is about 10, 15, 20, 25, 30, 35, 40, 45, 50, 55, or about 60 mol % of the total lipid present in the lipid-based carrier. In another embodiment, the lipid-based carriers include from about 5% to about 50% on a molar basis of the sterol, e.g., about 15% to about 45%, about 20% to about 40%, about 48%, about 40%, about 38.5%, about 35%, about 34.4%, about 31.5% or about 30% on a molar basis (based upon 100% total moles of lipid in the lipid-based carrier). In some embodiments, the lipid-based carrier comprises about 28%, about 29% or about 30% sterol (based on 100% total moles of lipids in the lipid-based carrier). In some embodiments, the lipid-based carrier comprises about 40.9% sterol (based on 100% total moles of lipids in the lipid-based carrier).

References to other suitable cationic or ionizable, neutral, steroid/sterol or aggregation reducing lipids:

Other suitable cationic or ionizable, neutral, steroid/sterol or aggregation reducing lipids are disclosed in WO2010053572, WO2011068810, WO2012170889, WO2012170930, WO2013052523, WO2013090648, WO2013149140, WO2013149141, WO2013151663, WO2013151664, WO2013151665, WO2013151666, WO2013151667, WO2013151668, WO2013151669, WO2013151670, WO2013151671, WO2013151672, WO2013151736, WO2013185069, WO2014081507, WO2014089486, WO2014093924, WO2014144196, WO2014152211, WO2014152774, WO2014152940, WO2014159813, WO2014164253, WO2015061461, WO2015061467, WO2015061500, WO2015074085, WO2015105926, WO2015148247, WO2015164674, WO2015184256, WO2015199952, WO2015200465, WO2016004318, WO2016022914, WO2016036902, WO2016081029, WO2016118724, WO2016118725, WO2016176330, WO2017004143, WO2017019935, WO2017023817, WO2017031232, WO2017049074, WO2017049245, WO2017070601, WO2017070613, WO2017070616, WO2017070618, WO2017070620, WO2017070622, WO2017070623, WO2017070624, WO2017070626, WO2017075038, WO2017075531, WO2017099823, WO2017106799, WO2017112865, WO2017117528, WO2017117530, WO2017180917, WO2017201325, WO2017201340, WO2017201350, WO2017201352, WO2017218704, WO2017223135, WO2018013525, WO2018081480, WO2018081638, WO2018089540, WO2018089790, WO2018089801, WO2018089851, WO2018107026, WO2018118102, WO2018119163, WO2018157009, WO2018165257, WO2018170245, WO2018170306, WO2018170322, WO2018170336, WO2018183901, WO2018187590, WO2018191657, WO2018191719, WO2018200943, WO2018231709, WO2018231990, WO2018232120, WO2018232357, WO2019036000, WO2019036008, WO2019036028, WO2019036030, WO2019040590, WO2019089818, WO2019089828, WO2019140102, WO2019152557, WO2019152802, WO2019191780, WO2019222277, WO2019222424, WO2019226650, WO2019226925, WO2019232095, WO2019232097, WO2019232103, WO2019232208, WO2020061284, WO2020061295, WO2020061332, WO2020061367, WO2020081938, WO2020097376, WO2020097379, WO2020097384, WO2020102172, WO2020106903, WO2020146805, WO2020214946, WO2020219427, WO2020227085, WO2020232276, WO2020243540, WO2020257611, WO2020257716, WO2021007278, WO2021016430, WO2021022173, WO2021026358, WO2021030701, WO2021046260, WO2021050986, WO2021055833, WO2021055835, WO2021055849, WO2021127394, WO2021127641, WO2021202694, WO2021231697, WO2021231901, WO2008103276, WO2009086558, WO2009127060, WO2010048536, WO2010054406, WO2010080724, WO2010088537, WO2010129709, WO201021865, WO2011022460, WO2011043913, WO2011090965, WO2011149733, WO2011153120, WO2011153493, WO2012040184, WO2012044638, WO2012054365, WO2012061259, WO2013063468, WO2013086354, WO2013086373, U.S. Pat. No. 7,893,302B2, U.S. Pat. No. 7,404,969B2, U.S. Pat. No. 8,158,601B2, U.S. Pat. No. 8,283,333B2, U.S. Pat. No. 8,466,122B2, U.S. Pat. No. 8,569,256B2, US20100036115, US20110256175, US20120202871, US20120027803, US20120128760, US20130064894, US20130129785, US20130150625, US20130178541, US20130225836, and US20140039032; the disclosures specifically relating to cationic or ionizable, neutral, sterol or aggregation reducing lipids suitable for lipid-based carriers of the foregoing publications are incorporated herewith by reference.

For example, suitable cationic lipids or cationisable or ionizable lipids include, but are not limited to, DSDMA, N,N-dioleyl-N,N-dimethylammonium chloride (DODAC), N,N-distearyl-N,N-dimethylammonium bromide (DDAB), 1,2-dioleoyltrimethyl ammonium propane chloride (DOTAP) (also known as N-(2,3-dioleoyloxy)propyl)-N,N,N-trimethylammonium chloride and 1,2-Dioleyloxy-3-trimethylaminopropane chloride salt), N-(1-(2,3-dioleyloxy)propyl)-N,N,N-trimethylammonium chloride (DOTMA), N,N-dimethyl-2,3-dioleyloxy)propylamine (DODMA), ckk-E12 (WO2015200465), 1,2-DiLinoleyloxy-N,N-dimethylaminopropane (DLinDMA), 1,2-Dilinolenyloxy-N,N-dimethylaminopropane (DLenDMA), 1,2-di-γ-linolenyloxy-N,N-dimethylaminopropane (γ-DLenDMA), 98N12-5, 1,2-Dilinoleylcarbamoyloxy-3-dimethylaminopropane (DLin-C-DAP), ICE (Imidazol-based), HGT5000, HGT5001, DMDMA, CLinDMA, CpLinDMA, DMOBA, DOcarbDAP, DLincarbDAP, DLinCdAP, KLin-K-DMA, DLin-K-XTC2-DMA, XTC (2,2-Dilinoleyl-4-dimethylaminoethyl-[1,3]-dioxolane) HGT4003, 1,2-Dilinoleoyl-3-trimethylaminopropane chloride salt (DLin-TAP.Cl), 1,2-Dilinoleyloxo-3-(2-N,N-dimethylamino)ethoxypropane (DLin-EG-DM A), 2,2-Dilinoleyl-4-dimethylaminomethyl-[1,3]-dioxolane (DLin-K-DMA), (6Z,9Z,28Z,31Z)-heptatriaconta-6,9,28,31-tetraen-19-yl-4-(dimethylamino)butanoate (MC3, US20100324120), ALNY-100 ((3aR,5s,6aS)-N,N-dimethyl-2,2-di((9Z,12Z)-octadeca-9,12-dienyl)tetrahydro-3aH-cyclopenta[d][1,3]dioxol-5-amine)), NC98-5 (4,7,13-tris(3-oxo-3-(undecylamino)propyl)-N,N 16-diundecyl-4,7,10,13-tetraazahexadecane-1,16-diamide), (6Z,9Z,28Z,31Z)-heptatriaconta-6,9,28,31-tetraen-19-yl 4-(dimethylamino) butanoate (DLin-M-C3-DMA), 3-((6Z,9Z,28Z,31Z)-heptatriaconta-6,9,28,31-tetraen-19-yloxy)-N,N-dimethylpropan-1-amine (MC3 Ether), 4-((6Z,9Z,28Z,31Z)-heptatriaconta-6,9,28,31-tetraen-19-yloxy)-N,N-dimethylbutan-1-amine (MC4 Ether), LIPOFECTIN® (commercially available cationic liposomes comprising DOTMA and 1,2-dioleoyl-sn-3phosphoethanolamine (DOPE), from GIBCO/BRL, Grand Island, N.Y.); LIPOFECTAMINE® (commercially available cationic liposomes comprising N-(1-(2,3dioleyloxy)propyl)-N-(2-(sperminecarboxamido)ethyl)-N,N-dimethylammonium trifluoroacetate (DOSPA) and (DOPE), from GIBCO/BRL); and TRANSFECTAM® (commercially available cationic lipids comprising dioctadecylamidoglycyl carboxyspermine (DOGS) in ethanol from Promega Corp., Madison, Wis.) or any combination of any of the foregoing. Further suitable cationic or ionizable lipids include those described in international patent publications WO2010053572 (and particularly, 1,1′-(2-(4-(2-((2-(bis(2-hydroxydodecyl)amino)ethyl)(2-hydroxydodecyl)amino)ethyl)piperazin-1-yl)ethylazane-diyl)didodecan-2-ol (C12-200) described at paragraph [00225] of WO2010053572) and WO2012170930, both of which are incorporated herein by reference, HGT4003, HGT5000, HGTS001, HGT5001, HGT5002 (see US2015140070), 1,2-dilinoleyoxy-3-(dimethylamino)acetoxypropane (DLin-DAC), 1,2-dilinoleyoxy-3morpholinopropane (DLin-MA), 1,2-dilinoleoyl-3-dimethylaminopropane (DLinDAP), 1,2-dilinoleylthio-3-dimethylaminopropane (DLin-S-DMA), 1-linoleoyl-2-linoleyloxy-3dimethylaminopropane (DLin-2-DMAP), 1,2-dilinoleyloxy-3-trimethylaminopropane chloride salt (DLin-TMA.Cl), 1,2-dilinoleyloxy-3-(N-methylpiperazino)propane (DLin-MPZ), 3-(N,N-dilinoleylamino)-1,2-propanediol (DLinAP), 3-(N,N-dioleylamino)-1,2-propanediol (DOAP), 1,2-dilinoleyloxo-3-(2-N,N-dimethylamino)ethoxypropane (DLin-EG-DMA), 2,2-dilinoleyl-4-(2-dimethylaminoethyl)-[1,3]-dioxolane (DLin-KC2-DMA, WO2010042877); dilinoleyl-methyl-4-dimethylaminobutyrate (DLin-MC3-DMA).

### Lipid-Based Carrier Compositions:

In some embodiments, the lipid-based carriers of the pharmaceutical composition, for example the LNPs, comprise a coding RNA as defined in the first aspect, a cationic lipid as defined herein, an aggregation reducing lipid as defined herein, optionally, a neutral lipid as defined herein, and, optionally, a steroid or steroid analogue as defined herein.

In some embodiments, the lipid-based carriers comprising a coding RNA of the first aspect comprise
(i) at least one cationic lipid or ionizable lipid, for example as defined herein;
(ii) at least one neutral lipid or phospholipid, for example as defined herein;
(iii) at least one steroid or steroid analogue, for example as defined herein; and
(iv) at least one aggregation reducing lipid, for example as defined herein.

In some embodiments, the lipid-based carriers comprising a coding RNA of the first aspect comprise
(i) at least one cationic lipid selected or derived from ALC-0315, SM-102, SS-33/4PE-15, HEXA-C5DE-PipSS or compound C26 (see C26 in Table 1 of WO2021123332);
(ii) at least one neutral lipid selected or derived from DSPC, DHPC, or DPhyPE;
(iii) at least one steroid or steroid analogue selected or derived from cholesterol; and
(iv) at least one aggregation reducing lipid selected or derived from DMG-PEG 2000, C10-PEG2K, Cer8-PEG2K, or ALC-0159; and
wherein the lipid-based carriers encapsulate the RNA.

In some embodiments, the cationic lipids (as defined herein), neutral lipid (as defined herein), steroid or steroid analogue (as defined herein), and/or aggregation reducing lipid (as defined herein) may be combined at various relative ratios.

In some embodiments, the lipid-based carriers comprise (i) to (iv) in a molar ratio of about 20-60% cationic lipid or ionizable lipid, about 5-25% neutral lipid, about 25-55% steroid or steroid analogue, and about 0.5-15% aggregation reducing lipid e.g. polymer conjugated lipid, for example wherein the lipid-based carriers encapsulate the RNA.

For example, the ratio of cationic lipid or ionizable lipid to neutral lipid to steroid or steroid analogue to aggregation reducing lipid may be between about 30-60:20-35:20-30:1-15, or at a ratio of about 40:30:25:5, 50:25:20:5, 50:20:25:5, 50:27:20:3, 40:30:20:10, 40:32:20:8, 40:32:25:3 or 40:33:25:2, respectively.

In some embodiments, the lipid-based carriers, for example the LNPs comprising a coding RNA of the first aspect comprise
(i) at least one cationic lipid selected from SM-102;
(ii) at least one neutral lipid selected from DSPC;
(iii) at least one steroid or steroid analogue selected from cholesterol; and
(iv) at least one aggregation reducing lipid selected from DMG-PEG 2000; and
wherein the lipid-based carriers encapsulate the RNA, for example wherein i) to (iv) are n a weight ratio of about 50% cationic lipid, about 10% neutral lipid, about 38.5% steroid or steroid analogue, and about 1.5% aggregation reducing lipid,
for example wherein the lipid-based carriers encapsulate the RNA.

In some embodiments, the lipid-based carriers, for example the LNPs comprising a coding RNA of the first aspect comprise
(i) at least one cationic lipid selected from SM-102;
(ii) at least one neutral lipid selected from DSPC;
(iii) at least one steroid or steroid analogue selected from cholesterol; and
(iv) at least one aggregation reducing lipid selected from DMG-PEG 2000; and
wherein the lipid-based carriers encapsulate the RNA, for example wherein i) to (iv) are n a weight ratio of about 48.5% cationic lipid, about 11.1% neutral lipid, about 38.9% steroid or steroid analogue, and about 1.5% aggregation reducing lipid,
for example wherein the lipid-based carriers encapsulate the RNA. A suitable N/P ratio for this formulation is about 4.85 (lipid to RNA mol ratio).

In some embodiments, the lipid-based carriers, for example the LNPs comprising a coding RNA of the first aspect comprise
(i) at least one cationic lipid selected from SS-33/4PE-15, HEXA-C5DE-PipSS or or compound C26 (see C26 in Table 1 of WO2021123332);
(ii) at least one neutral lipid selected from DPhyPE;
(iii) at least one steroid or steroid analogue selected from cholesterol; and
(iv) at least one aggregation reducing lipid selected from DMG-PEG 2000; and
wherein the lipid-based carriers encapsulate the RNA. Such LNPs are herein referred to as GN-LNPs.

In one embodiment, the lipid-based carriers, for example the LNPs comprising a coding RNA of the first aspect comprise
(i) at least one cationic lipid selected from ALC-0315;
(ii) at least one neutral lipid selected from DSPC;
(iii) at least one steroid or steroid analogue selected from cholesterol; and
(iv) at least one aggregation reducing lipid selected from ALC-0159.

In one embodiment, the lipid-based carriers, preferably the LNPs comprising a coding RNA of the first aspect comprise
(i) at least one cationic lipid selected from ALC-0315;
(ii) at least one neutral lipid selected from DSPC;
(iii) at least one steroid or steroid analogue selected from cholesterol; and
(iv) at least one aggregation reducing lipid selected from ALC-0159; and
wherein the lipid-based carriers encapsulate the RNA, for example wherein i) to (iv) are in a molar ratio of about 47.4% cationic lipid, about 10% neutral lipid, about 40.9% steroid or steroid analogue, and about 1.7% aggregation reducing lipid, for example wherein the lipid-based carriers encapsulate the RNA. Such LNPs are herein referred to as 315-LNPs.

In some embodiments, the pharmaceutical composition comprises lipid nanoparticles (LNPs) which have a molar ratio of approximately 50:10:38.5:1.5, for example 47.5:10:40.8:1.7 or for example 47.4:10:40.9:1.7 (i.e. proportion (mol %) of cationic lipid (for example above mentioned lipid 111-3 (ALC-0315)), DSPC, cholesterol and PEG-lipid (for example above mentioned PEG-lipid of formula (IVa) with n=49, for example above mentioned PEG-lipid of formula (IVa) with n=45 (ALC-0159)); solubilized in ethanol).

In one embodiment, the lipid-based carriers, for example the LNPs comprising a coding RNA of the first aspect comprise
(i) at least one cationic lipid selected from ALC-0315;
(ii) at least one neutral lipid selected from DSPC;
(iii) at least one steroid or steroid analogue selected from cholesterol; and
(iv) at least one aggregation reducing lipid selected from ALC-0159; and
wherein the lipid-based carriers encapsulate the RNA, for example wherein i) to (iv) are in a molar ratio of about 47.4% cationic lipid, about 10% neutral lipid, about 40.9% steroid or steroid analogue, and about 1.7% aggregation reducing lipid, for example wherein the lipid-based carriers encapsulate the RNA, wherein the RNA comprises or consists of an RNA sequence identical or at least 70%, 80%, 90%, 91%, 92%, 93%, 94%, 95%, 96%, 97%, 98%, or 99% identical to any one of SEQ ID NOs: 673-695, 698-720, 723-745, 748-770, 773-795, 798-820, 823-845, 848-870, 873-895, 898-920, 923-945, 948-970, 973-995, 998-1020, 1023-1045, 1048-1070, 1073-1095, 1098-1120, 1123-1145, 1148-1170, 1173-1195, 1198-1220, 1223-1245, 1248-1270, or a fragment or a variant thereof. In such embodiments, the RNA is for example an mRNA that comprises a cap1 structure and an RNA sequence that is not chemically modified (e.g. consisting of non-modified ribonucleotides).

mRNA sequences in that context are for example SEQ ID NOs: 829, 679, or a fragment or a variant of any of these. Other mRNA sequences in that context are SEQ ID NOs: 834, 684, or a fragment or a variant of any of these. Other mRNA sequences in that context are SEQ ID NOs: 833, 683, or a fragment or a variant of any of these.

In some embodiments, the lipid-based carriers, for example the LNPs comprising a coding RNA of the first aspect comprise
(i) at least one cationic lipid selected from ALC-0315;
(ii) at least one neutral lipid selected from DSPC;
(iii) at least one steroid or steroid analogue selected from cholesterol; and
(iv) at least one aggregation reducing lipid selected from ALC-0159; and
wherein the lipid-based carriers encapsulate the RNA, for example wherein i) to (iv) are in a molar ratio of about 47.4% cationic lipid, about 10% neutral lipid, about 40.9% steroid or steroid analogue, and about 1.7% aggregation reducing lipid, for example wherein the lipid-based carriers encapsulate the RNA, wherein the RNA comprises or consists of an RNA sequence identical or at least 70%, 80%, 90%, 91%, 92%, 93%, 94%, 95%, 96%, 97%, 98%, or 99% identical to any one of SEQ ID NOs: 673-695, 698-720, 723-745, 748-770, 773-795, 798-820, 823-845, 848-870, 873-895, 898-920, 923-945, 948-970, 973-995, 998-1020, 1023-1045, 1048-1070, 1073-1095, 1098-1120, 1123-1145, 1148-1170, 1173-1195, 1198-1220, 1223-1245, 1248-1270, 1271-1276 or a fragment or a variant thereof. In such embodiments, the RNA is for example an mRNA that comprises a cap1 structure and an RNA sequence wherein all uracils are substituted by pseudouridine (ψ) or N1-methylpseudouridine (m1ψ).

mRNA sequences in that context are for example SEQ ID NOs: 829, 679, 1271, 1274 or a fragment or a variant of any of these. Other mRNA sequences in that context are SEQ ID NOs: 834, 684, 1273, 1276 or a fragment or a variant of any of these. Other mRNA sequences in that context are SEQ ID NOs: 833, 683, 1272, 1275 or a fragment or a variant thereof.

In some embodiments, the wt/wt ratio of lipid to RNA in the lipid-based carrier is from about 10:1 to about 60:1, e.g. about 40:1. In some embodiments, the wt/wt ratio of lipid to RNA is from about 20:1 to about 30:1, e.g. about 25:1. In other embodiments, the wt/wt ratio of lipid to RNA is in the range of 20 to 60, for example from about 3 to about 15, about 5 to about 13, about 4 to about 8 or from about 7 to about 11.

The amount of lipid comprised in the lipid-based carriers may be selected taking the amount of the RNA cargo into account. In one embodiment, these amounts are selected such as to result in an N/P ratio of the lipid-based carriers encapsulating the RNA in the range of about 0.1 to about 20. The N/P ratio is defined as the mole ratio of the nitrogen atoms (“N”) of the basic nitrogen-containing groups of the lipid to the phosphate groups (“P”) of the RNA which is used as cargo. The N/P ratio may be calculated on the basis that, for example, 1 μg RNA typically contains about 3 nmol phosphate residues, provided that the RNA exhibits a statistical distribution of bases. The “N”-value of the lipid or lipidoid may be calculated on the basis of its molecular weight and the relative content of permanently cationic and—if present—cationisable groups.

In embodiments, the N/P ratio can be in the range of about 1 to about 50. In other embodiments, the range is from about 1 to about 20, and for example about 1 to about 15, from about 1 to about 10, or from about 5 to about 7. For “GN-LNPs”, a suitable N/P (lipid to RNA mol ratio) is about 14 or about 17. For “315-LNPs”, a suitable N/P (lipid to RNA mol ratio) is about 6. Another suitable N/P ratio is about 4.85 or 5 (lipid to RNA mol ratio).

In various embodiments, the pharmaceutical composition comprises lipid-based carriers (encapsulating RNA) that have a defined size (particle size, homogeneous size distribution).

The size of the lipid-based carriers of the pharmaceutical composition is typically described herein as Z-average size. The terms “average diameter”, “mean diameter”, “diameter” or “size” for particles (e.g. lipid-based carrier) are used synonymously with the value of the Z-average. The term “Z-average size” refers to the mean diameter of particles as measured by dynamic light scattering (DLS) with data analysis using the so-called cumulant algorithm, which provides as results the so-called Z-average with the dimension of a length, and the polydispersity index (PI), which is dimensionless (Koppel, D., J. Chem. Phys. 57, 1972, pp 4814-4820, ISO 13321).

The term “dynamic light scattering” or “DLS” refers to a method for analyzing particles in a liquid, wherein the liquid is typically illuminated with a monochromatic light source and wherein the light scattered by particles in the liquid is detected. DLS can thus be used to measure particle sizes in a liquid. Suitable DLS protocols are known in the art. DLS instruments are commercially available (such as the Zetasizer Nano Series, Malvern Instruments, Worcestershire, UK). DLS instruments employ either a detector at 90° (e.g. DynaPro® NanoStar® from Wyatt Technology or Zetasizer Nano S90® from Malvern Instruments) or a backscatter detection system at 173° (e.g., Zetasizer Nano S® from Malvern Instruments) and at 158° (DynaPro Plate Reader® from Malvern Instruments) close to the incident light of 180°. Typically, DLS measurements are performed at a temperature of about 25° C. DLS is also used in the context of the present disclosure to determine the polydispersity index (PDI) and/or the main peak diameter of the lipid-based carriers incorporating RNA.

In various embodiments, the lipid-based carriers of the pharmaceutical composition encapsulating RNA have a Z-average size ranging from about 50 nm to about 200 nm, from about 50 nm to about 190 nm, from about 50 nm to about 180 nm, from about 50 nm to about 170 nm, from about 50 nm to about 160 nm, 50 nm to about 150 nm, 50 nm to about 140 nm, 50 nm to about 130 nm, 50 nm to about 120 nm, 50 nm to about 110 nm, 50 nm to about 100 nm, 50 nm to about 90 nm, 50 nm to about 80 nm, 50 nm to about 70 nm, 50 nm to about 60 nm, 60 nm to about 200 nm, from about 60 nm to about 190 nm, from about 60 nm to about 180 nm, from about 60 nm to about 170 nm, from about 60 nm to about 160 nm, 60 nm to about 150 nm, 60 nm to about 140 nm, 60 nm to about 130 nm, 60 nm to about 120 nm, 60 nm to about 110 nm, 60 nm to about 100 nm, 60 nm to about 90 nm, 60 nm to about 80 nm, or 60 nm to about 70 nm, for example about 50 nm, 55 nm, 60 nm, 65 nm, 70 nm, 75 nm, 80 nm, 85 nm, 90 nm, 95 nm, 100 nm, 105 nm, 110 nm, 115 nm, 120 nm, 125 nm, 130 nm, 135 nm, 140 nm, 145 nm, 150 nm, 160 nm, 170 nm, 180 nm, 190 nm, or 200 nm.

In one embodiment, the lipid-based carriers of the pharmaceutical composition encapsulating RNA have a Z-average size ranging from about 50 nm to about 200 nm, for example in a range from about 50 nm to about 150 nm, for example from about 50 nm to about 120 nm.

Suitably, the pharmaceutical composition comprises less than about 10%, 9%, 8%, 7%, 6%, 5%, 4%, 3%, 2%, 1% lipid-based carriers that have a particle size exceeding about 500 nm.

Suitably, the pharmaceutical composition comprises less than about 10%, 9%, 8%, 7%, 6%, 5%, 4%, 3%, 2%, 1% LNPs that have a particle size smaller than about 20 nm.

Suitably, at least about 80%, 85%, 90%, 95% of lipid-based carriers of the composition have a spherical morphology.

In embodiments, the polydispersity index (PDI) of the lipid-based carriers is typically in the range of 0.1 to 0.5. In a particular embodiment, a PDI is below 0.2. Typically, the PDI is determined by dynamic light scattering.

In some embodiments, 80% of RNA comprised in the pharmaceutical composition is encapsulated in lipid based carriers, for example 85% of the RNA comprised in the pharmaceutical composition is encapsulated in lipid based carriers, for example 90% of the RNA comprised in the pharmaceutical composition is encapsulated in lipid based carriers, for example 95% of the RNA comprised in the pharmaceutical composition is encapsulated in lipid based carriers. The percentage of encapsulation may be determined by a RiboGreen assay as known in the art.

According to some embodiments the lipid-based carriers for example encapsulating or comprising RNA have been purified by at least one purification step, for example by at least one step of TFF and/or at least one step of clarification and/or at least one step of filtration.

### Antagonists of RNA Sensing Pattern Recognition Receptors:

In some embodiments, the pharmaceutical composition comprises at least one antagonist of at least one RNA sensing pattern recognition receptor. Such an antagonist may for example be co-formulated in lipid-based carriers as defined herein.

Suitable antagonist of at least one RNA sensing pattern recognition receptor are disclosed in published PCT patent application WO2021028439, the full disclosure herewith incorporated by reference. In particular, the disclosure relating to suitable antagonist of at least one RNA sensing pattern recognition receptors as defined in any one of the claims 1 to 94 of WO2021028439 are incorporated by reference.

In some embodiments in that context, the pharmaceutical composition comprises at least one antagonist of at least one RNA sensing pattern recognition receptor selected from a Toll-like receptor, for example TLR7 and/or TLR8.

In embodiments in that context, the at least one antagonist of at least one RNA sensing pattern recognition receptor is selected from a nucleotide, a nucleotide analogue, a nucleic acid, a peptide, a protein, a small molecule, a lipid, or a fragment, variant or derivative of any of these.

In some embodiments in that context, the at least one antagonist of at least one RNA sensing pattern recognition receptor is a single stranded oligonucleotide, for example a single stranded RNA Oligonucleotide.

In embodiments in that context, the at least one antagonist of at least one RNA sensing pattern recognition receptor is a single stranded oligonucleotide that comprises or consists of a nucleic acid sequence identical or at least 70%, 80%, 85%, 86%, 87%, 88%, 89%, 90%, 91%, 92%, 93%, 94%, 95%, 96%, 97%, 98%, or 99% identical to a nucleic acid sequence selected from the group consisting of SEQ ID NOs: 85-212 of WO2021028439, or fragments of any of these sequences.

In some embodiments in that context, the antagonist of at least one RNA sensing pattern recognition receptor is a single stranded oligonucleotide that comprises or consists of a nucleic acid sequence identical or at least 70%, 80%, 85%, 86%, 87%, 88%, 89%, 90%, 91%, 92%, 93%, 94%, 95%, 96%, 97%, 98%, or 99% identical to a nucleic acid sequence selected from the group consisting of SEQ ID NOs: 85-87, 149-212 of WO2021028439, or fragments of any of these sequences.

A suitable antagonist of at least one RNA sensing pattern recognition receptor in the context of the disclosure is 5′-GAG CGmG CCA-3′ (SEQ ID NO: 85 of WO2021028439), or a fragment thereof.

In some embodiments in that context, the molar ratio of the at least one antagonist of at least one RNA sensing pattern recognition receptor as defined herein to the at least one RNA encoding an antigenic peptide or protein as defined herein suitably ranges from about 1:1, to about 100:1, or ranges from about 20:1, to about 80:1.

In some embodiments in that context, the weight to weight ratio of the at least one antagonist of at least one RNA sensing pattern recognition receptor as defined herein to the at least one RNA encoding an antigenic peptide or protein as defined herein suitably ranges from about 1:1, to about 1:30, or ranges from about 1:2, to about 1:10.

In embodiments in that context, the at least one antagonist of at least one RNA sensing pattern recognition receptor as defined herein and the at least one RNA encoding an antigenic peptide or protein as defined herein are separately formulated, for example separately formulated in lipid-based carriers as defined herein.

In some embodiments in that context, the at least one antagonist of at least one RNA sensing pattern recognition receptor as defined herein and the at least one RNA encoding an antigenic peptide or protein as defined herein are co-formulated, for example co-formulated in lipid-based carriers as defined herein.

In embodiments of composition comprising at least one antagonist of at least one RNA sensing pattern recognition receptor, the at least one RNA encoding an antigenic peptide or protein as defined herein does for example not comprise chemically modified nucleotides (e.g. pseudouridine (ψ) or N1-methylpseudouridine (m1ψ)) as defined herein.

### Presentation:

In some embodiments, the pharmaceutical composition is lyophilized, spray-dried or spray-freeze dried.

Accordingly, the pharmaceutical composition is lyophilized (e.g. according to WO2016165831 or WO2011069586) to yield a temperature stable composition. The pharmaceutical composition may also be dried using spray-drying or spray-freeze drying (e.g. according to WO2016184575 or WO2016184576) to yield a temperature stable composition (powder) as defined herein.

Lyoprotectants for lyophilization and or spray drying may be selected from trehalose, sucrose, mannose, dextran and inulin. A suitable lyoprotectant is sucrose, optionally comprising a further lyoprotectant. A further suitable lyoprotectant is trehalose, optionally comprising a further lyoprotectant. Accordingly, the pharmaceutical composition may comprise at least one lyoprotectant.

In some embodiments, the pharmaceutical composition is a liquid composition or dried composition, such as a lyophilized/spry-dried composition which can be reconstituted in a liquid carrier.

In some embodiments, the pharmaceutical composition (or the liquid carrier) comprises a sugar in a concentration of about 50 mM to about 300 mM, for example sucrose in a concentration of about 150 mM.

In some embodiments, the pharmaceutical composition (or the liquid carrier) comprises a salt in a concentration of about 10 mM to about 200 mM, for example NaCl in a concentration of about 75 mM.

In some embodiments, the pharmaceutical composition (or the liquid carrier) comprises a buffering agent in a concentration 1 mM to about 100 mM, for example Na₂HPO₄, Na₃PO₄ or Tris (Trometamol). In other embodiments, the pharmaceutical composition (or the liquid carrier) comprises about 2.4 mM Tris (Trometamol), about 1.4 mM glacial acetic acid, about 3.9 mM acetic acid and about 254 mM sugar.

In some embodiments, the pharmaceutical composition (or the liquid carrier) has a pH in a range of about pH 7.0 to about pH 8.0, for example of about pH 7.4.

3: Vaccine Comprising a Coding RNA Encoding an Antigenic Polypeptide which is or is Derived from E. coli FimH

In a third aspect, it is provided a vaccine against E. coli.

Notably, embodiments relating to the composition of the second aspect may likewise be read on and be understood as suitable embodiments of the vaccine of the third aspect. Also, embodiments relating to the vaccine of the third aspect may likewise be read on and be understood as suitable embodiments of the composition of the second aspect. Furthermore, features and embodiments described in the context of the first aspect (the coding RNA of the disclosure) have to be read on and have to be understood as suitable embodiments of the vaccine of the third aspect.

In some embodiments, the vaccine comprises a coding RNA of the first aspect, or at least one composition of the second aspect.

The term “vaccine” will be recognized and understood by the person of ordinary skill in the art, and is for example intended to be a prophylactic or therapeutic material providing at least one epitope or antigen, for example an immunogen. In the context of the disclosure the antigen or antigenic function is suitably provided by the RNA of the first aspect (said coding RNA comprising a coding sequence encoding an antigenic polypeptide which is selected or derived from E. coli FimH) or the composition of the second aspect (comprising a coding RNA of the first aspect).

In some embodiments, the vaccine elicits an adaptive immune response, for example a protective adaptive immune response against E. coli. Particularly, the E. coli is selected from the group consisting of: E. coli J96, E. coli 536, E. coli CFT073, E. coli UMN026, E. coli CLONE D i14, E. coli CLONE D i2, E. coli IA139, E. coli NA114, E. coli IHE3034, E. coli 789, E. coli F11 and E. coli UTI89.

In some embodiments, the vaccine, for example the administration of the vaccine to a subject, elicits a humoral immune response against E. coli. In one embodiment, said humoral immune response is against E. coli FimH. In one embodiment, the vaccine, for example the administration of the vaccine to a subject, elicits a humoral immune response against E. coli, for example against E. coli FimH, in the urine of a subject upon administration of the vaccine.

In one embodiment, the vaccine, for example the administration of the vaccine to a subject, elicits neutralizing antibody titers against E. coli. In one embodiment, said antibodies are IgG antibodies. In one embodiment, said antibodies are against E. coli FimH. In one embodiment, the vaccine, for example the administration of the vaccine to a subject, elicits neutralizing antibody titers against E. coli, for example against E. coli FimH, in the urine of a subject upon administration of the vaccine.

In one embodiment, the vaccine of the disclosure elicits antibodies which are capable of inhibiting bacterial adhesion to uroepithelial cells. Suitable methods for measuring inhibition of bacterial adhesion are described herein and in the Examples.

Methods for measuring bacterial adhesion are known in the art. Suitably, the method described in Thomas W E, et al. Cell. 2002 Jun. 28; 109(7):913-23, which is incorporated by reference herein; or Hartmann M, et al. FEBS Lett. 2012 May 21; 586(10):1459-65, which is incorporated by reference herein; or Falk P, et sl. Methods Cell Biol. 1994; 45:165-92, which is incorporated by reference herein; or Garcia Mendez K B, et al. Int J Exp Pathol. 2016 April; 97(2):194-201, which is incorporated by reference herein, may be used. In one embodiment, bacterial adhesion is (in brief) measured with the BAI assay as follows and as described in the Examples: UPEC strains engineered to express the mCherry fluorescent marker, are incubated for 30 minutes with monolayers of SV-HUC-1 (ATTCC) in 96 well plates in the presence of specific sera against FimH derivatives or positive/negative controls. After adhesion, cells are washed extensively to remove unbound bacteria and fixed with formaldehyde. Finally, the specific fluorescent signal associated with the adhered bacteria is recorded by the use of an automated high content screening microscope (Opera Phenix) and quantified with the Harmony software.

In some embodiments, the immune response is effective to prevent or treat one or more symptoms associated with UTI in the subject in need thereof. In certain embodiments, the immune response is effective to prevent or reduce a symptom of UTI, for example in at least 30%, for example at least 40%, such as at least 50%, of the subjects administered with the vaccine. Symptoms of UTI can vary depending on the nature of the infection and can include, but are not limited to: dysuria, increased urinary frequency or urgency, pyuria, hematuria, back pain, pelvic pain, pain while urinating, fever, chills, and/or nausea.

In certain embodiments, the immune response is effective to prevent or reduce organ failure resulting from a UTI. In certain embodiments, the immune response is effective to reduce the likelihood of hospitalization of a subject suffering from a UTI. In some embodiments, the immune response effective to reduce the duration of hospitalization of a subject suffering from a UTI.

The vaccine may be used according to the disclosure for human medical purposes and also for veterinary medical purposes (mammals, vertebrates, or avian species).

Suitable administration routes for the vaccine comprise intranasal, oral, sublingual, intravenous, intramuscular, intradermal, transdermal, or subcutaneous. Accordingly, in some embodiments, the vaccine is suitable for intranasal, oral, sublingual, intravenous, intramuscular, intradermal, transdermal, or subcutaneous administration.

In one embodiment, the vaccine is suitable for intramuscular administration.

In one embodiment, the vaccine comprises lipid-based carriers comprising
(i) at least one cationic lipid selected from ALC-0315;
(ii) at least one neutral lipid selected from DSPC;
(iii) at least one steroid or steroid analogue selected from cholesterol; and
(iv) at least one aggregation reducing lipid selected from ALC-0159; and wherein the lipid-based carriers encapsulate the RNA of the disclosure, for example wherein i) to
(iv) are in a molar ratio of about 47.4% cationic lipid, about 10% neutral lipid, about 40.9% steroid or steroid analogue, and about 1.7% aggregation reducing lipid, wherein the RNA comprises or consists of an RNA sequence identical or at least 70%, 80%, 90%, 91%, 92%, 93%, 94%, 95%, 96%, 97%, 98%, or 99% identical to any one of SEQ ID NOs: 673-695, 698-720, 723-745, 748-770, 773-795, 798-820, 823-845, 848-870, 873-895, 898-920, 923-945, 948-970, 973-995, 998-1020, 1023-1045, 1048-1070, 1073-1095, 1098-1120, 1123-1145, 1148-1170, 1173-1195, 1198-1220, 1223-1245, 1248-1270, or a fragment or a variant thereof. In such embodiments, the RNA is for example an mRNA that comprises a cap1 structure and the RNA sequence is not chemically modified (e.g. consisting of non-modified ribonucleotides). mRNA sequences in that context are for example SEQ ID NOs: 829, 679, or a fragment or a variant of any of these. Other mRNA sequences in that context are SEQ ID NOs: 834 684, or a fragment or a variant of any of these. Other mRNA sequences in that context are SEQ ID NOs: 833, 683, or a fragment or a variant of any of these.

In one embodiment, the vaccine comprises lipid-based carriers comprising
(i) at least one cationic lipid selected from ALC-0315;
(ii) at least one neutral lipid selected from DSPC;
(iii) at least one steroid or steroid analogue selected from cholesterol; and
(iv) at least one aggregation reducing lipid selected from ALC-0159; and
wherein the lipid-based carriers encapsulate the RNA of the disclosure, for example wherein i) to (iv) are in a molar ratio of about 47.4% cationic lipid, about 10% neutral lipid, about 40.9% steroid or steroid analogue, and about 1.7% aggregation reducing lipid, wherein the RNA comprises or consists an RNA sequence identical or at least 70%, 80%, 90%, 91%, 92%, 93%, 94%, 95%, 96%, 97%, 98%, or 99% identical to any one of SEQ ID NOs: 673-695, 698-720, 723-745, 748-770, 773-795, 798-820, 823-845, 848-870, 873-895, 898-920, 923-945, 948-970, 973-995, 998-1020, 1023-1045, 1048-1070, 1073-1095, 1098-1120, 1123-1145, 1148-1170, 1173-1195, 1198-1220, 1223-1245, 1248-1270, 1274-1276 or a fragment or a variant thereof. In such embodiments, the RNA is for example an mRNA that comprises a cap1 structure and the uracils of the RNA sequence are substituted by pseudouridine (ψ). mRNA sequences in that context are for example SEQ ID NOs: 829, 679, 1274 or a fragment or a variant of any of these. Other mRNA sequences in that context are SEQ ID NOs: 834 684, 1276 or a fragment or a variant thereof. Other mRNA sequences in that context are SEQ ID NOs: 833, 683, 1275 or a fragment or a variant of any of these.

In one embodiment, the vaccine comprises lipid-based carriers comprising
(i) at least one cationic lipid selected from ALC-0315;
(ii) at least one neutral lipid selected from DSPC;
(iii) at least one steroid or steroid analogue selected from cholesterol; and
(iv) at least one aggregation reducing lipid selected from ALC-0159; and
wherein the lipid-based carriers encapsulate the RNA of the disclosure, for example wherein i) to (iv) are in a molar ratio of about 47.4% cationic lipid, about 10% neutral lipid, about 40.9% steroid or steroid analogue, and about 1.7% aggregation reducing lipid, wherein the RNA comprises or consists an RNA sequence identical or at least 70%, 80%, 90%, 91%, 92%, 93%, 94%, 95%, 96%, 97%, 98%, or 99% identical to any one of SEQ ID NOs: 673-695, 698-720, 723-745, 748-770, 773-795, 798-820, 823-845, 848-870, 873-895, 898-920, 923-945, 948-970, 973-995, 998-1020, 1023-1045, 1048-1070, 1073-1095, 1098-1120, 1123-1145, 1148-1170, 1173-1195, 1198-1220, 1223-1245, 1248-1270, 1271-1273 or a fragment or a variant thereof. In such embodiments, the RNA is for example an mRNA that comprises a cap1 structure and the uracils of the RNA sequence are substituted by N1-methylpseudouridine (m1ψ). mRNA sequences in that context are for example SEQ ID NOs: 829, 679, 1271 or a fragment or a variant of any of these. Other mRNA sequences in that context are SEQ ID NOs: 834 684, 1273 or a fragment or a variant of any of these. Other mRNA sequences in that context are SEQ ID NOs: 833, 683, 1272 or a fragment or a variant of any of these.

### 4: A Kit or Kit of Parts

In a fourth aspect, it is provided a kit or kit of parts suitable for treating or preventing an infection caused by Escherichia coli. Notably, embodiments relating to the RNA of the first aspect may likewise be read on and be understood as suitable embodiments of the kit or kit of parts of the fourth aspect. Also, embodiments relating to the pharmaceutical composition of the second aspect or the vaccine of the third aspect may likewise be read on and be understood as suitable embodiments of the kit or kit of parts of the fourth aspect,

In some embodiments, the kit or kit of parts comprises at least one coding RNA of the first aspect, at least one composition of the second aspect, and/or at least one vaccine of the third aspect.

In addition, the kit or kit of parts may comprise a liquid vehicle for solubilising, and/or technical instructions providing information on administration and dosage of the components.

The kit may further comprise additional components as described in the context of the composition of the second aspect, and/or the vaccine of the third aspect.

The technical instructions of said kit may contain information about administration and dosage and patient groups. Such kits, for example kits of parts, may be applied e.g. for any of the applications or uses mentioned herein, for example for the use of the RNA of the first aspect, the composition of the second aspect, the vaccine of the third aspect, for the treatment or prophylaxis of an infection or diseases caused by an Escherichia coli, or disorders related thereto.

Suitably, the coding RNA, the composition, or the vaccine is provided in a separate part of the kit.

In some embodiments, the coding RNA, the composition, or the vaccine is lyophilised or spray(freeze)dried.

In embodiments where the pharmaceutical composition is provided as a lyophilized or spray-freeze dried or spray dried composition, the kit or kit of parts may suitably comprise a buffer for re-constitution of lyophilized or spray-freeze dried or spray dried composition.

Accordingly, the kit or kit of parts may additionally comprise a buffer for re-constitution and/or dilution of the RNA, the composition, or the vaccine.

In some embodiments, the buffer for re-constitution and/or dilution is a sterile buffer. In some embodiments, the buffer comprises a salt, for example NaCl, optionally in a concentration of about 0.9%. Such a buffer may optionally comprise a preservative.

In some embodiments, the kit or kit of parts as defined herein comprises at least one syringe.

In some embodiments, the kit or kit of parts comprises the following components:
a) at least one container or vial comprising a composition or a vaccine as defined herein.
b) optionally, at least one dilution container or vial comprising a sterile dilution buffer, suitably a buffer comprising NaCl, optionally comprising a preservative;
c) optionally, at least one means for transferring the composition or vaccine from the container to the dilution container; and
d) at least one syringe for administering the composition or vaccine to a subject, for example a syringe configured for intramuscular administration to a human subject.

### 5: Medical Uses

In a further aspect, it is provided a medical use of the coding RNA as defined herein, the composition as defined herein, the vaccine as defined herein, or the kit or kit of parts as defined herein.

Notably, embodiments relating to the previous aspects may likewise be read on and be understood as suitable embodiments of medical uses of the invention (and vice versa).

Accordingly, it is provided the coding RNA of the disclosure, and/or the composition of the disclosure, and/or the vaccine of the disclosure, and/or the kit or kit of parts of the disclosure for use as a medicament

In a further aspect, second medical uses of the coding RNA as defined herein, the composition as defined herein, the vaccine as defined herein, or the kit or kit of parts as defined herein are provided.

Accordingly, it is provided the coding RNA of the disclosure, and/or the composition of the disclosure, and/or the vaccine of the disclosure, and/or the kit or kit of parts of the disclosure for use in treating or preventing a disease caused by E. coli. Particularly, the E. coli is selected from the group consisting of: E. coli J96, E. coli 536, E. coli CFT073, E. coli UMN026, E. coli CLONE D i14, E. coli CLONE D i2, E. coli IA139, E. coli NA114, E. coli IHE3034, E. coli 789, E. coli F11 and E. coli UTI89.

In some embodiments, it is provided the coding RNA of the disclosure, and/or the composition of the disclosure, and/or the vaccine of the disclosure, and/or the kit or kit of parts of the disclosure for use in treating or preventing one or more symptoms associated with UTI in the subject in need thereof. In certain embodiments, the use is for treating or preventing a symptom of UTI, for example in at least 30%, for example at least 40%, such as at least 50%, of the subjects administered with the vaccine. Symptoms of UTI can vary depending on the nature of the infection and can include, but are not limited to: dysuria, increased urinary frequency or urgency, pyuria, hematuria, back pain, pelvic pain, pain while urinating, fever, chills, and/or nausea.

In certain embodiments, the use is for preventing or reducing organ failure resulting from a UTI. In certain embodiments, the use is for reducing the likelihood of hospitalization of a subject suffering from a UTI. In some embodiments, the use is for reducing the duration of hospitalization of a subject suffering from a UTI.

In the context of a medical use, the coding RNA of the disclosure, and/or the composition of the disclosure, and/or the vaccine of the disclosure, and/or the kit or kit of parts of the disclosure may for example be administered locally.

In that context, administration may be by an intranasal, oral, sublingual, intravenous, intramuscular, intradermal, transdermal, or subcutaneous, for example intramuscular.

In one embodiment, administration may be by a conventional needle injection, e.g. an intramuscular injection.

In some embodiments, the use may be for human medical purposes and also for veterinary medical purposes. In one embodiment, the use may for human medical purposes.

In some embodiments, the use is for a naive subject, i.e., a subject that does not have an E. coli infection or has not previously had a UTI. In one embodiment, the use is for a subject that is at risk of acquiring or developing a UTI, e.g., an immunocompromised or immunodeficient individual, before symptoms manifest or symptoms become severe. In certain embodiments, the use is for a subject who has been or was previously diagnosed with a UTI.

As used herein, the term “at-risk subject” refers to a human that is more prone to a condition than the average human adult population. Examples of an “at-risk subject” include persons that have one or more risk factors for UTI which can include, but are not limited to, elderly people, immunocompromised people, people with diabetes, people with known history of rUTI, people with obstructions in the urinary tract such as kidney stones, sexually active women, women after menopause, people using a catheter, people that are incontinent, people recently having undergone a urinary system procedure such as surgery on the urinary tract, etc.

In certain embodiments, the use is for a subject who has been or was previously diagnosed with an UPEC infection. In some embodiments, the use is for a subject suffering from reoccurring UTIs. In some embodiments, the use is for a subject suffering from reoccurring UTIs, but is healthy at the moment of treatment. In some embodiments, the use is for a subject having or at risk of acquiring E. coli bacteremia or sepsis. In some embodiments, a subject to be administered or applied a composition or method of the disclosure has a condition that requires them to use a catheter, such as a urinary catheter (which leads to risk of CAUTI, i.e. catheter associated UTI). In some embodiments, the use is for a subject that undergoes a pre-scheduled surgery.

In certain embodiments, the use is a human adult more than 50 years old. In certain embodiments, the use is for a human adult more than 55, more than 60 or more than 65 years old. In certain embodiments, the use is for a woman between age of about 16 to 50 years old, e.g. between age of about 16 and 35 years old. In certain embodiments, the use is for a subject that has diabetes.

### 6: Method of Treatment

In a further aspect, it is provided a method of treating or preventing a disease caused by E. coli. The method comprises administering to a subject in need thereof an effective amount of the coding RNA according to the first aspect, the pharmaceutical composition according to the second aspect, the vaccine according to the third aspect, or the kit or kit of parts according to the fourth aspect. It is also provided a method for inducing an immune response in a subject in need thereof. Suitably, the immune response is effective to prevent or treat one or more symptoms associated with UTI in the subject in need thereof.

It is also provided the use of the coding RNA according to the first aspect, the pharmaceutical composition according to the second aspect, the vaccine according to the third aspect or the kit or kit of parts according to the fourth aspect for raising an immune response in a mammal, for example, for treating and/or preventing a disease. It is also provided the use of the coding RNA according to the first aspect, the pharmaceutical composition according to the second aspect, the vaccine according to the third aspect or the kit or kit of parts according to the fourth aspect for the manufacture of a medicament for raising an immune response in a mammal, for example, for treating and/or preventing a disease, for example an E. coli infection.

Notably, embodiments relating to the previous aspects may likewise be read on and be understood as suitable embodiments of medical uses of the disclosure.

Furthermore, specific features and embodiments relating to method of treatments as provided herein may also apply for medical uses of the disclosure and vice versa.

Preventing (inhibiting) or treating a disease, in particular an infection caused by E. coli relates to inhibiting the full development of a disease or condition, for example, in a subject who is at risk for a disease such as an infection. “Treatment” refers to a therapeutic intervention that ameliorates a sign or symptom of a disease or pathological condition after it has begun to develop. The term “ameliorating”, with reference to a disease or pathological condition, refers to any observable beneficial effect of the treatment. Inhibiting a disease can include preventing or reducing the risk of the disease, such as preventing or reducing the risk of an infection with E. coli. The beneficial effect can be evidenced, for example, by a delayed onset of clinical symptoms of the disease in a susceptible subject, a reduction in severity of some or all clinical symptoms of the disease, a slower progression of the disease, a reduction in the viral load, an improvement in the overall health or well-being of the subject, or by other parameters that are specific to the particular disease. A “prophylactic” treatment is a treatment administered to a subject who does not exhibit signs of a disease or exhibits only early signs for the purpose of decreasing the risk of developing pathology.

In some embodiments, it is provided a method of treating or preventing a disease, disorder or condition, wherein the method comprises applying or administering to a subject in need thereof the coding RNA of the disclosure, and/or the composition of the disclosure, and/or the vaccine of the disclosure, and/or the kit or kit of parts of the disclosure.

In some embodiments, the disease, disorder or condition is an infectious disease caused by E. coli or a disorder related to such an infectious disease.

In a specific embodiment, the methods of inducing an immune response in a subject of the disclosure result in vaccination of the subject to induce a protective immunity against infection by the E. coli strains expressing FimH.

In one embodiment, the method induces a humoral immune response against E. coli.

In one embodiment, said humoral immune response is against E. coli FimH. In one embodiment, the method elicits a humoral immune response against E. coli, for example against E. coli FimH, in the urine of a subject.

In one embodiment, the method elicits neutralizing antibody titers against E. coli. In one embodiment, said antibodies are IgG antibodies. In one embodiment, said antibodies are against E. coli FimH. In one embodiment, the method elicits neutralizing antibody titers against E. coli, for example against E. coli FimH, in the urine of a subject.

In one embodiment, the methods of the disclosure elicit antibodies which are capable of inhibiting bacterial adhesion to uroepithelial cells. Suitable methods for measuring inhibition of bacterial adhesion are described herein and in the Examples.

In certain embodiments, the immune response induced in a subject following administration of the coding RNA, the pharmaceutical composition or the vaccine according of the disclosure is effective to eliminate a UTI.

In certain embodiments, the immune response induced in a subject following administration of the immune response induced in a subject following administration of the coding RNA, the pharmaceutical composition or the vaccine according of the disclosure is effective to prevent or reduce a symptom of UTI, for example in at least 30%, for example at least 40%, such as at least 50%, of the subjects administered with the composition. Symptoms of UTI can vary depending on the nature of the infection and can include, but are not limited to: dysuria, increased urinary frequency or urgency, pyuria, hematuria, back pain, pelvic pain, pain while urinating, fever, chills, and/or nausea.

In certain embodiments, the immune response induced in a subject following administration of the immune response induced in a subject following administration of the coding RNA, the pharmaceutical composition or the vaccine according of the disclosure is effective to prevent or reduce organ failure resulting from a UTI. In certain embodiments, the immune response induced in a subject following administration of the immune response induced in a subject following administration of the coding RNA, the pharmaceutical composition or the vaccine according of the disclosure is effective to reduce the likelihood of hospitalization of a subject suffering from a UTI. In some embodiments, the immune response induced in a subject following administration of a composition of the disclosure is effective to reduce the duration of hospitalization of a subject suffering from a UTI.

In some embodiments, applying or administering is performed via nasal administration, oral administration, sublingual administration, intramuscular injection, intravenous injection, transdermal injection, or intradermal injection. In one embodiment, applying or administering is performed via intramuscular injection.

As used herein in the context of the disclosure, the term “effective amount” refers to the amount that is sufficient to induce a desired immune effect or immune response in the subject. In certain embodiments, an “effective amount” refers to the amount which is sufficient to produce immunity in a subject to achieve one or more of the following effects in the subject: (i) prevent the development or onset of a UTI or symptom associated therewith; (ii) prevent or reduce the recurrence of a UTI or symptom associated therewith; (iii) prevent, reduce or ameliorate the severity of a UTI or symptom associated therewith; (iv) reduce the duration of infection UTI or symptom associated therewith; (v) prevent the clinical progression of a UTI or symptom associated therewith; (vi) cause regression of a UTI or symptom associated therewith; (vii) prevent or reduce organ failure resulting from UTI; (viii) reduce the chance or frequency of hospitalization of a subject having a UTI; (ix) reduce hospitalization length of a subject having a UTI; (x) eliminate a UTI; and/or (xi) enhance or improve the prophylactic or therapeutic effect(s) of another therapy.

Selection of a particular effective dose can be determined (e.g., via clinical trials) by those skilled in the art based upon the consideration of several factors, including the disease to be treated or prevented, the symptoms involved, the medical history of the subject, the physical condition of the subject, such as the subject's age, weight and/or immune status, the composition administered, and other factors known by the skilled artisan. The precise dose to be employed in the formulation will also depend on the route of administration, and the severity of disease, and should be decided according to the judgment of the practitioner and each patient's circumstances. Effective doses can be extrapolated from dose-response curves derived from in vitro or animal model test systems.

In one embodiment, the subject in need is a mammalian subject, for example a human subject.

In certain embodiments, a method of the disclosure is administered or applied to a naive subject, i.e., a subject that does not have an E. coli infection or has not previously had a UTI. In one embodiment, a composition or method of the disclosure is administered or applied to a subject that is at risk of acquiring or developing a UTI, e.g., an immunocompromised or immunodeficient individual, before symptoms manifest or symptoms become severe. In certain embodiments, method of the disclosure is administered or applied to a subject who has been or was previously diagnosed with a UTI.

In certain embodiments, a method of the disclosure is administered or applied to a subject who has been or was previously diagnosed with a UPEC infection. In some embodiments, a composition or method of the disclosure is administered or applied to a subject suffering from reoccurring UTIs. In some embodiments, a method of the invention is administered or applied to a subject suffering from reoccurring UTIs, but is healthy at the moment of treatment. In some embodiments, a method of the disclosure is administered or applied to a subject having or at risk of acquiring E. coli bacteremia or sepsis. In some embodiments, a subject to be applied a method of the disclosure has a condition that requires them to use a catheter, such as a urinary catheter (which leads to risk of CAUTI, i.e. catheter associated UTI). In some embodiments, a method of the disclosure is applied to a subject that undergoes a pre-scheduled surgery.

In certain embodiments, a subject to be applied a method of the disclosure is a human subject, for example, a human subject at risk of having disease UTI. In certain embodiments, a subject to be applied a composition or method of the disclosure is a human adult more than 50 years old. In certain embodiments, a subject to be applied a method of the disclosure is a human adult more than 55, more than 60 or more than 65 years old.

In certain embodiments, a subject to be applied a method of the disclosure is a woman between age of about 16 to 50 years old, e.g. between age of about 16 and 35 years old. In certain embodiments, a subject to be applied a method of the disclosure has diabetes.

### BRIEF DESCRIPTION OF TABLES

Table 1: Sequences (amino acid sequences and coding sequences)
Table 2: RNA constructs
Table 3: RNA constructs encoding the antigen designs used in the examples
Table 4: Lipid-based carrier composition of the examples
Table 5: RNA constructs used for Western blot analysis (Example 2.1)
Table 6: Vaccination regimen (Example 2.2)
Table 7: BAI titers of serum antibody responses against E. coli FimH antigen designs (Example 2.3)
Table 8: Vaccination regimen (Example 3.1)
Table 9: BAI titers of serum antibody responses against E. coli FimH antigen designs (Example 3.2)
Table 10: mRNA constructs used for Western blot analysis (Example 4.1)
Table 11: Vaccination regimen (Example 4.2)
Table 12: BAI titers of serum antibody responses against E. coli FimH antigen designs (Example 4.3)
Table 13: additional sequences shorter than 10 specifically defined nucleotides or 4 specifically defined amino acid.

### NUMBERED EMBODIMENTS

In the following, embodiments of the present invention are provided as a numbered embodiments list (embodiment 1 to embodiment 110).

Embodiment 1. A coding RNA comprising at least one untranslated region (UTR) and at least one coding sequence encoding an antigenic polypeptide which is selected or derived from Escherichia coli type 1 fimbriae D-mannose specific adhesin (FimH).

Embodiment 2. The coding RNA according to embodiment 1, wherein the E. coli FimH comprises an amino acid sequence which is identical or at least 70%, 80%, 85%, 86%, 87%, 88%, 89%, 90%, 91%, 92%, 93%, 94%, 95%, 96%, 97%, 98%, or 99% identical to any one of SEQ ID NOs: 177-186, 247-256 or is an immunogenic fragment or immunogenic variant thereof.

Embodiment 3. The coding RNA according to embodiments 1 or 2, wherein the coding sequence additionally encodes one or more further peptide or protein elements selected from: a donor strand peptide, a signal peptide, an antigen clustering domain, or a transmembrane domain.

Embodiment 4. The coding RNA according to embodiment 3, wherein the one or more further peptide or protein element(s) is a donor strand peptide, optionally wherein the coding sequence encodes the following elements in N-terminal to C-terminal direction: the antigenic polypeptide which is selected or is derived from E. coli FimH; and the donor strand peptide.

Embodiment 5. The coding RNA according to embodiment 4, wherein the donor strand peptide comprises or consists of the amino acid sequence of SEQ ID NO: 338 or SEQ ID NO: 339 or a variant thereof, optionally wherein the variant of SEQ ID NO: 338 or SEQ ID NO: 339 has from 1 to 5, such as 1, 2, 3 or 4 single amino acid mutations compared to SEQ ID NO: 338 or SEQ ID NO: 339.

Embodiment 6. The coding RNA according to embodiment 4, wherein the donor strand peptide comprises or consists of the amino acid sequence of SEQ ID NO: 338.

Embodiment 7. The coding RNA according to any one of embodiments 1 to 6, wherein the coding sequence additionally encodes a peptide linker.

Embodiment 8. The coding RNA according to embodiment 1 to 7, wherein the coding sequence encodes the following elements in N-terminal to C-terminal direction: the antigenic polypeptide which is selected or derived from E. coli FimH; the peptide linker element; and the donor strand peptide.

Embodiment 9. The coding RNA according to embodiments 7 or 8, wherein the peptide linker comprises or consists of any one of SEQ ID NOs: 352-358.

Embodiment 10. The coding RNA according to embodiments 7 to 9, wherein the peptide linker comprises or consists of SEQ ID NO: 352.

Embodiment 11. The coding RNA of any one of the preceding embodiments, wherein the antigenic polypeptide is in a low mannose binding affinity conformation.

Embodiment 12. The coding RNA according to any one of embodiments 1 to 11, wherein the coding sequence additionally encodes an antigen clustering domain.

Embodiment 13. The coding RNA according to embodiment 12, wherein the antigen clustering domain is selected or derived from ferritin or lumazine synthase.

Embodiment 14. The coding RNA according to any one of embodiments 12 or 13, wherein the amino acid sequence of the antigen clustering domain is identical or at least 70%, 80%, 85%, 86%, 87%, 88%, 89%, 90%, 91%, 92%, 93%, 94%, 95%, 96%, 97%, 98%, or 99% identical to any one of amino acid sequences SEQ ID NOs: 457-459, 443, 444, or fragment or variant thereof.

Embodiment 15. The coding RNA according to any one of embodiments 1 to 11, wherein the coding sequence additionally encodes a transmembrane domain.

Embodiment 16. The coding RNA according to claim 15, wherein the transmembrane domain is heterologous and is optionally selected or derived is or is derived from an influenza HA transmembrane domain, for example from SEQ ID NO: 478.

Embodiment 17. The coding RNA according to any one of embodiments 1 to 16, wherein the coding sequence additionally encodes a signal peptide.

Embodiment 18. The coding RNA according to embodiment 17, wherein the signal peptide is selected or derived from FimH, FimC, immunoglobulin Kappa IgK (IgK), immunoglobulin IgE (IgE), tissue plasminogen activator (TPA or HsPLAT), human serum albumin (HSA or HsALB), or MHC class I lymphocyte antigen (HLA-A2), optionally wherein the amino acid sequences of the signal peptides is identical or at least 70%, 80%, 85%, 86%, 87%, 88%, 89%, 90%, 91%, 92%, 93%, 94%, 95%, 96%, 97%, 98%, or 99% identical to any one of amino acid sequences SEQ ID NOs: 394-400, or fragment or variant thereof.

Embodiment 19. The coding RNA according to embodiments 17 or 18, wherein the signal peptide is selected or derived from IgE or IgK, optionally wherein the amino acid sequences of said signal peptides is identical or at least 70%, 80%, 85%, 86%, 87%, 88%, 89%, 90%, 91%, 92%, 93%, 94%, 95%, 96%, 97%, 98%, or 99% identical to any one of amino acid sequences SEQ ID NOs: 394, 395, or fragment or variant thereof.

Embodiment 20 (a). The coding RNA according to any one of embodiments 1 to 19, wherein the coding sequence encodes the following elements for example in N-terminal to C-terminal direction:
a) a signal peptide, the antigenic polypeptide;
b) a signal peptide, the antigenic polypeptide, a peptide linker, a donor strand peptide;
c) an antigen clustering domain, a peptide linker, the antigenic polypeptide, a peptide linker, a donor strand peptide;
d) a signal peptide, an antigen clustering domain, a peptide linker, the antigenic polypeptide, a peptide linker, a donor strand peptide;
e) a signal peptide, the antigenic polypeptide, a peptide linker, a donor strand peptide, a peptide linker, an antigen clustering domain; or
f) a signal peptide, the antigenic polypeptide, a peptide linker, a donor strand peptide, a peptide linker, a transmembrane domain.

Embodiment 20 (b). The coding RNA according to any one of embodiments 1 to 19 and 20 (a), wherein the coding sequence encodes the following elements for example in N-terminal to C-terminal direction: a signal peptide, the antigenic polypeptide, a peptide linker, and a donor strand peptide; optionally wherein the signal peptide is selected from SEQ ID NOs: 394-400, optionally wherein the signal peptide is SEQ ID NO: 395; the antigenic polypeptide is selected from SEQ ID NOs: 247-256, optionally wherein the antigenic polypeptide is SEQ ID NO: 247; the peptide linker is selected from SEQ ID NOs: 352-354, optionally wherein the peptide linker is SEQ ID NO: 352; the donor strand peptide is selected from SEQ ID NOs: 338, 339, optionally wherein the donor strand peptide is SEQ ID NO: 338.

Embodiment 21. The coding RNA according to any one of embodiments 1 to 19, wherein the coding sequence encodes the following elements for example in N-terminal to C-terminal direction: a signal peptide, the antigenic polypeptide as defined herein, a (first) peptide linker, a donor strand peptide, a (second) peptide linker; and an antigen clustering domain; optionally wherein the signal peptide is selected from SEQ ID NOs: 394-400, optionally wherein the signal peptide is SEQ ID NO: 394; the antigenic polypeptide is selected from SEQ ID NOs: 247-256, optionally wherein the antigenic polypeptide is SEQ ID NO: 247; the (first) peptide linker is selected from SEQ ID NOs: 352-354, optionally wherein the (first) peptide linker is SEQ ID NO: 352; the donor strand peptide is selected from SEQ ID NOs: 338, 339, optionally wherein the donor strand peptide is SEQ ID NO: 338; the (second) peptide linker is selected from SEQ ID NOs: 355-358, optionally wherein the (second) peptide linker is SEQ ID NO: 355; the antigen clustering domain is selected from SEQ ID NOs: 443, 444, 457-459, optionally wherein the peptide linker is SEQ ID NOs: 444 or 459.

Embodiment 22. The coding RNA according to any one of the preceding embodiments, wherein the coding sequence encodes an amino acid sequence which is identical or at least 70%, 80%, 85%, 86%, 87%, 88%, 89%, 90%, 91%, 92%, 93%, 94%, 95%, 96%, 97%, 98%, or 99% identical to any one of SEQ ID NOs: 177-186, 247-256, 498-520, 1277, or an immunogenic fragment or immunogenic variant thereof.

Embodiment 23 (a). The coding RNA according to any one of the preceding embodiments, wherein the coding sequence encodes an amino acid sequence which is identical or at least 90%, 91%, 92%, 93%, 94%, 95%, 96%, 97%, 98%, or 99% identical to any one of SEQ ID NO: 504, 508, and 509, or an immunogenic fragment or immunogenic variant thereof.

Embodiment 23 (b). The coding RNA according to any one of the preceding embodiments, wherein the coding sequence encodes an amino acid sequence which is identical or at least 90%, 91%, 92%, 93%, 94%, 95%, 96%, 97%, 98%, or 99% identical to any one of SEQ ID NO: 504, or an immunogenic fragment or immunogenic variant thereof.

Embodiment 23 (c). The coding RNA according to any one of the preceding embodiments, wherein the coding sequence encodes an amino acid sequence which is identical or at least 90%, 91%, 92%, 93%, 94%, 95%, 96%, 97%, 98%, or 99% identical to any one of SEQ ID NO: 508, or an immunogenic fragment or immunogenic variant thereof.

Embodiment 23 (d). The coding RNA according to any one of the preceding embodiments, wherein the coding sequence encodes an amino acid sequence which is identical or at least 90%, 91%, 92%, 93%, 94%, 95%, 96%, 97%, 98%, or 99% identical to any one of SEQ ID NO: 509, or an immunogenic fragment or immunogenic variant thereof.

Embodiment 24. The coding RNA according to any one of the embodiments 1 to 22, wherein the coding sequence comprises a nucleic acid sequences which is identical or at least 70%, 80%, 85%, 86%, 87%, 88%, 89%, 90%, 91%, 92%, 93%, 94%, 95%, 96%, 97%, 98%, or 99% identical to the sequences according to any one of SEQ ID NOs: 187-246, 257-316, 523-545, 548-570, 573-595, 598-620, 623-645, 648-670, or a fragment or a variant thereof.

Embodiment 25. The coding RNA according to any one of the preceding embodiments, wherein the coding sequence is a codon modified coding sequence, wherein the amino acid sequence encoded by the at least one codon modified coding sequence is optionally not being modified compared to the amino acid sequence encoded by the corresponding wild type coding sequence, optionally wherein the at least one codon modified coding sequence is selected from C maximized coding sequence, CAI maximized coding sequence, human codon usage adapted coding sequence, G/C content modified coding sequence, and G/C optimized coding sequence, or any combination thereof.

Embodiment 26 (a). The coding RNA according to embodiment 25, wherein the coding sequence comprises at least one of the nucleic acid sequences being identical or at least 90%, 91%, 92%, 93%, 94%, 95%, 96%, 97%, 98%, or 99% identical to the sequences according to any one of SEQ ID NOs: 523-545, 548-570, 573-595, 598-620, 623-645, 648-670, or a fragment or variant thereof.

Embodiment 26 (b). The coding RNA according to any one of the preceding embodiments, wherein the coding sequence comprises at least one of the nucleic acid sequences being identical or at least 70%, 80%, 85%, 86%, 87%, 88%, 89%, 90%, 91%, 92%, 93%, 94%, 95%, 96%, 97%, 98%, or 99% identical to the sequences according to any one of SEQ ID NOs: 529, 533, 534, 554, 558, 559, 579, 583, 584, 604, 608, 609, 629, 633, 634, 654, 658, 659, or a fragment or variant thereof.

Embodiment 27. The coding RNA according to any one of the preceding embodiments wherein the coding sequence is G/C optimized coding sequence.

Embodiment 28. The coding RNA according to embodiment 27, wherein the coding sequence comprises at least one of the nucleic acid sequences being identical or at least 90%, 91%, 92%, 93%, 94%, 95%, 96%, 97%, 98%, or 99% identical to the sequences according to any one of SEQ ID NO: 523-545, 548-570, 648-670, or a fragment or variant thereof.

Embodiment 29. The coding RNA according to embodiment 27, wherein the coding sequence comprises at least one of the nucleic acid sequences being identical or at least 70%, 80%, 85%, 86%, 87%, 88%, 89%, 90%, 91%, 92%, 93%, 94%, 95%, 96%, 97%, 98%, or 99% identical to the sequences according to any one of SEQ ID NOs: 529, 533, 534, 554, 558, 559, 654, 658, 659, or a fragment or variant thereof.

Embodiment 30. The coding RNA according to any one of the preceding embodiments, wherein the at least one UTR is selected from at least one 5′-UTR and/or at least one 3′-UTR, optionally wherein the at least one UTR is selected from at least one heterologous 5′-UTR and/or at least one heterologous 3′-UTR.

Embodiment 31. The coding RNA according to embodiment 30, wherein the coding RNA comprises at least one 3′-UTR, wherein the at least one 3′-UTR comprises or consists of a nucleic acid sequence derived from a 3′-UTR of a gene selected from PSMB3, ALB7, alpha-globin, CASP1, COX6B1, GNAS, NDUFA1 and RPS9, or from a homolog, a fragment or a variant of any one of these genes.

Embodiment 32. The coding RNA according to embodiment 31, wherein the at least one heterologous 3′-UTR comprises or consists of a nucleic acid sequence being identical or at least 70%, 80%, 85%, 86%, 87%, 88%, 89%, 90%, 91%, 92%, 93%, 94%, 95%, 96%, 97%, 98%, or 99% identical to SEQ ID NOs: 67-90, 109-120, or a fragment or a variant thereof.

Embodiment 33. The coding RNA according to embodiment 31, wherein the coding RNA comprises a 3′-UTR derived or selected from a PSMB3 gene.

Embodiment 34. The coding RNA according to embodiment 33, wherein the 3′-UTR derived or selected from a PSMB3 gene comprises or consist of a nucleic acid sequence being identical or at least 70%, 80%, 85%, 86%, 87%, 88%, 89%, 90%, 91%, 92%, 93%, 94%, 95%, 96%, 97%, 98%, or 99% identical to any one of SEQ ID NO: 67, 68, 109-120, or a fragment or a variant thereof.

Embodiment 35. The coding RNA according to any one of embodiments 30 to 34, wherein the coding RNA comprises at least one 5′-UTR, wherein the at least one (heterologous) 5′-UTR comprises or consists of a nucleic acid sequence derived from a 5′-UTR of a gene selected from HSD17B4, RPL32, ASAH1, ATP5A1, MP68, NDUFA4, NOSIP, RPL31, SLC7A3, TUBB4B and UBQLN2, or from a homolog, a fragment or variant thereof.

Embodiment 36. The coding RNA according to embodiment 35, wherein at least one (heterologous) 5′-UTR derived or selected from HSD17B4, RPL32, ASAH1, ATP5A1, MP68, NDUFA4, NOSIP, RPL31, SLC7A3, TUBB4B, and UBQLN2 comprises or consist of a nucleic acid sequence being identical or at least 70%, 80%, 85%, 86%, 87%, 88%, 89%, 90%, 91%, 92%, 93%, 94%, 95%, 96%, 97%, 98%, or 99% identical to any one of SEQ ID NOs: 1-32, 65, 66, or a fragment or a variant thereof.

Embodiment 37. The coding RNA according to embodiments 35 or 36, wherein the at least one (heterologous) 5′-UTR is selected from HSD17B4, optionally wherein the 5′-UTR derived or selected from HSD17B4 comprises or consists of a nucleic acid sequence being identical or at least 70%, 80%, 85%, 86%, 87%, 88%, 89%, 90%, 91%, 92%, 93%, 94%, 95%, 96%, 97%, 98%, or 99% identical to any one of SEQ ID NOs: 1, 2, 65, 66, or a fragment or a variant thereof.

Embodiment 38. The coding RNA according to any one of embodiments 30 to 37, wherein the at least one (heterologous) 5′-UTR is selected from HSD17B4 and the at least one (heterologous) 3′-UTR is selected from PSMB3.

Embodiment 39. The coding RNA according to any one of the preceding embodiments, wherein the coding RNA of the invention is monocistronic.

Embodiment 40. The coding RNA according to any one of the preceding embodiments, comprising at least one poly(A) sequence, optionally wherein the at least one poly(A) sequence comprises about 40 to about 500 adenosine nucleotides, for example about 60 to about 250 adenosine nucleotides, for example about 60 to about 150 adenosine nucleotides.

Embodiment 41. The coding RNA according to embodiment 40, wherein the at least one poly(A) sequence comprises about 100 adenosine nucleotides.

Embodiment 42. The coding RNA according to embodiments 40 or 41, wherein the at least one poly(A) sequence is located directly at the 3′ terminus, optionally, wherein the 3′ terminal nucleotide is an adenosine.

Embodiment 43. The coding RNA according to any one of the preceding embodiments, comprising at least one poly(C) sequence and/or at least one miRNA binding site and/or histone-stem loop sequence.

Embodiment 44. The coding RNA according to embodiment 43, comprising at least one histone stem-loop.

Embodiment 45. The coding RNA according to embodiment 44, wherein the histone-stem loop sequence comprises or consists of a nucleic acid sequence identical or at least 70%, 80%, 90%, 91%, 92%, 93%, 94%, 95%, 96%, 97%, 98%, or 99% identical to any one of SEQ ID NOs: 136, 137 or a fragment or a variant thereof.

Embodiment 46. The coding RNA according to any one of the preceding embodiments, comprising at least one 3′-terminal sequence element, optionally wherein the 3′-terminal sequence element comprises or consists of an RNA sequence being identical or at least 70%, 80%, 90%, 91%, 92%, 93%, 94%, 95%, 96%, 97%, 98%, or 99% identical to any one of SEQ ID NOs: 138-172, or a fragment or variant thereof.

Embodiment 47. The coding RNA according to embodiment 47, comprising a 3′-terminal sequence element comprising or consisting of an RNA sequence being identical or at least 70%, 80%, 90%, 91%, 92%, 93%, 94%, 95%, 96%, 97%, 98%, or 99% identical to SEQ ID NO: 144, or a fragment or variant thereof.

Embodiment 48. The coding RNA according to any one of the preceding embodiments, comprising a 5′-terminal sequence element comprising or consisting of an RNA sequence being identical or at least 70%, 80%, 90%, 91%, 92%, 93%, 94%, 95%, 96%, 97%, 98%, or 99% identical to any one of SEQ ID NOs: 121-127, or a fragment or variant of these sequences.

Embodiment 49. The coding RNA according to embodiment 48, comprising a 5′-terminal sequence element comprising or consisting of an RNA sequence being identical or at least 70%, 80%, 90%, 91%, 92%, 93%, 94%, 95%, 96%, 97%, 98%, or 99% identical to SEQ ID NO: 122, or a fragment or variant thereof.

Embodiment 50. The coding RNA according to any one of the preceding embodiments, comprising a 5′-cap structure.

Embodiment 51. The coding RNA according to embodiment 50, wherein the 5′-cap structure is selected from a cap1 structure or a modified cap1 structure.

Embodiment 52. The coding RNA according to embodiments 50 or 51, wherein the 5′-cap structure has been added co-transcriptionally using tri-nucleotide cap analogue, in particular in an RNA in vitro transcription.

Embodiment 53. The coding RNA according to any one of embodiments 50 to 52, comprising a cap1 structure.

Embodiment 54. The coding RNA according to embodiment 53, wherein the cap1 structure is formed via co-transcriptional capping using tri-nucleotide cap analogues m7G(5′)ppp(5′)(2′OMeA)pG or m7G(5′)ppp(5′)(2′OMeG)pG.

Embodiment 55. The coding RNA according to embodiment 54, wherein the cap1 analogue is m7G(5′)ppp(5′)(2′OMeA)pG.

Embodiment 56. The coding RNA according to any one of the preceding embodiments, comprising at least one modified nucleotide, optionally selected from pseudouridine (ψ) or N1-methylpseudouridine (m1ψ).

Embodiment 57. The coding RNA according to embodiment 56, wherein the coding sequence comprises at least one modified nucleotide selected from pseudouridine (ψ) and N1-methylpseudouridine (m1ψ), optionally wherein essentially all uracil nucleotides are replaced by pseudouridine (ψ) nucleotides and/or N1-methylpseudouridine (m1ψ) nucleotides.

Embodiment 58. The coding RNA according to any one of the preceding embodiments, wherein the nucleic acid, comprises at least one modified nucleotide which is N1-methylpseudouridine (m1ψ).

Embodiment 59. The coding RNA according to embodiments 57 or 58, wherein essentially all uracil nucleotides are replaced N1-methylpseudouridine (m1ψ) nucleotides.

Embodiment 60. The coding RNA according to any one of the preceding embodiments, wherein the coding RNA is selected from an mRNA, a coding self-replicating RNA, a coding circular RNA, a coding viral RNA, or a coding replicon RNA.

Embodiment 61. The coding RNA according to embodiment 60, wherein the coding RNA is an mRNA.

Embodiment 62. The coding RNA according to any one of the preceding embodiments, wherein the coding RNA is an in vitro transcribed RNA, optionally wherein RNA in vitro transcription has been performed in the presence of a sequence optimized nucleotide mixture.

Embodiment 63. The coding RNA according to any one of the preceding embodiments, wherein the coding RNA is a purified RNA, optionally wherein the RNA has been purified by RP-HPLC, AEX, SEC, hydroxyapatite chromatography, TFF, filtration, precipitation, core-bead flow through chromatography, oligo(dT) purification, cellulose-based purification, or any combination thereof.

Embodiment 64. The coding RNA according to embodiment 63, wherein the RNA has been purified by RP-HPLC and/or TFF.

Embodiment 65. The coding RNA according to any one of the preceding embodiments, wherein the coding RNA has an integrity of at least about 50%, for example of at least about 60%, more for example of at least about 70%, for example of at least about 80%.

Embodiment 66. The coding RNA according to any one of the preceding embodiments, wherein comprising the following elements, for example in 5′ to 3′ direction:
A) a 5′-cap structure;
B) a 5′-UTR for example selected or derived from a 5′-UTR of a HSD17B4 gene;
C) at least one coding sequence encoding an antigenic polypeptide which is selected or derived from Escherichia coli FimH;
D) a 3′-UTR for example selected or derived from a 3′-UTR of a PSMB3 gene; E) optionally, a histone stem-loop; and
F) a poly(A) sequence for example comprising about 100 A nucleotides.

Embodiment 67. The coding RNA according to any one of the preceding embodiments, comprising or consisting of a nucleic acid sequence which is identical or at least 70%, 80%, 85%, 86%, 87%, 88%, 89%, 90%, 91%, 92%, 93%, 94%, 95%, 96%, 97%, 98%, or 99% identical to a nucleic acid sequence according to any one of SEQ ID NOs: 673-695, 698-720, 723-745, 748-770, 773-795, 798-820, 823-845, 848-870, 873-895, 898-920, 923-945, 948-970, 973-995, 998-1020, 1023-1045, 1048-1070, 1073-1095, 1098-1120, 1123-1145, 1148-1170, 1173-1195, 1198-1220, 1223-1245, 1248-1270 or a fragment or variant thereof.

Embodiment 68. The coding RNA according to embodiment 67, wherein at least one, for example all uracil nucleotides in said RNA sequences are replaced by pseudouridine (ψ) nucleotides and/or N1-methylpseudouridine (m1ψ) nucleotides.

Embodiment 69. The coding RNA according to any one of the preceding embodiments, wherein the coding RNA comprises or consists of a nucleic acid sequence which is identical or at least 70%, 80%, 85%, 86%, 87%, 88%, 89%, 90%, 91%, 92%, 93%, 94%, 95%, 96%, 97%, 98%, or 99% identical to a nucleic acid sequence according to any one of SEQ ID NOs: 679, 704, 729, 754, 779, 804, 829, 854, 879, 904, 929, 954, 979, 1004, 1029, 1054, 1079, 1104, 1129, 1154, 1179, 1204, 1229, 1254 or a fragment or variant thereof, optionally comprising a 5′-terminal cap1 structure.

Embodiment 70. The coding RNA according to any one of the preceding embodiments, wherein the coding RNA comprises or consists of a nucleic acid sequence which is identical or at least 70%, 80%, 85%, 86%, 87%, 88%, 89%, 90%, 91%, 92%, 93%, 94%, 95%, 96%, 97%, 98%, or 99% identical to a nucleic acid sequence according to any one of SEQ ID NOs: 683, 708, 733, 758, 783, 808, 833, 858, 883, 908, 933, 958, 983, 1008, 1033, 1058, 1083, 1108, 1133, 1158, 1183, 1208, 1233, 1258 or a fragment or variant thereof, optionally comprising a 5′-terminal cap1 structure.

Embodiment 71. The coding RNA according to any one of the preceding embodiments, wherein In one embodiment, the coding RNA comprises or consists of a nucleic acid sequence which is identical or at least 70%, 80%, 85%, 86%, 87%, 88%, 89%, 90%, 91%, 92%, 93%, 94%, 95%, 96%, 97%, 98%, or 99% identical to a nucleic acid sequence according to any one of SEQ ID NOs: 684, 709, 734, 759, 784, 809, 834, 859, 884, 909, 934, 959, 984, 1009, 1034, 1059, 1084, 1109, 1134, 1159, 1184, 1209, 1234, 1259 or a fragment or variant thereof, optionally comprising a 5′-terminal cap1 structure.

Embodiment 72 (a). The coding RNA according to embodiment 69, wherein the coding RNA is a 5′ capped (cap1) mRNA that comprises or consists of an RNA sequence consisting of: non-modified ribonucleotides (A, G, C, U); non-modified ribonucleotides (A, G, C) and chemically modified pseudouridine (ψ) ribonucleotides; or non-modified ribonucleotides (A, G, C) and chemically modified N1-methylpseudouridine (m1ψ) ribonucleotides;
which is identical to an RNA sequence according to any one of SEQ ID NOs: 679, 829, 979, 1129, 1271, 1274 or a fragment or variant thereof.

Embodiment 72 (b). The coding RNA according to embodiment 69, wherein the coding RNA is a 5′ capped (cap1) mRNA that comprises or consists of an RNA sequence consisting of: non-modified ribonucleotides (A, G, C) and chemically modified N1-methylpseudouridine (m1ψ) ribonucleotides;
which is identical to an RNA sequence according to SEQ ID NO: 1271, or a fragment or variant thereof.

Embodiment 72 (c). The coding RNA according to embodiment 69, wherein the coding RNA is a 5′ capped (cap1) mRNA that comprises or consists of an RNA sequence consisting of: non-modified ribonucleotides (A, G, C) and chemically modified pseudouridine (ψ) ribonucleotides; which is identical to an RNA sequence according to SEQ ID NO: 1274 or a fragment or variant thereof.

Embodiment 73 (a). The coding RNA according to embodiment 70, wherein the coding RNA is a 5′ capped (cap1) mRNA that comprises or consists of an RNA sequence consisting of: non-modified ribonucleotides (A, G, C, U); non-modified ribonucleotides (A, G, C) and chemically modified pseudouridine (ψ) ribonucleotides; or non-modified ribonucleotides (A, G, C) and chemically modified N1-methylpseudouridine (m1ψ) ribonucleotides;
which is identical to an RNA sequence according to any one of SEQ ID NOs: 683, 833, 983, 1133, 1272, 1275 or a fragment or variant thereof.

Embodiment 73 (b). The coding RNA according to embodiment 70, wherein the coding RNA is a 5′ capped (cap1) mRNA that comprises or consists of an RNA sequence consisting of: non-modified ribonucleotides (A, G, C) and chemically modified N1-methylpseudouridine (m1ψ) ribonucleotides;
which is identical to an RNA sequence according to SEQ ID NO: 1272, or a fragment or variant thereof.

Embodiment 73 (c). The coding RNA according to embodiment 70, wherein the coding RNA is a 5′ capped (cap1) mRNA that comprises or consists of an RNA sequence consisting of: non-modified ribonucleotides (A, G, C) and chemically modified pseudouridine (ψ) ribonucleotides; which is identical to an RNA sequence according to SEQ ID NO: 1275 or a fragment or variant thereof.

Embodiment 74 (a). The coding RNA according to embodiment 71, wherein the coding RNA is a 5′ capped (cap1) mRNA that comprises or consists of an RNA sequence consisting of: non-modified ribonucleotides (A, G, C, U); non-modified ribonucleotides (A, G, C) and chemically modified pseudouridine (ψ) ribonucleotides; or non-modified ribonucleotides (A, G, C) and chemically modified N1-methylpseudouridine (m1ψ) ribonucleotides;
which is identical to an RNA sequence according to any one of SEQ ID NOs: 684, 834, 984, 1134, 1273, 1276 or a fragment or variant thereof.

Embodiment 74 (b). The coding RNA according to embodiment 69, wherein the coding RNA is a 5′ capped (cap1) mRNA that comprises or consists of an RNA sequence consisting of: non-modified ribonucleotides (A, G, C) and chemically modified N1-methylpseudouridine (m1ψ) ribonucleotides;
which is identical to an RNA sequence according to SEQ ID NO: 1273, or a fragment or variant thereof.

Embodiment 74 (c). The coding RNA according to embodiment 69, wherein the coding RNA is a 5′ capped (cap1) mRNA that comprises or consists of an RNA sequence consisting of: non-modified ribonucleotides (A, G, C) and chemically modified pseudouridine (ψ) ribonucleotides; which is identical to an RNA sequence according to SEQ ID NO: 1276 or a fragment or variant thereof.

Embodiment 75. A pharmaceutical composition comprising the coding RNA according to any one of the preceding embodiments.

Embodiment 76. The pharmaceutical composition according to embodiment 75, comprising at least one pharmaceutically acceptable carrier or excipient.

Embodiment 77. The pharmaceutical composition according to embodiments 75 or 76, comprising a lipid-based carrier, optionally wherein the coding RNA is formulated in the lipid-based carrier.

Embodiment 78. The pharmaceutical composition according to embodiment 77, wherein the lipid-based carrier is selected from liposomes, lipid nanoparticles, lipoplexes, solid lipid nanoparticles, lipo-polylexes, and/or nanoliposomes.

Embodiment 79. The pharmaceutical composition according to embodiment 78, wherein the lipid-based carrier is a lipid nanoparticle, optionally wherein the lipid nanoparticle encapsulates the coding RNA.

Embodiment 80. The pharmaceutical composition according to any one of embodiments 75 to 79, wherein the coding RNA is formulated in at least one cationic or polycationic compound.

Embodiment 81. The pharmaceutical composition according to embodiment 80, wherein the at least one cationic or polycationic compound is selected from a cationic or polycationic polymer, a cationic or polycationic polysaccharide, a cationic or polycationic lipid, a cationic or polycationic protein, a cationic or polycationic peptide, or any combinations thereof.

Embodiment 82. The pharmaceutical composition according to any one of embodiments 77 to 81, wherein the lipid-based carrier comprises at least one lipid selected from an aggregation-reducing lipid, a cationic lipid or ionizable lipid, a neutral lipid or phospholipid, or a steroid, steroid analogue or sterol, or any combinations thereof.

Embodiment 83. The pharmaceutical composition according to embodiment 82, the lipid-based carrier comprises an aggregation-reducing lipid, a cationic lipid or ionizable lipid, a neutral lipid or phospholipid, and a steroid, steroid analogue or sterol.

Embodiment 84. The pharmaceutical composition according to any one of embodiments 77 to 83, wherein the lipid-based carrier comprises a cationic lipid selected or derived from Formula III, for example formula III-3.

Embodiment 85. The pharmaceutical composition according to any one of embodiments 77 to 84, wherein the lipid-based carrier comprises a cationic lipid selected or derived from ALC-0315.

Embodiment 86. The pharmaceutical composition according to any one of embodiments 77 to 85, wherein the lipid-based carrier comprises an aggregation reducing lipid selected from a polymer conjugated lipid, optionally wherein the polymer conjugated lipid is a PEG-conjugated lipid selected or derived from Formula IVa, for example selected or derived from ALC-0159.

Embodiment 87. The pharmaceutical composition according to any one of embodiments 77 to 86, wherein the lipid-based carrier comprises a neutral lipid selected or derived from DSPC.

Embodiment 88. The pharmaceutical composition according to any one of embodiments 77 to 87, wherein the lipid-based carrier comprises a steroid, steroid analogue or sterol, which is optionally selected or derived from cholesterol.

Embodiment 89. The pharmaceutical composition according to any one of embodiments 77 to 88, wherein the lipid-based carrier comprises
(i) at least one cationic lipid, for example according to embodiment 84 or 85;
(ii) at least one neutral lipid, for example according to embodiment 87;
(iii) at least one steroid, steroid analogue or sterol, for example according to embodiment 88; and
(iv) at least one aggregation reducing lipid, for example according to embodiment 86.

Embodiment 90. The pharmaceutical composition according to any one of embodiments 77 to 89, wherein the lipid-based carrier comprises
(i) at least one cationic lipid selected from ALC-0315;
(ii) at least one neutral lipid selected from DSPC;
(iii) at least one steroid, steroid analogue or sterol selected from cholesterol; and
(iv) at least one aggregation reducing lipid selected from ALC-0159.

Embodiment 91. The pharmaceutical composition according to any one of embodiments 89 to 90, wherein the lipid base carrier comprises (i) to (iv) in a molar ratio of about 20-60% cationic lipid or ionizable lipid, about 5-25% neutral lipid, about 25-55% steroid or steroid analogue, and about 0.5-15% aggregation reducing lipid Embodiment 92. The pharmaceutical composition according to any one of embodiments 77 to 91, wherein the wt/wt ratio of lipid to coding RNA in the lipid-based carrier is from about 10:1 to about 60:1, for example from about 20:1 to about 30:1.

Embodiment 93. The pharmaceutical composition according to any one of embodiments 77 to 92, wherein the N/P ratio of the lipid-based carrier encapsulating the nucleic acid is in a range from about 1 to about 10, for example in a range from about 5 to about 7.

Embodiment 94. The pharmaceutical composition according to any one of embodiments 77 to 93, wherein the lipid-based carrier has a Z-average size in a range of about 50 nm to about 120 nm.

Embodiment 95. The pharmaceutical composition according to any one of embodiments 75 to 94, additionally comprising at least one antagonist of at least one RNA sensing pattern recognition receptor selected from a Toll-like receptor, for example a TLR7 antagonist and/or a TLR8 antagonist.

Embodiment 96. The pharmaceutical composition according to any one of embodiments 75 to 95, wherein the composition is a liquid composition or a dried composition.

Embodiment 97. A vaccine comprising the coding RNA according to any one of embodiments 1 to 74, or the pharmaceutical composition according to any one of embodiments 75 to 96.

Embodiment 98. The vaccine according to embodiment 97, wherein the vaccine, for example the administration of the vaccine to a subject, elicits a humoral immune response against E. coli, FimH.

Embodiment 99. The vaccine according to embodiments 97 or 98, wherein the vaccine, for example the administration of the vaccine to a subject, elicits neutralizing antibody titers against E. coli, optionally wherein said antibodies are IgG antibodies. In one embodiment, said antibodies are against E. coli FimH. In one embodiment, the vaccine, for example the administration of the vaccine to a subject, elicits neutralizing antibody titers against E. coli, for example against E. coli FimH, in the urine of a subject upon administration of the vaccine.

Embodiment 100. A Kit or kit of parts, comprising the coding RNA according to any one of embodiments 1 to 74, the pharmaceutical composition according to any one of embodiments 75 to 96, and/or the vaccine according to any one of embodiments 97 to 99, optionally comprising a liquid vehicle for solubilising, and, optionally, technical instructions providing information on administration and dosage of the components.

Embodiment 101. The coding RNA according to any one of embodiments 1 to 74, the pharmaceutical composition according to any one of embodiments 75 to 96, the vaccine according to any one of embodiments 97 to 99, or the kit or kit of parts according to embodiment 100, for use as a medicament.

Embodiment 102. The coding RNA according to any one of embodiments 1 to 74, the pharmaceutical composition according to any one of embodiments 75 to 96, the vaccine according to any one of embodiments 97 to 99, or the kit or kit of parts according to embodiment 100, for use for treating or preventing one or more symptoms associated with urinary tract infections (UTI) in a subject in need thereof.

Embodiment 103. The coding RNA according to any one of embodiments 1 to 74, the pharmaceutical composition according to any one of embodiments 75 to 96, the vaccine according to any one of embodiments 97 to 99, or the kit or kit of parts according to embodiment 100, for use for treating or preventing a disease caused by E. coli.

Embodiment 104. A method of treating or preventing a disorder, wherein the method comprises administering to a subject in need thereof an effective amount of the coding RNA according to any one of embodiments 1 to 74, the pharmaceutical composition according to any one of embodiments 75 to 96, the vaccine according to any one of embodiments 97 to 99, or the kit or kit of parts according to embodiment 100.

Embodiment 105. The method according to embodiment 104, wherein the method elicits a humoral immune response against E. coli FimH, optionally wherein the method elicits a humoral immune response in the urine of the subject.

Embodiment 106. The method according to embodiments 104 or 105, wherein the method elicits neutralizing antibody titers against E. coli, optionally wherein said antibodies are IgG antibodies.

Embodiment 107. The method according to embodiment 106, wherein the method elicits neutralizing antibody titers against E. coli, for example against E. coli FimH, in the urine of the subject.

Embodiment 108. The method according to any one of embodiments 104 to 107, wherein the method elicits antibodies which are capable of inhibiting bacterial adhesion.

Embodiment 109. The method according to any one of embodiments 104 to 108, wherein the administration is an intramuscular administration.

Embodiment 110. Use of the coding RNA according to any one of embodiments 1 to 74, the pharmaceutical composition according to any one of embodiments 75 to 96, the vaccine according to any one of embodiments 97 to 99, or the kit or kit of parts according to embodiment 100 for the manufacture of a medicament for raising an immune response in a mammal, for example, for treating and/or preventing a disease, for example an E. coli infection.

### EXAMPLES

In the following, particular examples illustrating various embodiments and aspects of the disclosure are presented. However, the present disclosure shall not to be limited in scope by the specific embodiments described herein. The following preparations and examples are given to enable those skilled in the art to more clearly understand and to practice the present disclosure. The present disclosure, however, is not limited in scope by the exemplified embodiments, which are intended as illustrations of single aspects of the disclosure only, and methods, which are functionally equivalent are within the scope of the disclosure. Indeed, various modifications of the disclosure in addition to those described herein will become readily apparent to those skilled in the art from the foregoing description, accompanying figures and the examples below. All such modifications fall within the scope of the appended claims.

### Example 1: Preparation of DNA and RNA Constructs, Compositions, and Vaccines

The present Example provides methods of obtaining the coding RNA of the disclosure as well as methods of generating a composition or a vaccine of the disclosure.

### 1.1. Preparation of DNA and RNA Constructs

DNA sequences encoding different E. coli FimH protein designs were prepared and used for subsequent RNA in vitro transcription reactions. Said DNA sequences were prepared by modifying the wild type or reference encoding DNA sequences by introducing a G/C optimized or modified coding sequence (e.g., “cds opt1”) for stabilization and expression optimization. Sequences were introduced into a pUC derived DNA vector to comprise stabilizing 3′-UTR sequences and 5′-UTR sequences, additionally comprising a stretch of adenosines (e.g. A100), and optionally a histone stem-loop (hSL) structure (see Table 3, for an overview of antigen designs see Table 1).

The obtained plasmid DNA constructs were transformed and propagated in bacteria using common protocols known in the art. Eventually, the plasmid DNA constructs were extracted, purified, and used for subsequent RNA in vitro transcription (see section 1.2.).

1.2. RNA In Vitro Transcription from Plasmid DNA Templates

DNA plasmids prepared according to section 1.1 were enzymatically linearized using a restriction enzyme and used for DNA dependent RNA in vitro transcription using T7 RNA polymerase in the presence of a sequence optimized nucleotide mixture (ATP/GTP/CTP/UTP) and cap analog (for cap1: m7G(5′)ppp(5′)(2′OMeA)pG; TriLink) under suitable buffer conditions. The obtained RNA constructs were purified using RP-HPLC (PureMessenger®, CureVac AG, Tübingen, Germany; WO2008077592) and used for in vitro and in vivo experiments. To obtain chemically modified mRNA, RNA in vitro transcription was performed in the presence of a modified nucleoside mixture comprising N1-methylpseudouridine (m1ψ) or pseudouridine (ψ) instead of uridine. The obtained m1ψ or ψ chemically modified RNA was purified using RP-HPLC (PureMessenger®, CureVac AG, Tübingen, Germany; WO2008077592) and used for further experiments.

RNA for clinical development is produced under current good manufacturing practice e.g. according to WO2016180430, implementing various quality control steps on DNA and RNA level.

### The RNA Constructs of the Examples

The generated RNA sequences/constructs are provided in Table 3 with the encoded antigenic protein and the respective UTR elements indicated therein. If not indicated otherwise, the RNA sequences/constructs of Table 3 have been produced using RNA in vitro transcription in the presence of a m7G(5′)ppp(5′)(2′OMeA)pG cap analog; accordingly, the RNA sequences/constructs comprise a 5′ cap1 structure. If not indicated otherwise, the RNA sequences/constructs of Table 3 have been produced in the absence of chemically modified nucleotides (e.g. pseudouridine (ψ) or N1-methylpseudouridine (m1ψ)).

TABLE 3
RNA constructs encoding the antigen designs used in the examples

| Construct | | | 5′-UTR/ | | SEQ | | |
|---|---|---|---|---|---|---|---|
| # (RNA | | CDS | 3′-UTR; | 3′- | ID NO: | SEQ ID | SEQ ID |
| ID) | Description | opt. | UTR Design | end | Protein | NO: CDS | NO: RNA |
| 1 | Ec(J96)-FimC(1- | opt1 | HSD17B4/ | hSL- | 496 | 521 | 671 |
| (R10388) | 36)_Ec(J96)-FimC(37-241) | (gc) | PSMB3; | A100 | | | |
| | | | a-1 | | | | |
| 2 | HsIgE(1-18)_Ec(J96)- | opt1 | HSD17B4/ | hSL- | 497 | 522 | 672 |
| (R10389) | FimC(37-241) | (gc) | PSMB3; | A100 | | | |
| | | | a-1 | | | | |
| 3 | Ec(J96)-FimH(1- | opt1 | HSD17B4/ | hSL- | 498 | 523 | 673 |
| (R10390) | 21)_Ec(J96)-FimH(22-300) | (gc) | PSMB3; | A100 | | | |
| | | | a-1 | | | | |
| 4 | HsIgE(1-18)_Ec(J96)- | opt1 | HSD17B4/ | hSL- | 499 | 524 | 674 |
| (R10391) | FimH(22-300) | (gc) | PSMB3; | A100 | | | |
| | | | a-1 | | | | |
| 5 | MmIgK(1-20)_Ec(J96)- | opt1 | HSD17B4/ | hSL- | 500 | 525 | 675 |
| (R10392) | FimH(22-300) | (gc) | PSMB3; | A100 | | | |
| | | | a-1 | | | | |
| 6 | Ec(J96)-FimH(1- | opt1 | HSD17B4/ | hSL- | 501 | 526 | 676 |
| (R10393) | 21)_Ec(J96)-FimH(22- | (gc) | PSMB3; | A100 | | | |
| | 300)_Linker- | | a-1 | | | | |
| | PGDGN_Ec(J96)-FimG(24- | | | | | | |
| | 37) | | | | | | |
| 7 | HsIgE(1-18)_Ec(J96)- | opt1 | HSD17B4/ | hSL- | 502 | 527 | 677 |
| (R10394) | FimH(22-300)_Linker- | (gc) | PSMB3; | A100 | | | |
| | PGDGN_Ec(J96)-FimG(24- | | a-1 | | | | |
| | 37) | | | | | | |
| 8 | HsIgE(1-18)_Ec(J96)- | opt1 | HSD17B4/ | hSL- | 503 | 528 | 678 |
| (R10395) | FimH(22-300)_Linker- | (gc) | PSMB3; | A100 | | | |
| | G4SGG_Ec(J96)-FimG(24- | | a-1 | | | | |
| | 37) | | | | | | |
| 9 | MmIgK(1- | opt1 | HSD17B4/ | hSL- | 504 | 529 | 679 |
| (R10396) | 20)_Ec(J96)FimH(22- | (gc) | PSMB3; | A100 | | | |
| | 300)_Linker- | | a-1 | | | | |
| | PGDGN_Ec(J96)-FimG(24- | | | | | | |
| | 37) | | | | | | |
| 9 | MmIgK(1- | opt1 | HSD17B4/ | hSL- | 504 | 529 | 829, |
| (R10949*, | 20)_Ec(J96)FimH(22- | (gc) | PSMB3; | A100 | | | 1271, |
| R10950*) | 300)_Linker- | | a-1 | | | | 1274 |
| | PGDGN_Ec(J96)-FimG(24- | | | | | | |
| | 37) | | | | | | |
| 10 | MmIgK(1- | opt1 | HSD17B4/ | hSL- | 505 | 530 | 680 |
| (R10397) | 20)_Ec(536)FimH(22- | (gc) | PSMB3; | A100 | | | |
| | 300)_Linker- | | a-1 | | | | |
| | PGDGN_Ec(J96)-FimG(24- | | | | | | |
| | 37) | | | | | | |
| 11 | HsIgE(1- | opt1 | HSD17B4/ | hSL- | 506 | 531 | 681 |
| (R10398) | 18)_Ec(J96)FimH(22- | (gc) | PSMB3; | A100 | | | |
| | 300)_Linker- | | a-1 | | | | |
| | (G4S)3_Ec(J96)-FimC(135- | | | | | | |
| | 148) | | | | | | |
| 12 | LumSynth(1- | opt1 | HSD17B4/ | hSL- | 507 | 532 | 682 |
| (R10399) | 154_C37A_N102D)_Linker- | (gc) | PSMB3; | A100 | | | |
| | (GGS)4-GGG_Ec(J96)- | | a-1 | | | | |
| | FimH(22-300)_Linker- | | | | | | |
| | PGDGN_Ec(J96)-FimG(24- | | | | | | |
| | 37) | | | | | | |
| 13 | HsIgE(1-18)_Ec(J96)- | opt1 | HSD17B4/ | hSL- | 508 | 533 | 683 |
| (R10400) | FimH(22-300)_Linker- | (gc) | PSMB3; | A100 | | | |
| | PGDGN_Ec(J96)-FimG(24- | | a-1 | | | | |
| | 37)_Linker- | | | | | | |
| | SGG_LumSynth(1- | | | | | | |
| | 154_C37A_N102D) | | | | | | |
| 13 | HsIgE(1-18)_Ec(J96)- | opt1 | HSD17B4/ | hSL- | 508 | 533 | 833, |
| (R10951*, | FimH(22-300)_Linker- | (gc) | PSMB3; | A100 | | | 1272, |
| R10952*) | PGDGN_Ec(J96)-FimG(24- | | a-1 | | | | 1275 |
| | 37)_Linker- | | | | | | |
| | SGG_LumSynth(1- | | | | | | |
| | 154_C37A_N102D) | | | | | | |
| 14 | HsIgE(1-18)_Ec(J96)- | opt1 | HSD17B4/ | hSL- | 509 | 534 | 684 |
| (R10401) | FimH(22-300)_Linker- | (gc) | PSMB3; | A100 | | | |
| | PGDGN_Ec(J96)-FimG(24- | | a-1 | | | | |
| | 37)_Linker-SGG_Ferritin(5- | | | | | | |
| | 167_N19Q) | | | | | | |
| 14 | HsIgE(1-18)_Ec(J96)- | opt1 | HSD17B4/ | hSL- | 509 | 534 | 834, |
| (R10953*, | FimH(22-300)_Linker- | (gc) | PSMB3; | A100 | | | 1273, |
| R10954*) | PGDGN_Ec(J96)-FimG(24- | | a-1 | | | | 1276 |
| | 37)_Linker-SGG_Ferritin(5- | | | | | | |
| | 167_N19Q) | | | | | | |
| 15 | MmIgK(1-20)_Ec(J96)- | opt1 | HSD17B4/ | hSL- | 510 | 535 | 685 |
| (R10402) | FimH(22-300)_Linker- | (gc) | PSMB3; | A100 | | | |
| | PGDGN_Ec(J96)-FimG(24- | | a-1 | | | | |
| | 37)_Linker- | | | | | | |
| | (GGS)4_Ferritin(5-166) | | | | | | |
| 16 | HsIgE(1-18)_Ec(J96)- | opt1 | HSD17B4/ | hSL- | 511 | 536 | 686 |
| (R10403) | FimH(22-300)_Linker- | (gc) | PSMB3; | A100 | | | |
| | PGDGN_Ec(J96)-FimG(24- | | a-1 | | | | |
| | 37)_TMdomain-HA(510- | | | | | | |
| | 566) | | | | | | |

*mRNA R10949, R10951 and R10953 were produced with pseudouridine (ψ); R10950, R10952 and R10954 were produced with N1-methylpseudouridine (m1ψ).
Ec: Escherichia coli; HA: Hemagglutinin; Hs: Homo sapiens; IgE: immunoglobulin E; IgK: immunoglobulin Kappa; LumSynth, LS: Lumazine synthase; Mm: Mus musculus; TMdomain, TM: transmembrane domain.

1.4. Preparation of a LNP Formulated mRNA Composition

LNPs were prepared using cationic lipids, structural lipids, a PEG-lipid, and cholesterol. Lipid solution (in ethanol) was mixed with RNA solution (aqueous buffer) using a microfluidic mixing device. Obtained LNPs were re-buffered in a carbohydrate buffer via dialysis, and up-concentrated to a target concentration using ultracentrifugation tubes. LNP-formulated mRNA was stored at −80° C. prior to use in in vitro or in vivo experiments.

Suitably, lipid nanoparticles were prepared and tested according to the general procedures described in POT Pub. Nos. WO2015199952, WO2017004143 and WO2017075531, the full disclosures of which are incorporated herein by reference. Lipid nanoparticle (LNP)-formulated mRNA was prepared using an ionizable amino lipid (cationic lipid), phospholipid, cholesterol and a PEGylated lipid. LNPs were prepared as follows. Cationic lipid according to formula III-3 (ALC-0315), DSPC, cholesterol and PEG-lipid according to formula IVa (ALC-0159) were solubilized in ethanol at a molar ratio of approximately 47.5:10:40.8:1.7 (see Table 4). Lipid nanoparticles (LNP) comprising compound Rai-3 were prepared at a ratio of mRNA (sequences see Table 3) to total lipid of 0.03-0.04 w/w. Briefly, the mRNA was diluted to 0.05 to 0.2 mg/ml in 10 to 50 mM citrate buffer, pH 4. Pumps were used to mix the ethanolic lipid solution with the mRNA aqueous solution at a ratio of about 1:5 to 1:3 (vol/vol) with total flow rates above 15 ml/min. The ethanol was then removed and the external buffer replaced with PBS. Finally, the lipid nanoparticles were filtered through a 0.2 μm pore sterile filter. Lipid nanoparticle particle diameter size was 50-120 nm as determined by quasi-elastic light scattering using a Malvern Zetasizer Nano (Malvern, UK).

TABLE 4
Lipid-based carrier composition of the examples

| | | Ratio | | |
|---|---|---|---|---|
| | Compounds | (mol %) | Structure | Mass |
| 1 | Cholesterol | 40.9 | 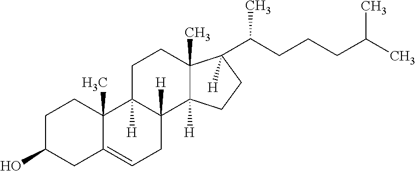 | 386.4 |
| 2 | 1,2-distearoylsn- glycero-3- phosphocholine (DSPC) | 10 | 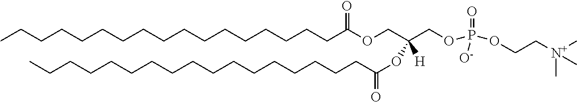 [SMILES: CCCCCCCCCCCCCCCCCC(=O)OC[C@H](COP(=O)([O-])OCC[N+](C)(C)C)OC(=O)CCCCCCCCCCCCCCCCC] | 789.6 |
| 3 | Cationic Lipid | 47.4 | 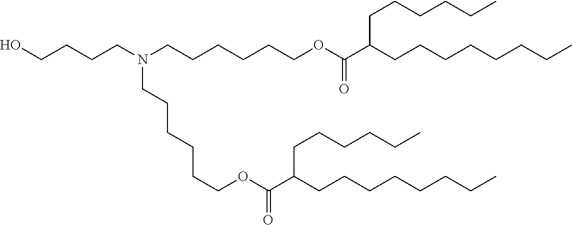 [SMILES: CCCCCCCCC(CCCCCC)C(=O)OCCCCCCN(CCCCO)CCCCCCOC(=O)C(CCCCCC)CCCCCCCC] | 765.7 |
| 4 | PEG Lipid | 1.7 | 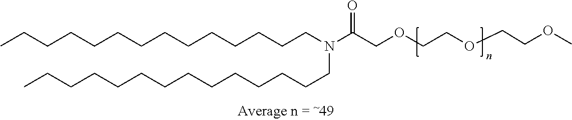 | 2010.1 |

1.5. Preparation of Combination mRNA Vaccines Comprising Antigen Combinations (Bivalent or Multivalent Vaccine Compositions):

Combination mRNA vaccines were formulated with LNPs either in a separate or co-formulated way. For separately mixed or formulated mRNA vaccines, each mRNA component was prepared and separately LNP formulated as described in Example 1.4, followed by mixing of the different LNP-formulated components. For co-formulated mRNA vaccine, the different mRNA components were firstly mixed together, followed by a co-formulation in LNPs as described in Example 1.4.

### Example 2: Analysis of E. coli FimH Antigen Designs

### 2.1 In Vitro Analysis of Expression and Secretion of Antigen Designs Using Western Blot

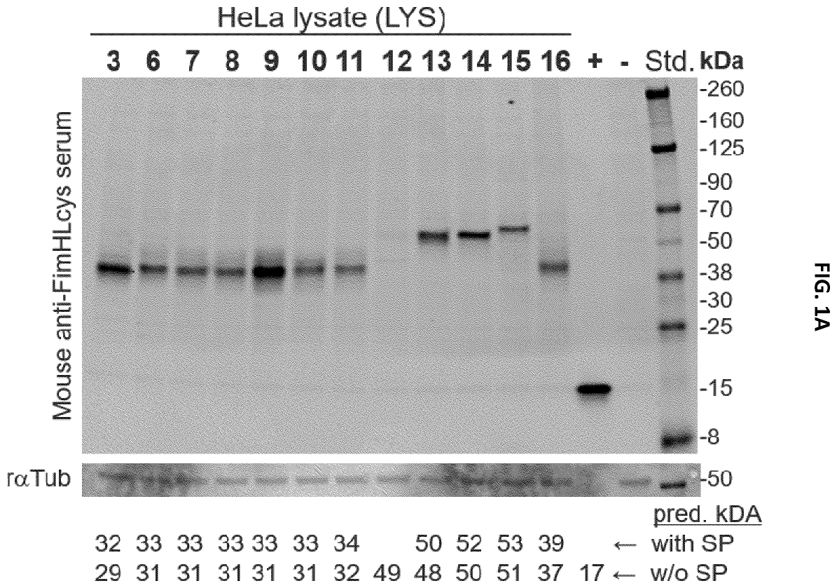

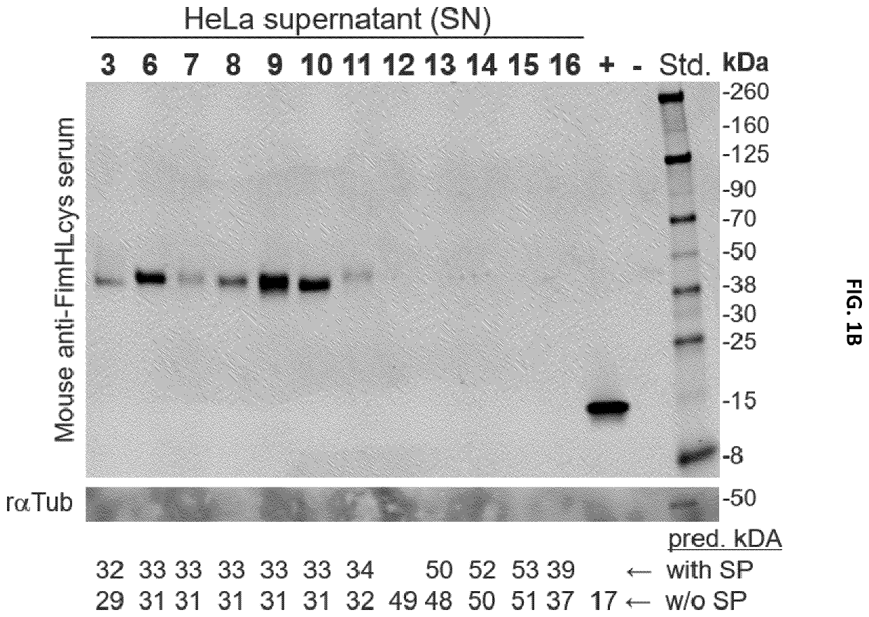

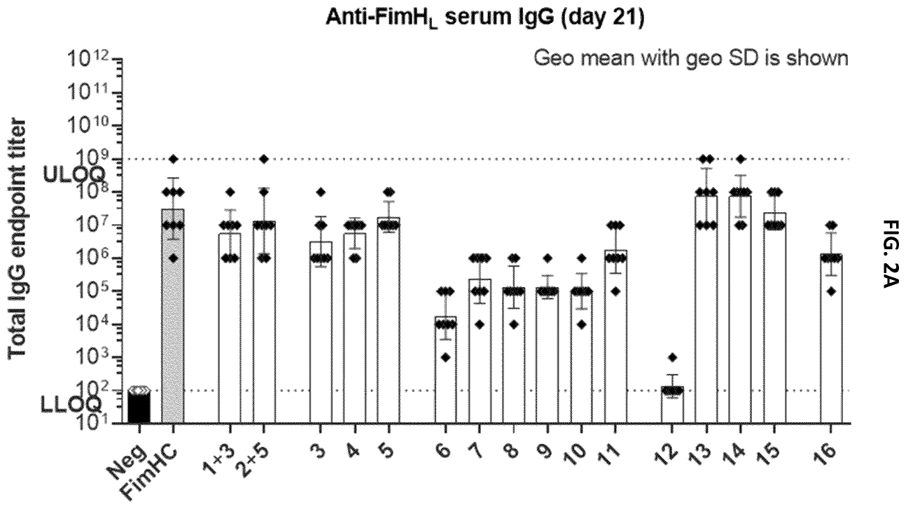

To determine the in vitro protein expression of some of the mRNA constructs, HeLa cells were transfected with 2 μg unformulated mRNA encoding different antigen designs using Lipofectamine 2000 and 6-well plates. 24 h after transfection, cell lysates and cell culture supernatants were subjected to SDS-PAGE and Western blot analysis using mouse anti-FimC serum (1:1000), mouse anti-FimHLcys serum (1:1000) or rabbit anti-alpha-tubulin antibody (1:1000; Cell Signaling) as primary antibodies as well as goat anti-rabbit IgG IRDye®680RD antibody (1:10000; Li-Cor) or goat anti-mouse IgG IRDye® 800CW antibody (1:10000; Li-Cor) as secondary antibodies. Anti-FimC serum and anti-FimHLcys sera were obtained by immunizing CD1 mice subcutaneously on days 0, 21 and 35 and collecting sera at day 49. FimHLcys was obtained as described in Kisiela D I, et al. Proc Natl Acad Sci USA. 2013 Nov. 19; 110(47):19089-94. Detection and quantification was performed using a Li-Cor detection system (Odyssey CLx image system) in combination with Image Studio Lite software. Table 5 contains mRNA constructs that were used in the experiment and the result of the experiment is shown in FIG. 1.

TABLE 5
mRNA constructs used for Western blot analysis (Example 2.1)

| | | | Pre- | |
|---|---|---|---|---|
| | | | dicted | |
| | | Predicted | size in | |
| | | size in | kDa | SEQ |
| Construct | | kDa with | w/o | ID |
| # | | signal | signal | NO: |
| (RNA ID) | Antigen design | peptide | peptide | RNA |
| Negative | WFI (water for injection) | — | — | — |
| control | | | | |
| Protein | Recombinant FimC | 22.7 (FimC) | | — |
| | or FimHL protein | 16.9 (FimHL) | | |
| | (100 ng loaded on | | | |
| | each gel) | | | |
| 1 | Ec(J96)-FimC(1-36)_Ec(J96)- | 26.6 | 22.7 | 671 |
| (R10388) | FimC(37-241) | | | |
| 2 | HslgE(1-18)_Ec(J96)- | 24.8 | 22.7 | 672 |
| (R10389) | FimC(37-241) | | | |
| 3 | Ec(J96)-FimH(1-21)_Ec(J96)- | 31.5 | 29.1 | 673 |
| (R10390) | FimH(22-300) | | | |
| 4 | HslgE(1-18)_Ec(J96)- | 31.2 | 29.1 | 674 |
| (R10391) | FimH(22-300) | | | |
| 5 | MmlgK(1-20)_Ec(J96)- | 31.3 | 29.1 | 675 |
| (R10392) | FimH(22-300) | | | |
| 6 | Ec(J96)-FimH(1-21)_Ec(J96)- | 33.3 | 30.9 | 676 |
| (R10393) | FimH(22-300)_Linker- | | | |
| | PGDGN_Ec(J96)-FimG(24-37) | | | |
| 7 | HslgE(1-18)_Ec(J96)-FimH(22- | 33.0 | 30.9 | 677 |
| (R10394) | 300)_Linker-PGDGN_Ec(J96)- | | | |
| | FimG(24-37) | | | |
| 8 | HslgE(1-18)_Ec(J96)-FimH(22- | 33.0 | 30.9 | 678 |
| (R10395) | 300)_Linker-G4SGG_Ec(J96)- | | | |
| | FimG(24-37) | | | |
| 9 | MmlgK(1-20)_Ec(J96)- | 33.1 | 30.9 | 679 |
| (R10396) | FimH(22-300)_Linker- | | | |
| | PGDGN_Ec(J96)- | | | |
| | FimG(24-37) | | | |
| 10 | MmlgK(1-20)_Ec(536)- | 33.1 | 30.9 | 680 |
| (R10397) | FimH(22-300)_Linker- | | | |
| | PGDGN_Ec(J96)- | | | |
| | FimG(24-37) | | | |
| 11 | HslgE(1-18)_Ec(J96)- | 33.7 | 31.6 | 681 |
| (R10398) | FimH(22-300)_Linker- | | | |
| | (G4S)3_Ec(J96)- | | | |
| | FimC(135-148) | | | |
| 12 | LumSynth(1- | — | 48.5 | 682 |
| (R10399) | 154_C37A_N102D)_ | | | |
| | Linker-(GGS)4- | | | |
| | GGG_Ec(J96)- | | | |
| | FimH(22-300)_Linker- | | | |
| | PGDGN_Ec(J96)-FimG(24-37) | | | |
| 13 | HslgE(1-18)_Ec(J96)-FimH(22- | 49.9 | 47.8 | 683 |
| (R10400) | 300)_Linker-PGDGN_Ec(J96)- | | | |
| | FimG(24-37)_Linker- | | | |
| | SGG_LumSynth(1- | | | |
| | 154_C37A_N102D) | | | |
| 14 | HslgE(1-18)_Ec(J96)-FimH(22- | 52.1 | 50.0 | 684 |
| (R10401) | 300)_Linker-PGDGN_Ec(J96)- | | | |
| | FimG(24-37)_Linker- | | | |
| | SGG_Ferritin(5- | | | |
| | 167_N19Q) | | | |
| 15 | MmlgK(1-20)_Ec(J96)- | 52.7 | 50.5 | 685 |
| (R10402) | FimH(22-300)_Linker- | | | |
| | PGDGN_Ec(J96)- | | | |
| | FimG(24-37)_Linker- | | | |
| | (GGS)4_Ferritin(5-166) | | | |
| 16 | HslgE(1-18)_Ec(J96)-FimH(22- | 39.3 | 37.2 | 686 |
| (R10403) | 300)_Linker-PGDGN_Ec(J96)- | | | |
| | FimG(24-37)_ | | | |
| | TMdomain-HA(510-566) | | | |

Ec: Escherichia coli; HA: Hemagglutinin; Hs: Homo sapiens; IgE: immunoglobulin E; IgK: immunoglobulin Kappa; LumSynth, LS: Lumazine synthase; Mm: Mus musculus; TMdomain, TM: transmembrane domain.

### Results:

Expression was demonstrated for most of the RNA constructs in the corresponding cell lysates (see FIG. 1A). Secretion of the tested E. coli FimH antigen designs was detected for constructs 1, 2, 3, 6, 7, 8, 9, 10 and 11 by analyzing the supernatant of transfected HeLa cells (see FIG. 1B).

### 2.2 Analysis of Immunogenicity of E. Coli FimH Antigen Designs in Mice

mRNA constructs encoding E. coli FimH antigen designs (see Table 5) were prepared according to Example 1. The mRNA was formulated with Lipid-based carrier (see Example 1.4. Preparation of a LNP formulated mRNA composition). The different mRNA vaccine candidates were applied to female BALE/c mice on day 0, 21, and 35 and were administered intramuscularly (i.m.) with 2 μg or 4 μg of RNA as shown in Table 6. A negative control group (A) received just buffer (0.9% NaCl) and one group (B) received a PHAD-adjuvanted FimHC protein complex subunit vaccine, which was obtained as described in U.S. Pat. No. 9,017,698. Serum and urine samples were taken at day 1 (18 h), 21, 35, and 49 for determination of humoral immune responses.

ELISA was performed using recombinant FimHL for coating. FimHL was obtained by cloning amino acids 22-181 of UPEC J96 FimH (GenBank: ELL41155.1) into Pet22b plasmid. Recombinant FimHL was expressed in E. coli BL21-DE3 and purified from the periplasmic space. Coated plates were incubated using respective serum or urine dilutions, and binding of specific antibodies to FimHL was detected using Peroxidase-conjugated goat anti-mouse IgG (H+L) antibody (1:5000, Jackson ImmunoResearch) followed by Amplex® UltraRed reagent (1:200, Invitrogen) as substrate. Endpoint titers of IgG antibody directed against the recombinant protein FimHL were measured by ELISA on day 21, 35, and 49

TABLE 6
Vaccination regimen (Example 2.2)

| | No. | | | | | | Immu- | | |
|---|---|---|---|---|---|---|---|---|---|
| | of | Construct # | | | | | nisation | | SEQ ID |
| Group | mice | (RNA ID) | | Dose | | Route | schedule | Samples | NO: RNA |
| A | 8 | Buffer (0.9% | | 25 | μl | i.m. | D 0 | Serum: | — |
| | | NaCl) | | | | M. tibialis | D 21 | D 1 (18 h) | |
| B | 8 | PHAD- | | 2 | μg | 2 × 25 μl | D 35 | D 21 | — |
| | | adjuvanted | | | | (protein) | | D 35 | |
| | | FimHC protein | | | | 1 × 25 μl | | D 49 | |
| | | complex | | | | (mRNA) | | Urine: | |
| | | subunit vaccine | | | | | | D 21 | |
| C | 8 | 1 | (R10388) + | 2 | μg + | | | D 35 | 671 + |
| | | 3 | (R10390) | 2 | μg | | | D 49 | 673 |
| D | 8 | 2 | (R10389) + | 2 | μg + | | | Splenocytes: | 672 + |
| | | 5 | (R10392) | 2 | μg | | | D 49 | 675 |
| E | 8 | 3 | (R10390) | 2 | μg | | | | 673 |
| F | 8 | 4 | (R10391) | 2 | μg | | | | 674 |
| G | 8 | 5 | (R10392) | 2 | μg | | | | 675 |
| H | 8 | 6 | (R10393) | 2 | μg | | | | 676 |
| I | 8 | 7 | (R10394) | 2 | μg | | | | 677 |
| J | 8 | 8 | (R10395) | 2 | μg | | | | 678 |
| K | 8 | 9 | (R10396) | 2 | μg | | | | 679 |
| L | 8 | 10 | (R10397) | 2 | μg | | | | 680 |
| M | 8 | 11 | (R10398) | 2 | μg | | | | 681 |
| N | 8 | 12 | (R10399) | 2 | μg | | | | 682 |
| O | 8 | 13 | (R10400) | 2 | μg | | | | 683 |
| P | 8 | 14 | (R10401) | 2 | μg | | | | 684 |
| Q | 8 | 15 | (R10402) | 2 | μg | | | | 685 |
| R | 8 | 16 | (R10403) | 2 | μg | | | | 686 |

Detailed antigen design of constructs/RNA IDs or SEQ ID NOs is shown in Table 3 (Example 1).

### Results:

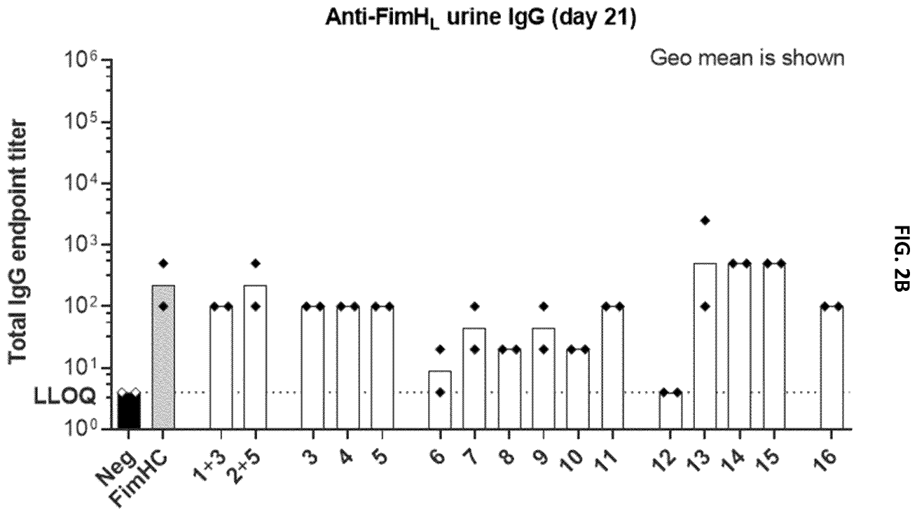

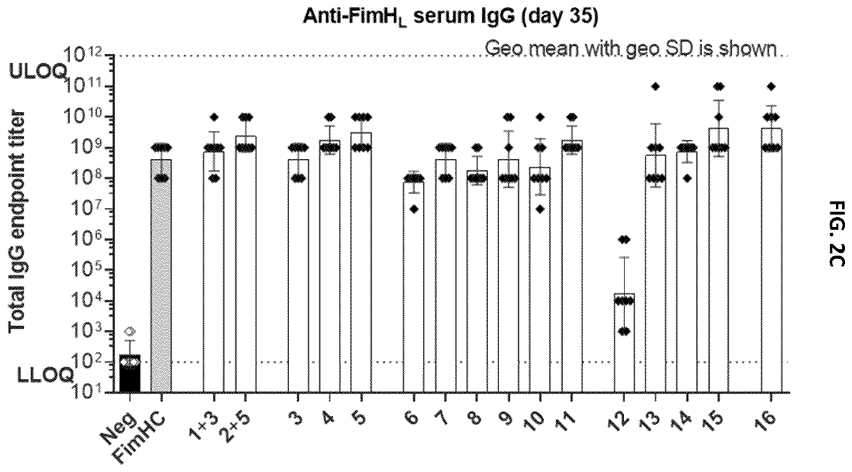

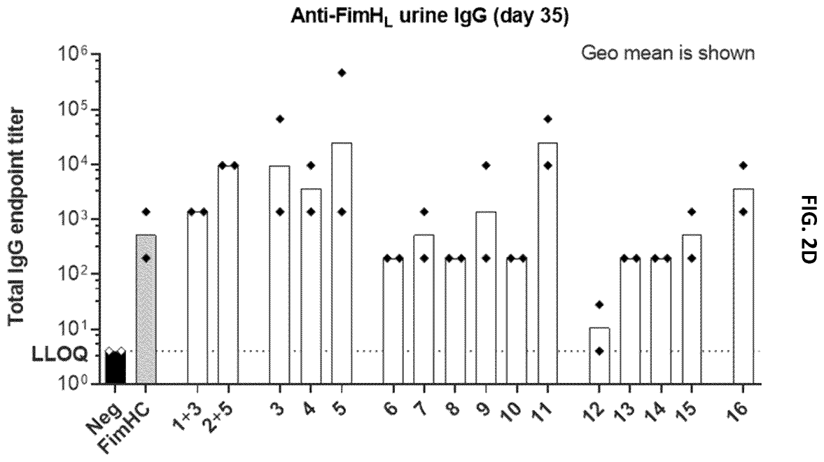

As shown in FIG. 2, the tested antigen designs induced substantial humoral immune responses in mice. FimHL-specific IgG endpoint titers (analyzed via ELISA) were detectable for most groups in serum and urine at day 21, 35 and 49. Early immune responses are very important for a fast and robust protection against UTIs. Although adaptive immune responses were already quite high after one vaccination, serum and urine antibody titers induced by vaccination with RNA vaccines or by the PHAD-adjuvanted FimHC protein complex subunit vaccine can be further increased by a second and third vaccination.

2.3 Analysis of Functional Serum Antibody Responses Against E. coli FimH Antigen Designs

The antibody response generated by immunisation with the E. coli FimH constructs as described in Example 2.2 was characterised by the inhibition of bacterial adhesion assay (BAI). BAI titres were determined using sera pools (8 mice per group) at day 21, 35 and 49 timepoints.

### Inhibition of Bacterial Adhesion Assay (BAI)

An UTI89 E. coli UPEC strain was engineered to express the mCherry fluorescent marker and was cultivated for 3 passages in static liquid culture. Bacteria were harvested, washed with PBS and resuspended at 0.012 OD₆₀₀/ml with F12K medium (Thermo Scientific) supplemented with 10% FBS without antibiotics.

Serum samples were prepared in with F12K medium or F12K supplemented with 10% FBS at a concentration twice with respect to the final working concentration (2×), and further diluted with serial dilutions. 20% D-(+)-Mannose and F12K medium supplemented with 10% FBS without antibiotics were used as positive and negative controls, respectively.

SV-HUC cells (ATCC) were cultivated in F12K medium (Thermo Scientific) supplemented with 10% FBS and antibiotics. SV-HUC cells were seeded in 96-well plates at a density of 3.5×10⁴ cells/well (final volume of 200 μl/well) and incubated at 37° C., 5% CO₂. The medium was exchanged with F12K medium supplemented with 10% FBS without antibiotics. The medium was removed and 50 μl of samples or controls were added to each well followed by 50 μl 2× bacteria inoculum or medium, as negative control. Plates were incubated for 30 minutes and serum dilution from 15% to 0.06% was added. Plates were incubated at 37° C., 5% CO₂, for 30 min and the medium was removed and the wells were washed with PBS for three times. Bacteria were fixed using 4% formaldehyde solution for 20 min, and subsequently stained with DAPI (62248, ThermoScientific) as known in the art. Microscopy analysis was performed on OPERA Phenix. Data were analysed with Harmony software. Total bacterial fluorescence area (single object ≤100 μm²) was calculated as a value of adherence. The titer for each sample was computed as the dilution corresponding to the inflection point of the dose-response curve.

### Results:

As shown in Table 7, BAI titers were detected for almost all samples at day 21. Higher inhibition titers were detected for constructs 13, 14 and 15, which encode an antigen clustering domain. A specific titer was not assigned at d21 for PHAD-adjuvanted FimHC protein complex in both the assays because below the limit of quantification, while it raised at d35 and d49. Construct 12 was below the limit of quantification for all the three timepoints. A plateau of the response was reached for most samples at day 35, as no further increase was observed at day 49. The trend observed in the BAI titers was similar to the IgG titers measured by ELISA:

TABLE 7
BAI titers of serum antibody responses against E. coli
FimH antigen designs (Example 2.3)

| Construct # | | Immunization | | | |
|---|---|---|---|---|---|
| (RNA ID) | Dose | Group | d21 | d35 | d49 |
| Negative control | 100 μl | A | — | NR | — |
| PHAD-adjuvanted | 2 μg | B | NR | 5 | 5 |
| FimHC protein | | | | | |
| complex | | | | | |
| subunit vaccine | | | | | |
| 1 (R10388) + 3 | 2 μg + | C | — | — | — |
| (R10390) | 2 μg | | | | |
| 2 (R10389) + 5 | 2 μg + | D | NR | 90 | 56 |
| (R10392) | 2 μg | | | | |
| 3 (R10390) | 2 μg | E | 6 | 220 | — |
| 4 (R10391) | 2 μg | F | 2 | 22 | — |
| 5 (R10392) | 2 μg | G | 3 | 132 | — |
| 6 (R10393) | 2 μg | H | 3 | 157 | — |
| 7 (R10394) | 2 μg | I | 4 | 127 | — |
| 8 (R10395) | 2 μg | J | 2 | 299 | — |
| 9 (R10396) | 2 μg | K | 3 | 373 | 354 |
| 10 (R10397) | 2 μg | L | — | — | — |
| 11 (R10398) | 2 μg | M | 3 | 106 | — |
| 12 (R10399) | 2 μg | N | NR | NR | — |
| 13 (R10400) | 2 μg | O | 10 | 230 | 292 |
| 14 (R10401) | 2 μg | P | 18 | 251 | 834 |
| 15 (R10402) | 2 μg | Q | 11 | 133 | 207 |
| 16 (R10403) | 2 μg | R | 3 | 100 | — |

NR (not responder) indicates tested sample lower than the limit of quantification, “—” indicates that the assay was not performed.

2.4 Analysis of T-Cell Responses of E. coli FimH Antigen Designs Using Intracellular Cytokine Staining (ICS) and FACS

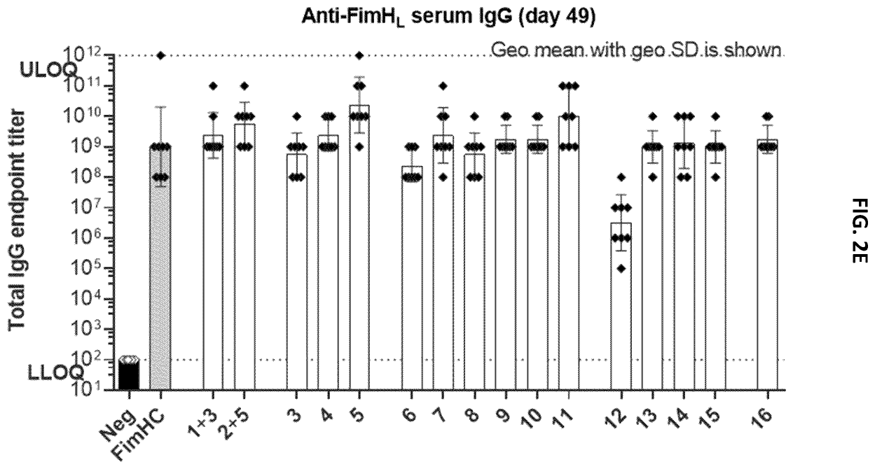

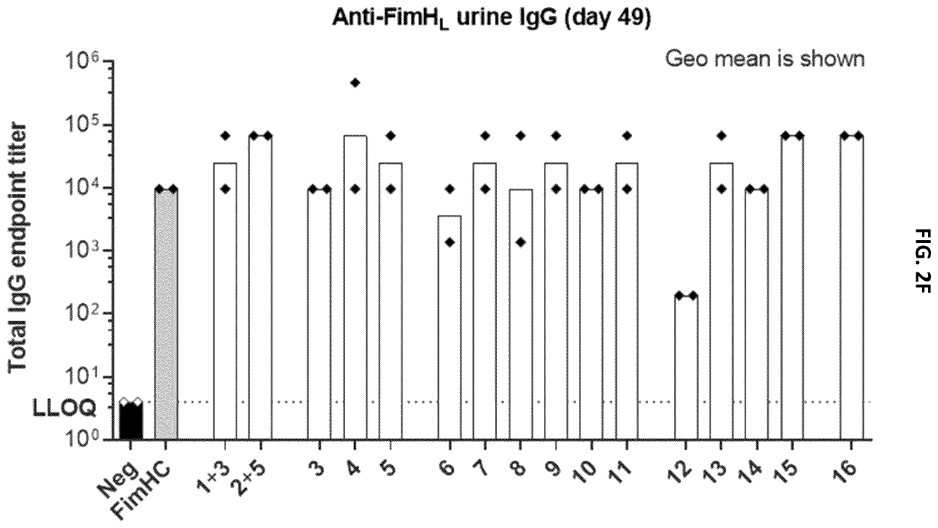

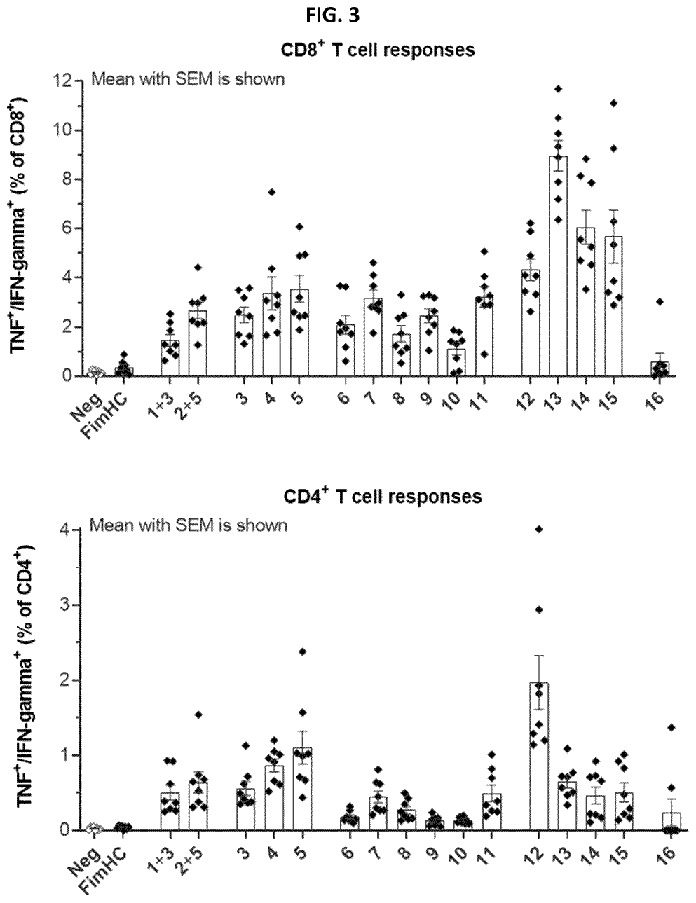

Splenocytes from vaccinated and control mice were isolated on day 49 according to a standard protocol known in the art. Briefly, isolated spleens were grinded through a cell strainer and washed in PBS/1% FBS followed by red blood cell lysis. After an extensive washing step with PBS/1% FBS, splenocytes were seeded into 96-well plates (2×10⁶ cells per well). Cells were stimulated with a mixture of FimH protein specific peptides (1 μg/ml each) in the presence of 2.5 μg/ml of an anti-CD28 antibody, an anti-CD107a PE-Cy7 antibody (1:100) and a protein transport inhibitor (BD Biosciences) for 6 hours at 37° C. After stimulation, cells were washed and stained for intracellular cytokines using the Cytofix/Cytoperm solution (BD Biosciences) according to the manufacturer's instructions. The following antibodies were used for staining: anti-Thy1.2 FITC (1:200, Biolegend), anti-CD8 APC-H7 (1:200, BD Biosciences), anti-CD4 BD Horizon™ V450 (1:200, BD Biosciences), anti-TNFa PE (1:100, eBioscience), anti-IFNg APC (1:100, BD Biosciences) and incubated with Fc-block diluted 1:100. Aqua Dye was used to distinguish live/dead cells (Invitrogen). Cells were acquired using a ZE5 flow cytometer (Bio-Rad). Flow cytometry data was analyzed using FlowJo software package (Tree Star, Inc.). Table 6 (Vaccination regimen of Example 2.2) contains mRNA constructs that were used in the experiment and the result of the experiment is shown in FIG. 3.

### Results:

As shown in FIG. 3, most of the LNP formulated ExPEC FimH vaccines led to a significant induction of cellular immune responses with detectable frequencies of FimH-specific CD4+ T helper cells and CD8+ cytotoxic T cells producing IFN-gamma and TNF two weeks after the third vaccination on day 49, while there were no measurable T cell responses for the PHAD-adjuvanted FimHC protein complex.

### Example 3: Analysis of E. coli FimH Designs in Rats

3.1 In Vivo Analysis of Immunogenicity of E. coli FimH Designs

mRNA constructs encoding E. coli FimH designs (see Table 3) were prepared according to Example 1. The mRNA was formulated with Lipid-based carrier (see Example 1.4. Preparation of a LNP formulated mRNA composition). The different mRNA vaccine candidates were applied to female Wistar rats on day 0, 21, and 35 and were administered intramuscularly (i.m.) with 1 μg, 4 μg or 12 μg of RNA as shown in Table 8. A negative control group (A) received just buffer (0.9% NaCl) and three groups (B, C, D) received an AS01-adjuvanted FimHdG protein subunit vaccine (0.71 μg, 2.83 μg or 8.49 μg), which was obtained by cloning in the pET24b (+) vector a sequence corresponding to SEQ ID NO: 504 (without a signal peptide). Recombinant FimHdG was expressed in E. coli and purified from inclusion bodies using techniques known in the art. Serum and urine samples were taken at day 1 (18 h), 21, 35, and 49 for determination of humoral immune responses.

ELISA was performed using recombinant protein FimHL for coating as described in Example 2.2. Coated plates were incubated using respective serum or urine dilutions, and binding of specific antibodies to the respective recombinant protein FimHL was detected using goat anti-rat IgG (whole molecule)-Peroxidase antibody (1:5000, Sigma-Aldrich) followed by Amplex® UltraRed reagent (1:200, Invitrogen) as substrate. Endpoint titers of IgG antibody directed against the recombinant protein FimHL were measured by ELISA on day 21, 35, and 49.

TABLE 8
Vaccination regimen (Example 3.1)

| | No. | | | | | Immu- | | |
|---|---|---|---|---|---|---|---|---|
| | of | Construct # | | | | nisation | | SEQ ID |
| Group | rats | (RNA ID) | Dose | | Route | schedule | Samples | NO: RNA |
| A | 7 | Buffer | 100 | μl | i.m. | D 0 | Serum: | — |
| | | (0.9% NaCl) | | | M. | D 21 | D 1 (18 h) | |
| B | 7 | | 0.7 | μg | gastrocnemius | D 35 | D 21 | — |
| C | 7 | | 2.8 | μg | 1 × 100 μl | | D 35 | |
| | | | | | (protein) | | D 49 | |
| D | 7 | AS01-adjuvanted | 8.5 | μg | 1 × 100 μl | | Urine: | |
| | | FimHdG | | | (mRNA) | | D 21 | |
| | | subunit vaccine | | | | | D 35 | |
| E | 7 | 9 | 1 | μg | | | D 49 | 679 |
| F | 7 | (R10396) | 4 | μg | | | Splenocytes: | |
| G | 7 | | 12 | μg | | | D 49 | |
| H | 7 | 13 | 1 | μg | | | | 683 |
| I | 7 | (R10400) | 4 | μg | | | | |
| J | 7 | | 12 | μg | | | | |
| K | 7 | 14 | 1 | μg | | | | 684 |
| L | 7 | (R10401) | 4 | μg | | | | |
| M | 7 | | 12 | μg | | | | |

Detailed antigen design of constructs/RNA IDs or SEQ ID NOs is shown in Table 3 (Example 1).

### Results:

As shown in FIG. 4, the different antigen designs induced substantial humoral immune responses in a dose dependent manner in Wistar rats. FimHL-specific IgG endpoint titers (analyzed via ELISA) were detectable in serum and urine samples for most groups on at least one day after immunization (day 21, 35 and/or 49). Early immune responses are very important for a fast and robust protection against UPEC infections. Although adaptive immune responses were already quite high for some groups after one vaccination, serum and urine antibody titers induced by vaccination with RNA vaccines or by the protein subunit vaccine can be further increased by a second and third vaccination. The highest titers were detected for groups immunized with nanoparticle-forming antigen designs of RNA constructs, already after one dose, with construct 14 inducing higher titers than construct 13. Dose responses were most pronounced between groups immunized with 1 μg or 4 μg of RNA vaccines.

### 3.2 Analysis of Functional Serum Antibody Responses Against E. Coli FimH Antigen Designs

The antibody response generated by immunisation with the E. coli FimH constructs as described in Example 3.1 was characterised by the inhibition of bacterial adhesion assay (BAI). The BAI assay was run as described in Example 2.3.

### Results:

As shown in Table 9, at day 21, BAI titers were observed only for samples 13 and 14, which encode an antigen clustering domain, at 4 μg and 12 μg dosage.

BAI titers increased at day 35 and 49. Furthermore, a dose response accounting for increasing doses (1, 4 and 12 μg) could be detected. Moreover, constructs 13 and 14, which encode an antigen clustering domain, showed higher BAI titers at all the three dosages in comparison to the single subunit mRNA construct 9. Furthermore, the BAI titers of the FimHdG-AS01 protein vaccine were lower than those of the RNA vaccines. However, while the dose of RNA or proteins administered to rats appear similar in μg quantities no direct comparison between mRNA and protein dosages can be made. This could be due to a range of protein doses that was possibly suboptimal and not reaching maximal responses for responses in Rat Spp. in this study.

TABLE 9
BAI titers of serum antibody responses against E. coli FimH
antigen designs (Example 3.2)

| Construct # | | Immunization | | | |
|---|---|---|---|---|---|
| (RNA ID) | Dose | Group | d21 | d35 | d49 |
| Negative control | 100 μl | A | NR | NR | NR |
| AS01-adjuvanted | 0.7 μg | B | NR | NR | NR |
| FimHdG protein | 2.8 μg | C | NR | NR | 17 |
| subunit vaccine | 8.5 μg | D | NR | NR | 24 |
| 9 (R10396) | 1 μg | E | NR | 21 | 29 |
| | 4 μg | F | 3 | 80 | 142 |
| | 12 μg | G | 3 | 64 | 73 |
| 13 (R10400) | 1 μg | H | 3 | 99 | 377 |
| | 4 μg | I | 12 | 418 | 686 |
| | 12 μg | J | 15 | 207 | 835 |
| 14 (R10401) | 1 μg | K | 3 | 99 | 189 |
| | 4 μg | L | 21 | 440 | 855 |
| | 12 μg | M | 10 | 526 | 586 |

NR indicates sample not responder, “—” indicates that the assay was not analysed to quantify a titer. Due to the low functionality observed after day 21, a titer equal to 3 is assigned to the samples with titer lower than the limit of quantification which did not show a flat inhibition curve, as the one observed for the negative control (NR).

### Example 4: Analysis of E. coli FimH antigen designs with unmodified versus chemically modified mRNA

### 4.1 In Vitro Analysis of Expression and Secretion of E. Coli FimH Antigen Designs Using Western Blot

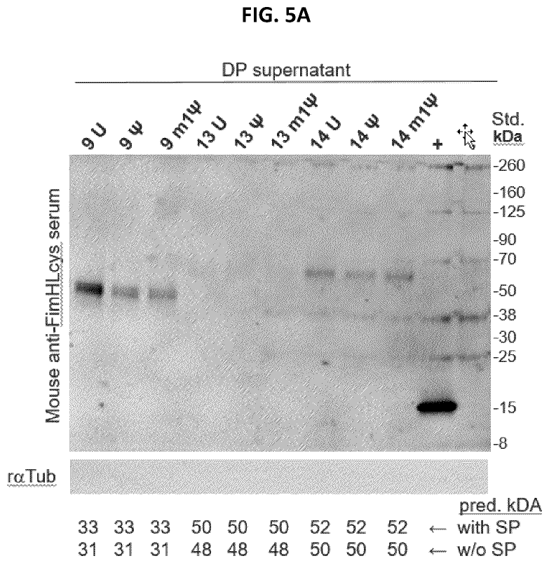

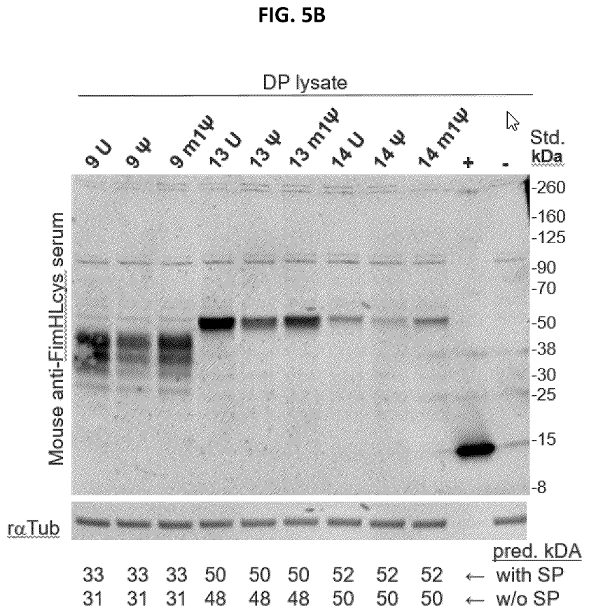

To determine the in vitro protein expression of some of the mRNA constructs, HEK 293T cells were transfected with 0.5 μg LNP formulated mRNA encoding different antigen design using 6-well plates. 48 h after transfection, cell lysates and cell culture supernatants were subjected to SOS-PAGE and Western blot analysis using mouse anti-FimHLcys serum (1:1000, as described in Example 2.1) or rabbit anti-alpha-tubulin antibody (1:1000; Cell Signaling) as primary antibodies as well as goat anti-rabbit IgG IRDye® 680RD antibody (1:10000; Li-Cor) or goat anti-mouse IgG IRDye® 8000W antibody (1:10000; Li-Cor) as secondary antibodies. Detection and quantification was performed using a Li-Cor detection system (Odyssey CLx image system) in combination with Image Studio Lite software. Table 10 contains mRNA constructs that were used in the experiment and the result of the experiment is shown in FIG. 5.

TABLE 10
mRNA constructs used for Western blot analysis (Example 4.1)

| | | Pre- | Pre- | |
|---|---|---|---|---|
| | | dicted | dicted | |
| | | size in | size in | |
| Con- | | kDa | kDa | |
| struct | | with | w/o | SEQ ID |
| # (RNA | Antigen design; | signal | signal | NO: |
| ID) | used nucleotides | peptide | peptide | RNA |
| Negative | Medium | — | — | — |
| control | | | | |
| Subunit | Recombinant FimHL protein | 16.9 | | — |
| vaccine | (100 ng loaded on each gel) | (FimHL) | | |
| 9 | MmlgK(1-20)_Ec(J96)- | 33.1 | 30.9 | 679 |
| (R10396) | FimH(22-300)_Linker- | | | |
| | PGDGN_Ec(J96)- | | | |
| | FimG(24-37); | | | |
| | unmodified: uridine (U) | | | |
| 9 | MmlgK(1-20)_Ec(J96)- | 33.1 | 30.9 | 829, |
| (R10949) | FimH(22-300)_Linker- | | | 1274 |
| | PGDGN_Ec(J96)- | | | |
| | FimG(24-37); | | | |
| | modified: pseudouridine (ψ) | | | |
| 9 | MmlgK(1-20)_Ec(J96)- | 33.1 | 30.9 | 829, |
| (R10950) | FimH(22-300)_Linker- | | | 1271 |
| | PGDGN_Ec(J96)- | | | |
| | FimG(24-37); | | | |
| | modified: N1- | | | |
| | methylpseudouridine (m1ψ) | | | |
| 13 | HslgE(1-18)_Ec(J96)- | 49.9 | 47.8 | 683 |
| (R10400) | FimH(22-300)_Linker- | | | |
| | PGDGN_Ec(J96)- | | | |
| | FimG(24-37)_Linker- | | | |
| | SGG_LumSynth(1- | | | |
| | 154_C37A_N102D); | | | |
| | unmodified: uridine (U) | | | |
| 13 | HslgE(1-18)_Ec(J96)- | 49.9 | 47.8 | 833, |
| (R10951) | FimH(22-300)_Linker- | | | 1275 |
| | PGDGN_Ec(J96)- | | | |
| | FimG(24-37)_Linker- | | | |
| | SGG_LumSynth(1- | | | |
| | 154_C37A_N102D); | | | |
| | modified: pseudouridine (ψ) | | | |
| 13 | HslgE(1-18)_Ec(J96)- | 49.9 | 47.8 | 833, |
| (R10952) | FimH(22-300)_Linker- | | | 1272 |
| | PGDGN_Ec(J96)- | | | |
| | FimG(24-37)_Linker- | | | |
| | SGG_LumSynth(1- | | | |
| | 154_C37A_N102D); | | | |
| | modified: N1- | | | |
| | methylpseudouridine (m1ψ) | | | |
| 14 | HslgE(1-18)_Ec(J96)- | 52.1 | 50.0 | 684 |
| (R10401) | FimH(22-300)_Linker- | | | |
| | PGDGN_Ec(J96)- | | | |
| | FimG(24-37)_Linker- | | | |
| | SGG_Ferritin(5-167_N19Q); | | | |
| | unmodified: uridine (U) | | | |
| 14 | HslgE(1-18)_Ec(J96)- | 52.1 | 50.0 | 834, |
| (R10953) | FimH(22-300)_Linker- | | | 1276 |
| | PGDGN_Ec(J96)- | | | |
| | FimG(24-37)_Linker- | | | |
| | SGG_Ferritin(5-167_N19Q); | | | |
| | modified: pseudouridine (ψ) | | | |
| 14 | HslgE(1-18)_Ec(J96)- | 52.1 | 50.0 | 834, |
| (R10954) | FimH(22-300)_Linker- | | | 1273 |
| | PGDGN_Ec(J96)- | | | |
| | FimG(24-37)_Linker- | | | |
| | SGG_Ferritin(5-167_N19Q); | | | |
| | modified: N1- | | | |
| | methylpseudouridine (m1ψ) | | | |

### Results:

Expression of all nine unmodified and modified RNA constructs was demonstrated in the corresponding cell lysates (see FIG. 51B). Secretion of the tested E. coli FimH antigen designs (unmodified and modified) was detected for construct 9 and 14 by analyzing the supernatant of transfected HEK 293T cells (see FIG. 5A).

### 4.2 Analysis of Immunogenicity of E. Coli FimH Designs in Rats

mRNA constructs encoding different E. coli FimH antigen designs (see Table 3) were prepared according to Example 1. The mRNA was formulated with Lipid-based carrier (see Example 1.4. Preparation of a LNP formulated mRNA composition). The different mRNA vaccine candidates were applied to female Wistar rats on day 0, 21, and 35 and were administered intramuscularly (i.m.) with 1 μg or 12 μg of unmodified RNA or of pseudouridine (ψ) or N1-methylpseudouridine (mL) modified RNA as shown in Table 11. A negative control group (A) received just buffer (0.9% NaCl) and two groups (B, C) received an AS01-adjuvanted FimHdG protein subunit vaccine. Serum and urine samples were taken at day 1 (18 h), 21, 35, and 49 for determination of humoral immune responses.

ELISA was performed using recombinant protein FimHL for coating described in Example 2.2. Coated plates were incubated using respective serum or urine dilutions, and binding of specific antibodies to the respective recombinant protein FimHL was detected using goat anti-rat IgG (whole molecule)-Peroxidase antibody (1:5000, Sigma-Aldrich) followed by Amplex® UltraRed reagent (1:200, Invitrogen) as substrate. Endpoint titers of IgG antibody directed against the recombinant protein FimHL were measured by ELISA on day 21, 35, and 49.

TABLE 11
Vaccination regimen (Example 4.2)

| | No. | Construct # | | | | Immu- | | |
|---|---|---|---|---|---|---|---|---|
| | of | (RNA ID); | | | | nisation | | SEQ ID |
| Group | rats | used nucleotides | Dose | | Route | schedule | Samples | NO: RNA |
| A | 8 | Buffer | 100 | μl | i.m. | D 0 | Serum: | — |
| | | (0.9% NaCl) | | | M. | D 21 | D 1 (18 h) | |
| B | 8 | AS01-adjuvanted | 1 | μg | gastrocnemius | D 35 | D 21 | |
| C | 8 | FimHdG protein | 12 | μg | 1 × 100 μl | | D 35 | |
| | | subunit vaccine | | | (protein) | | D 49 | |
| D | 8 | 14 | 1 | μg | 1 × 100 μl | | Urine: | 684 |
| E | 8 | (R10401); | 12 | μg | (mRNA) | | D 21 | |
| | | unmodified: uridine | | | | | D 35 | |
| | | (U) | | | | | D 49 | |
| F | 8 | 14 | 1 | μg | | | Splenocytes: | 834, |
| G | 8 | (R10953); | 12 | μg | | | D 49 | 1276 |
| | | modified: | | | | | | |
| | | pseudouridine (ψ) | | | | | | |
| H | 8 | 14 | 1 | μg | | | | 834, |
| I | 8 | (R10954); | 12 | μg | | | | 1273 |
| | | modified: N1- | | | | | | |
| | | methylpseudouridine | | | | | | |
| | | (m1ψ) | | | | | | |

Detailed antigen design of constructs/RNA IDs or SEQ ID NOs is shown in Table 3 (Example 1).

### Results:

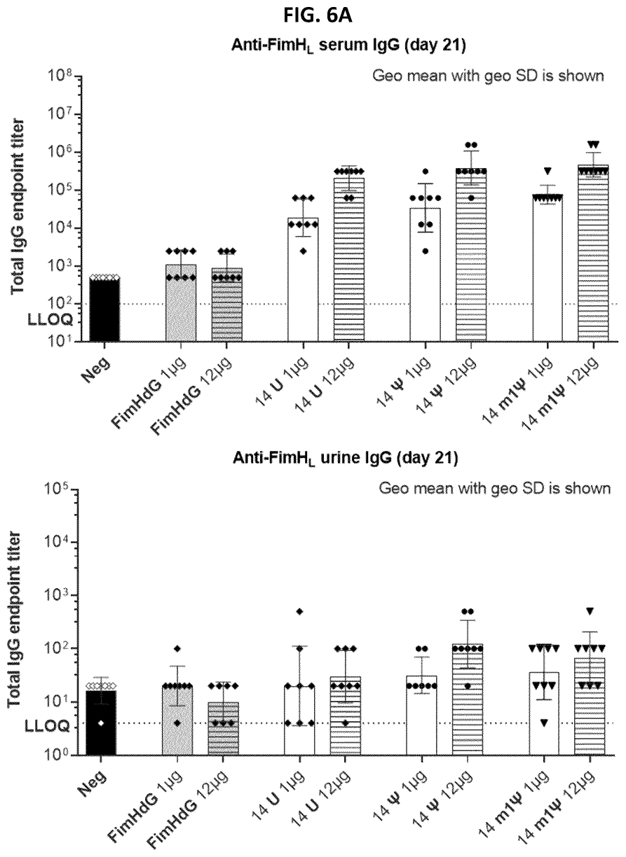

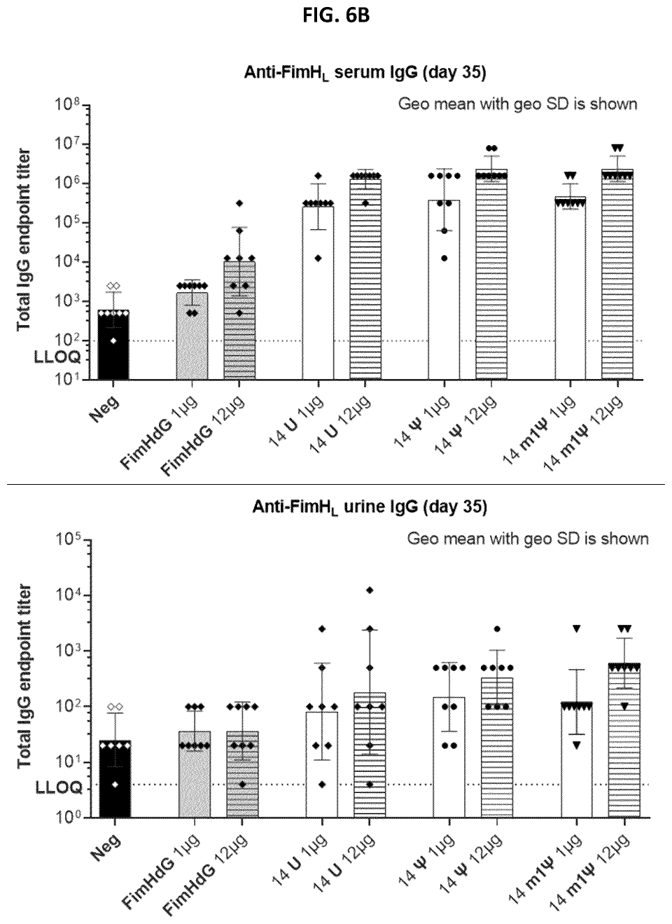

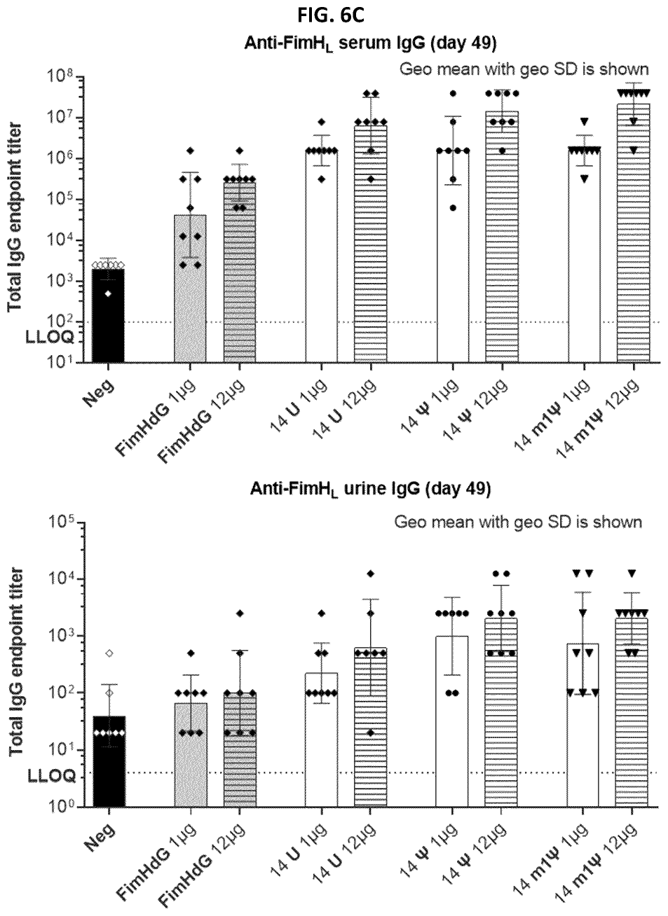

As shown in FIG. 6, unmodified and pseudouridine (ψ) or N1-methylpseudouridine (m1ψ) modified LNP formulated E. coli FimH RNA vaccines of the tested construct 14 induced substantial humoral immune responses in a dose dependent manner in Wistar rats. FimHL-specific IgG endpoint titers (analyzed via ELISA) were detectable in serum and urine samples for most groups on at least one day after immunization (day 21, 35 and/or 49). Early immune responses are very important for a fast and robust protection against UPEC infections. Although adaptive immune responses were already quite high for some groups after one vaccination, serum and urine antibody titers induced by vaccination with RNA vaccines or by the protein subunit vaccine can be further increased by a second and third vaccination. Groups vaccinated with ψ- and especially m1ψ-containing RNA vaccines tended to have slightly higher urine and serum titers compared with groups vaccinated with the unmodified RNA vaccine.

### 4.3 Analysis of Functional Serum Antibody Responses Against E. Coli FimH Antigen Designs

The functional antibody response generated by immunisation with the E. coli FimH constructs as described in Example 4.2 was measured by the inhibition of bacterial adhesion assay (BAI). The BAI assay was run as described in Example 2.3.

### Results:

As shown in Table 12, at day 21, BAI titers were observed for unmodified and pseudouridine (ψ) or N1-methylpseudouridine (m1ψ) modified LNP formulated E. coli FimH RNA vaccines of the tested construct 14 at the higher dose. BAI titers increased at day 35 and 49. Furthermore, the BAI titers of the FimHdG-AS01 protein subunit vaccine were lower than those of the RNA vaccines.

TABLE 12
BAI titers of serum antibody responses against E. coli
FimH antigen designs (Example 4.3)

| | | Immunization | | | |
|---|---|---|---|---|---|
| Construct # (RNA ID) | Dose | Group | d21 | d35 | d49 |
| Buffer | 100 μl | A | NR | NR | NR |
| (0.9% NaCl) | | | | | |
| AS01-adjuvanted FimHdG protein | 1 μg | B | NR | NR | 7 |
| subunit vaccine | 12 μg | C | NR | NR | 27 |
| 14 (R10401); | 1 μg | D | NR | 37 | 74 |
| unmodified: uridine (U) | 12 μg | E | 8 | 83 | 136 |
| 14 (R10953); | 1 μg | F | NR | 71 | 229 |
| modified: pseudouridine (ψ) | 12 μg | G | 19 | 160 | 293 |
| 14 (R10954); | 1 μg | H | NR | 88 | 380 |
| modified: N1-methylpseudouridine | 12 μg | I | 24 | 539 | 405 |
| (m1ψ) | | | | | |

NR (not responder) indicates tested sample lower than the corresponding limit of quantification.

TABLE 13
additional sequences shorter than 10 specifically defined nucleotides or 4
specifically defined amino acids

| SEQ ID | | Sequence | Sequence | |
|---|---|---|---|---|
| NO: | Description | Length | Type | Sequence |
| 121 | mRNA 5′-end | 6 | RNA | GGGAGA |
| 122 | mRNA 5′-end | 6 | RNA | AGGAGA |
| 123 | mRNA 5′-end | 6 | RNA | GGGAAA |
| 124 | mRNA 5′-end | 6 | RNA | AGAAUA |
| 125 | mRNA 5′-end | 6 | RNA | AGAUUA |
| 126 | mRNA 5′-end | 6 | RNA | GAUGGG |
| 127 | mRNA 5′-end | 5 | RNA | GGGCG |
| 130 | Kozak sequence | 9 | DNA | GCCGCCACC |
| 131 | Kozak sequence | 9 | RNA | GCCGCCACC |
| 132 | Kozak sequence | 6 | DNA | GCCACC |
| 133 | Kozak sequence | 6 | RNA | GCCACC |
| 134 | Kozak sequence | 3 | DNA | ACC |
| 135 | Kozak sequence | 3 | RNA | ACC |
| 355 | Artificial_Linker-SGG protein | 3 | protein | SGG |
| 362 | Artificial_Linker-SGG CDS opt1 | 9 | RNA | AGCGGCGGG |
| 369 | Artificial_Linker-SGG CDS opt1 | 9 | RNA | AGCGGCGGG |
| 376 | Artificial_Linker-SGG CDS opt3 | 9 | RNA | AGCGGCGGG |
| 383 | Artificial_Linker-SGG CDS opt4 | 9 | RNA | AGCGGCGGC |
| 390 | Artificial_Linker-SGG CDS opt5 | 9 | RNA | AGCGGCGGG |

1. A coding RNA comprising at least one untranslated region (UTR) and at least one coding sequence encoding an antigenic polypeptide which is selected or derived from Escherichia coli type 1 fimbriae D-mannose specific adhesin (FimH).

2. The coding RNA according to claim 1, wherein the E. coli FimH comprises an amino acid sequence which is identical or at least 70%, 80%, 85%, 86%, 87%, 88%, 89%, 90%, 91%, 92%, 93%, 94%, 95%, 96%, 97%, 98%, or 99% identical to any one of SEQ ID NOs: 177-186, 247-256 or is an immunogenic fragment or immunogenic variant thereof.

3. The coding RNA according to claims 1 or 2, wherein the coding sequence additionally encodes one or more further peptide or protein elements selected from: a donor strand peptide, a signal peptide, an antigen clustering domain, or a transmembrane domain.

4. The coding RNA according to claim 3, wherein the one or more further peptide or protein element(s) is a donor strand peptide, optionally wherein the coding sequence encodes the following elements in N-terminal to C-terminal direction: the antigenic polypeptide which is selected or is derived from E. coli FimH; and the donor strand peptide.

5. The coding RNA according to claim 4, wherein the donor strand peptide comprises or consists of the amino acid sequence of SEQ ID NO: 338 or a variant thereof, optionally wherein the variant of SEQ ID NO: 338 has from 1 to 5, such as 1, 2, 3 or 4 single amino acid mutations compared to SEQ ID NO: 338.

6. The coding RNA according to any one of claims 4 or 5, wherein the coding sequence additionally encodes a peptide linker, optionally wherein the coding sequence encodes the following elements in N-terminal to C-terminal direction: the antigenic polypeptide which is selected or derived from E. coli FimH; the peptide linker element; and the donor strand peptide.

7. The coding RNA according to claim 6, wherein the peptide linker comprises or consists of SEQ ID NO: 352.

8. The coding RNA according to any one of claims 3 to 7, wherein the coding sequence additionally encodes an antigen clustering domain, optionally wherein the antigen clustering domain is selected or derived from ferritin or lumazine synthase.

9. The coding RNA according to claim 8, wherein the amino acid sequence of the antigen clustering domain is identical or at least 70%, 80%, 85%, 86%, 87%, 88%, 89%, 90%, 91%, 92%, 93%, 94%, 95%, 96%, 97%, 98%, or 99% identical to any one of amino acid sequences SEQ ID NOs: 457-459, 443, 444, or fragment or variant thereof.

10. The coding RNA according to any one of claims 3 to 9, wherein the coding sequence additionally encodes a signal peptide, optionally wherein the signal peptide is selected or derived from IgE or IgK, wherein the amino acid sequences of the signal peptides is identical or at least 70%, 80%, 85%, 86%, 87%, 88%, 89%, 90%, 91%, 92%, 93%, 94%, 95%, 96%, 97%, 98%, or 99% identical to any one of amino acid sequences SEQ ID NOs: 394, 395, or fragment or variant thereof.

11. The coding RNA according to any one of claims 3 to 10, wherein the coding sequence encodes the following elements optionally in N-terminal to C-terminal direction:
a) signal peptide, antigenic polypeptide;
b) signal peptide, antigenic polypeptide, peptide linker, donor strand peptide;
c) antigen clustering domain, peptide linker, antigenic polypeptide, peptide linker, donor strand peptide;
d) signal peptide, antigen clustering domain, peptide linker, antigenic polypeptide, peptide linker, donor strand peptide;
e) signal peptide, antigenic polypeptide, peptide linker, donor strand peptide, peptide linker, antigen clustering domain; or
f) signal peptide, antigenic polypeptide, peptide linker, donor strand peptide, peptide linker, transmembrane domain.

12. The coding RNA according to any one of the preceding claims, wherein the coding sequence encodes an amino acid sequence which is identical or at least 70%, 80%, 85%, 86%, 87%, 88%, 89%, 90%, 91%, 92%, 93%, 94%, 95%, 96%, 97%, 98%, or 99% identical to any one of SEQ ID NOs: 177-186, 247-256, 498-520, 1277, or an immunogenic fragment or immunogenic variant thereof.

13. The coding RNA according to any one of the preceding claims, wherein the coding sequence comprises a nucleic acid sequences which is identical or at least 70%, 80%, 85%, 86%, 87%, 88%, 89%, 90%, 91%, 92%, 93%, 94%, 95%, 96%, 97%, 98%, or 99% identical to the sequences according to any one of SEQ ID NOs: 187-246, 257-316, 523-545, 548-570, 573-595, 598-620, 623-645, 648-670, or a fragment or a variant thereof.

14. The coding RNA according to any one of the preceding claims, wherein the coding sequence comprises at least one modified nucleotide selected from pseudouridine (ψ) and N1-methylpseudouridine (m1ψ), optionally wherein essentially all uracil nucleotides are replaced by pseudouridine (ψ) nucleotides and/or N1-methylpseudouridine (m1ψ) nucleotides.

15. The coding RNA according to any one of the preceding claims, wherein the coding sequence is a codon modified coding sequence, wherein the amino acid sequence encoded by the at least one codon modified coding sequence is optionally not being modified compared to the amino acid sequence encoded by the corresponding wild type coding sequence, optionally wherein the at least one codon modified coding sequence is selected from C maximized coding sequence, codon adaptation index (CAI) maximized coding sequence, human codon usage adapted coding sequence, G/C content modified coding sequence, and G/C optimized coding sequence, or any combination thereof.

16. The coding RNA according to any one of the preceding claims, wherein the coding RNA is an mRNA, optionally comprising or consisting of a nucleic acid sequence which is identical or at least 70%, 80%, 85%, 86%, 87%, 88%, 89%, 90%, 91%, 92%, 93%, 94%, 95%, 96%, 97%, 98%, or 99% identical to a nucleic acid sequence according to any one of SEQ ID NOs: 673-695, 698-720, 723-745, 748-770, 773-795, 798-820, 823-845, 848-870, 873-895, 898-920, 923-945, 948-970, 973-995, 998-1020, 1023-1045, 1048-1070, 1073-1095, 1098-1120, 1123-1145, 1148-1170, 1173-1195, 1198-1220, 1223-1245, 1248-1276 or a fragment or variant thereof.

17. A pharmaceutical composition comprising the coding RNA according to any one of claims 1 to 16.

18. The pharmaceutical composition according to claim 17, further comprising lipid-base carriers, wherein the lipid-base carriers are lipid nanoparticles (LNP).

19. A vaccine comprising the coding RNA according to any one of claims 1 to 16, or the pharmaceutical composition according to any one of claims 17 or 18.

20. A Kit or kit of parts, comprising the coding RNA according to any one of claims 1 to 16, the pharmaceutical composition according to any one of claims 17 or 18, and/or the vaccine according to claim 19, optionally comprising a liquid vehicle for solubilising, and, optionally, technical instructions providing information on administration and dosage of the components.

21. The coding RNA according to any one of claims 1 to 16, the pharmaceutical composition according to any one of claims 17 or 18, the vaccine according to claim 19, or the kit or kit of parts according to claim 19, for use as a medicament.

22. The coding RNA according to any one of claims 1 to 17, the pharmaceutical composition according to any one of claims 18 or 19, the vaccine according to claim 20, or the kit or kit of parts according to claim 21, for use in treating or preventing one or more symptoms associated with urinary tract infections (UTI) in a subject in need thereof.

23. The coding RNA according to any one of claims 1 to 16, the pharmaceutical composition according to any one of claims 17 or 18, the vaccine according to claim 19, or the kit or kit of parts according to claim 20, for use in treating or preventing a disease caused by E. coli.

24. A method of treating or preventing a disorder, wherein the method comprises administering to a subject in need thereof an effective amount of the coding RNA according to any one of claims 1 to 16, the pharmaceutical composition according to any one of claims 17 or 18, the vaccine according to claim 21, or the kit or kit of parts according to claim 20.
